(19) United States
(12) Patent Application Publication
(10) Pub. No.: US 2021/0348171 A1
(43) Pub. Date: Nov. 11, 2021

(54) PHARMACOPROTEOMICS PLATFORM IDENTIFYING KINOME FEATURES REGULATING DRUG RESPONSE IN CANCER

(71) Applicants: University of Washington, Seattle, WA (US); Fred Hutchinson Cancer Research Center, Seattle, WA (US)

(72) Inventors: Shao-En Ong, Seattle, WA (US); Martin Golkowski, Seattle, WA (US); Ho-Tak Lau, Seattle, WA (US); Taranjit S. Gujral, Seattle, WA (US)

(73) Assignees: University of Washington, Seattle, WA (US); Fred Hutchinson Cancer Research Center, Seattle, WA (US)

(21) Appl. No.: 17/313,512

(22) Filed: May 6, 2021

Related U.S. Application Data
(60) Provisional application No. 63/021,501, filed on May 7, 2020.

(51) Int. Cl. C12N 15/113; A61K 45/06; A61P 35/00; A61K 31/7088; C07K 16/22; A61K 39/395; C07K 16/28; A61K 31/47; A61K 31/455

(52) U.S. Cl. CPC C12N 15/1137; A61K 45/06; A61P 35/00; A61K 31/7088; C07K 16/22; A61K 39/3955; C07K 2317/21; C07K 16/2827; A61K 31/47; A61K 31/455; C12N 2310/14; C12N 2320/31; C07K 2317/24; C07K 16/2863

(57) ABSTRACT

The disclosure provides methods and compositions for increasing sensitivity, or decreasing resistance, of cancer cells to chemotherapeutic agents such as kinase inhibitor agents. In some embodiments, the cancer cells are hepatocellular carcinoma (HCC) cells. The methods and compositions can be integrated into methods of treatment of a subject with cancer, which can further comprise administering a chemotherapeutic agent such as kinase inhibitor agents. In another aspect, the disclosure provides a method for profiling the kinome of a cell or group of similar cells that incorporates kinase capture reagents and mass spectrometry analysis.

### CROSS-REFERENCE TO RELATED APPLICATION

This application claims the benefit of U.S. Provisional Application No. 63/021,501, filed May 7, 2020, the disclosure of which is hereby expressly incorporated herein by reference in its entirety.

### STATEMENT OF GOVERNMENT LICENSE RIGHTS

This invention was made with Government support under Grant Nos. R01 AR065459, R01 GM129090, R21 CA177402 and R21 EB018384, awarded by the National Institutes of Health. The Government has certain rights in the invention.

### BACKGROUND

Hepatocellular carcinoma (HCC) is the fourth most common cause of cancer-related death worldwide and has many etiologies, including viral hepatitis, alcoholic cirrhosis, and nonalcoholic steatohepatitis (NASH). Among solid cancers, HCC has one of the fewest druggable genetic alterations, limiting treatment options for advanced HCC. Five of the seven FDA-approved drugs for advanced HCC target protein kinases, including the small molecule drugs sorafenib, regorafenib, lenvatinib and cabozantinib, as well as the antibody Ramucirumab, highlighting the importance of kinase-dependent signaling networks in HCC progression. However, predictive biomarkers that could guide clinical use of these kinase inhibitors (KI) are lacking, likely contributing to the poor response rates of 10-15%.

Even in HCCs that initially respond to treatment, drug resistance invariably develops. This has been particularly well-documented for sorafenib and suggests that HCCs activate compensatory signaling pathways to drive rebound growth. In carcinomas, many of these compensatory pathways are linked to the interconversion between an epithelial-like to a mesenchymal-like cancer cell phenotype, i.e. the epithelial-mesenchymal transition (EMT). The EMT is a central mechanism of drug resistance in cancer. Under physiological conditions, the EMT is an integral part of tissue development and repair. In cancer, however, cell signaling networks that control the EMT are hijacked to promote tumor cell survival and metastasis. Acting as central nodes in most oncogenic signaling networks, protein kinases are commonly dysregulated in cancer. Despite known roles for certain kinases in phenotypic transition, there are no studies that comprehensively map EMT-associated kinase pathways.

Accordingly, despite the development of therapeutic agents for in oncology, there remains a need to identify and develop additional druggable targets for treating various cancers, including and especially HCC. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of reducing resistance in a cancer cell to a chemotherapeutic agent, comprising contacting the cell with an agent that inhibits the expression or function of an epithelial-mesenchymal transition (EMT)-associated kinase.

In some embodiments, the chemotherapeutic agent is kinase inhibitor. In some embodiments, the kinase inhibitor is selected from Table 1. In some embodiments, the kinase inhibitor is an inhibitor of a kinase selected from EGFR, SRC, c-MET, RAF, IGH1R, MEK1/2, PI3K, CHECK1/2, PLK1, CDK1/2, FGFR, mTOR, and AURK. In some embodiments, the kinase inhibitor is selected from sorafenib, regorafenib, lenvatinib, cabozantinib, dinaciclib, tezolizumab, ramucirumab, and becacizumab.

In some embodiments, the cancer cell is a hepatocellular carcinoma cell.

In some embodiments, the step of contacting the cell with the agent prevents or reverses transition of the cancer cell from an epithelial phenotype to a mesenchymal phenotype.

In some embodiments, the EMT-associated kinase is selected from the kinases listed in Table 2. In some embodiments, the EMT-associated kinase is selected from AXL, MET, EPHB2, FYN, AKT3, CAMK1D, NUAK1, NUAK2, EPHA4, CAMK1D, FYN, NEK3, CDK3, PLK1, CHEK1, EGFR, HIPK2, TNK2, LYN, PTK2, MAP3K12, MAPK9, MAPK8, FER, AAK1, CDK10, STK17B, and STK32B.

In some embodiments, the method further comprises contacting the cell with the chemotherapeutic agent.

In some embodiments, the cell is contacted in vivo in a subject with cancer, and the method comprises administering a therapeutically effective amount of the agent that inhibits the expression or function of the EMT-associated kinase.

In another aspect, the disclosure provides a method of treating cancer. The method can be characterized as a method of enhancing sensitivity of a cancer cell to a kinase inhibitor therapy in a subject in need thereof, comprising administering to the subject an effective amount of an agent that inhibits the expression or function of an epithelial-mesenchymal transition (EMT)-associated kinase.

In some embodiments, the method comprises administering a combination of effective amounts of the agent that inhibits the expression or function of an epithelial-mesenchymal transition (EMT)-associated kinase and a chemotherapeutic agent. In some embodiments, the chemotherapeutic agent is selected from the kinase inhibitors are listed in Table 1. In some embodiments, the agent inhibits an EMT-associated kinase disclosed in Table 2. In some embodiments, the agent inhibits an EMT-associated kinase is an agent selected the agents listed in Table 3.

In another aspect, the disclosure provides a method of profiling the kinome in a cell or population of similar cells.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1E: A pharmacoproteomics platform linking kinome features to drug response. (1A) Schematic of the kinome-centric pharmacoproteomics platform. (1B) Pearson's r-values for FGFR1-4 expression features correlated with the AUCs of all 22 FGFR KIs in our drug screen. (1C) Phosphosites in the FGFR-RAF/MEK/ERK-cell cycle signaling cascade correlated with responses to the seven FGFR KIs that showed the strongest correlation with FGFR3 and FGFR4 tyrosine phosphorylation (see (1B)). (1D) Examples for pathways that are associated with sensitivity (positive wNES) or resistance (negative wNES) to clinical HCC drugs. wNES is the FDR-weighted normalized Reactome pathway enrichment score. (1E) Correlation of pathway-based drug response signatures defined in 17 HCC lines with kinome pathway signatures identified in clinical HCC samples, showing that response signatures for specific KI drugs are enriched in human tumors.

FIGS. 2A-2D: The EMT state defines HCC drug resistance phenotypes and kinase signaling network activity. (2A) Clustering of mean wNES values for 34 representative Reactome pathways across the 299 drugs grouped into 11 classes with similar response pathway signatures (see FIG. 7A for complete Reactome pathway terms). (2B) Hierarchical clustering of EMT marker mRNA expression in the 17 CCLE HCC lines. (2C) Difference in cell motility between epithelial and mesenchymal HCC cells (wound healing assay, Student's T-test, P=0.02). 16 of the 17 CCLE HCC lines and the mesenchymal FOCUS line were tested. (2D) Kinases (circles, left) and their phosphorylation sites (right) significantly overexpressed in epithelial or mesenchymal HCC lines (BH-FDR=0.05). Kinases with a log 2 MS ratio >2 are labeled. The largest phosphosite ratio between EMT phenotypes for each kinase is plotted and kinases with activating phosphorylation sites are highlighted.

FIGS. 3A-3I: FZD2-AXL-NUAK1/2 signaling drives HCC cell EMT. (3A) Kinase protein (left) and phosphosite (right) expression differences in FOCUS AXL RNAi cells over WT cells overlaid on the human kinome dendrogram. The top 20 highest regulated kinases are labeled. The phosphorylation site with the highest absolute MS ratio on each kinase was plotted. (3B) Comparing AXL and FZD2 RNAi-dependent kinase expression changes in FOCUS cells and EMT-dependent kinase expression in the 17 HCC line panel (changes >4-fold). (3C) Log 2 MS ratios of the nine common RNAi- and EMT-dependent kinases (see (3B)). (3D) qPCR of AXL mRNA in FOCUS STATS RNAi cells. (3E) Wound healing assay in FOCUS AXL RNAi cells compared to control shRNA cells. (3F) Wound healing assay in FOCUS NUAK1 and NUAK2 RNAi cells compared to controls. (3G) Comparing the effect of NUAK1 RNAi, NUAK2 RNAi and AXL RNAi on kinase expression in FOCUS cells (changes >4-fold). (3H) Change of protein expression of AXL, NUAK1, and NUAK2 with three kinases (AKT1, SRC and MERTK) as internal controls upon knockdown of either of these kinases. (3I) qPCR of mRNA changes in EMT markers upon knockdown of either NUAK1 or NUAK2 in FOCUS cells.

FIGS. 4A-4I: Perturbation of AXL or NUAK1/2 function reverses the EMT and increases HCC cell sensitivity to cell cycle- and DDR kinase inhibitors. (4A) Activating phosphosites on CHEK1 and 2 and their substrates enriched in seven epithelial vs. ten mesenchymal HCC cell lines. (4B) Phosphorylation sites on CHEK1 and its substrates that indicate activation of this kinase in FOCUS AXL RNAi cells over WT and that are associated with EMT in the 17-cell line panel. (4C) Difference of mean AUC values in epithelial vs. mesenchymal HCC cells for the 299 KI panel for the 11 KI pathway clusters (see FIG. 7A). (4D) Strategy for reversing the EMT and increasing drug sensitivity through NUAK1/2 and AXL inhibition. (4E) and (4F) EC₅₀-curves of drug co-treatment experiments in SNU449 cells (n=4, error bars are S.D.). (4G) Heatmap of drug synergy and titratability of AZD7762 and WZ4003 in SNU449 cells. (4H) and (4I) Bar plots of drug treatment experiments with AZD7762 and dinaciclib in the SNU449 NUAK1/2 RNAi cell lines and FOCUS NUAK1/2 and AXL RNAi cell line, (n=4, error bars are the 95% confidence interval (CI)).

FIGS. 5A and 5B: Kinobead/LC-MS workflow and kinases, their phosphorylation sites and complex components identified in the 17 HCC cell line panel, and HTS drug screen results. Related to FIGS. 1A-1E. (5A) Kinobead/LC-MS workflow. (B) KI AUC values across the 17 HCC cell line panel. Low AUCs correspond to a strong drug response, i.e. more cell killing. Representative broadly active KIs (AURK, CDK, CHEK1/2, MEK1/2, MTOR, PLK1) are shown in the top panel. Representative KIs active only in specific cell lines (EGFR, FGFR, BRAF and IGF1R) are shown in the bottom panel. Cell lines were subjected to semi-supervised hierarchical clustering in Perseus (see ‘STAR Methods’), identifying a cluster of seven cell lines with high drug resistance and a cluster of 10 cell lines that are more sensitive to drug treatment.

FIG. 6: AUC-kinome feature correlation and kinome-GSEA. Related to FIGS. 1A-1E. Pearson's r-values for RAF kinase expression features (mRNA, protein and phosphopeptide) correlated with the AUCs of all 12 BRAF KIs contained in the drug screen (see ‘STAR Methods’). The clinical BRAF inhibitors sorafenib and regorafenib are highlighted.

FIGS. 7A-7C: Classification of KI drugs by pathway enrichment, EMT-Associated kinase activity, and AXL RNAi-induced EMT reversal and kinome rewiring. Related to FIGS. 2A-3I. (7A) Pairwise correlation of GSEA Reactome pathway scores (FDR-weighted NES, wNES) for 299 KIs; r-values are clustered into 11 groups of KIs by their similarity in enriched signaling pathways. Partial lists of primary targets (from literature) within KI clusters are shown. The complete list is available in the interactive web application (Lau, H.-T., et al. (2019). Kinome features, signaling pathways, and drug response in HCC (at quantbiology.org/hcckinome) (Ong Lab), incorporated herein by reference in its entirety). (7B) Activating phosphosites on kinases overexpressed in mesenchymal compared to epithelial cells. (7C) Activating phosphosites on important mitogenic and cell cycle kinases overexpressed in epithelial vs. mesenchymal cells.

FIGS. 8A-8C: FZD2 and NUAK1/2 RNAi-dependent kinome rewiring in FOCUS cells, and NUAK1 overexpression in C3A and SNU398 cells. Related to FIGS. 3A-3I. (8E) qPCR analysis of NUAK1 mRNA in C3A and SNU398 cells overexpressing NUAK1 plasmid DNA. (8F, 8G) Effect of NUAK1 overexpression (OE) on EMT markers in C3A and SNU398 cells, respectively.

FIGS. 9A-9E: EMT state-dependent activation of cell cycle and DNA damage response signaling. Related to FIGS. 4A-I. (9A) STRING interaction network of CDK1 and 2 substrates and corresponding CDK1/2 phosphorylation sites enriched in AXL RNAi FOCUS cells over WT. (9B) Difference in expression of known CDK2, MAPK1/3 and CHEK1/2 phospho-substrate sites (Phosphosite Plus) between epithelial and mesenchymal HCC cells. (9C) Difference in expression of known ATR and WEE1 phospho-substrate sites (Phosphosite Plus) between epithelial and mesenchymal HCC cells. (9D) Phosphorylation sites on WEE1 and its substrates that indicate activation of this kinase in FOCUS AXL RNAi cells over WT and that are associated with EMT in the 17-cell line panel. (9E) qPCR analysis of NUAK1 and NUAK2 mRNA in NUAK1 and 2 RNAi SNU449 cells, respectively.

FIGS. 10A-10D: Workflow approach and characterization of AAK1 signaling complex proteins in mesenchymal and epithelial HCC cells indicating a role of AAK1 functionality in EMT. (10A) General validation workflow to test the biological function of kinases and their interaction partners function in EMT and cancer therapy resistance. (10B) Composition of the of the AAK1 signaling complex in HCC cells as determined by kinobead/LC-MS kinome activity profiling. Proteins known to be part of oncogenic signaling pathways that may promote EMT are highlighted. These proteins were selected for detailed analysis. (10C) Difference in protein expression of AAK1 and its interaction partners RALBP1, REPS1, and REPS2 comparing 7 drug-sensitive epithelial-like and 10 drug-resistant mesenchymal-like HCC cell lines as determined by kinobead/LC-MS kinome activity profiling. All proteins shown: FDR<0.05, two sample t-test with Benjamini-Hochberg correction, n=7 and 10. (10D) Difference in protein expression of AAK1 and its interaction partners comparing four human HCC tumor tissue samples with paired normal adjacent liver tissue using our kinome kinobead/LC-MS kinome profiling technology (see Example 1). *: FDR<0.05, two sample t-test with Benjamini-Hochberg correction, all n=6.

FIGS. 11A-11F: Knockdown and drug synergy data validating role of AAK1 in EMT. (11A-11C) Results from qPCR analysis of three different mesenchymal HCC cell lines (FOCUS (11A), SKHep1 (11B), SNU761 (11C)) that have been stably transfected with a plasmid encoding shRNAs that specifically target AAK1 and its interaction partners or a scrambled shRNA (control). (11D-11F) Drug synergy of knockdown of AAK1 and its interaction partners RALPB1, REPS1 and REPS2 sensitizes therapy-resistant and mesenchymal-like HCC cells to treatment with targeted cancer drugs in three different HCC cell lines (FOCUS (11D), SKHep1 (11E), SNU761 (11F)). * p<0.05, ** p<0.01, *** p<0.005, **** p<0.001.

FIGS. 12A-12D: Analysis of the kinase CAMK1D according to the validation workflow illustrated in FIG. 10A. (12A) Difference in protein expression of EMT-associated kinases AKT3, AXL, CAMK1D, CDK10, EPHB2, NUAK1, NUAK2, STK17A, STK17B, and STK32B comparing 7 drug-sensitive epithelial-like and 7 drug-resistant mesenchymal-like HCC cell lines using the kinome kinobead/LC-MS kinome profiling technology. All proteins FDR<0.05, two sample t-test with Benjamini-Hochberg correction, n=7 and 10. (12B) Difference in protein expression of EMT-associated kinases, including CAMK1D, (see FIG. 10A) comparing four human HCC tumor tissue samples with paired normal adjacent liver tissue using our kinome kinobead/LC-MS kinome profiling technology (see Example 1). The results demonstrate that CAMK1D is overexpressed in at least 2/4 HCC patients' tumors. *: FDR<0.05, two sample t-test with Benjamini-Hochberg correction, n=6 each. (12C) Results from qPCR analysis of mesenchymal FOCUS, SNU449 and SNU761 cell lines that have been stable transfected with a plasmid encoding shRNAs that specifically target CAMK1D (see FIG. 10A) or a scramble shRNA (control). (12D) Kinobead/LC-MS kinome activity profiling of FOCUS, SNU449 and SNU761 CAMK1D KD cell lines.

FIGS. 13A-13D: Further analysis of the kinase CAMK1D according to the validation workflow illustrated in FIG. 10A. (13A) STRING pathway analysis using Reactome pathways of proteins differentially phosphorylated between FOCUS scramble shRNA control cells and FOCUS CAMK1D KD cells. (13B) Database alignment of CAMK1D-dependent phosphopeptide expression (see FIG. 10A) using the PhosphositesPlus functional phosphorylation site dataset. (13C) Database alignment of CAMK1D-dependent phosphopeptide expression (see FIG. 10A) using the PhosphositesPlus kinase-substrate relationship dataset. (13D) shRNAi knockdown of CAMK1D sensitizes mesenchymal HCC cells to treatment with targeted cancer drugs.

FIGS. 14A and 14B: Analysis of the kinases CDK10, STK32B, and STK17B according to the validation workflow illustrated in FIG. 10A. (14A) Results from qPCR analysis of mesenchymal FOCUS, SNU423, JHH6, and SKHep1 cell lines that have been stably transfected with a plasmid encoding shRNAs that specifically target CDK10, STK17B, or STK32B (see FIG. 10A) or a scramble shRNA (control). (14B) Kinobead/LC-MS kinome activity profiling of FOCUS, SNU423, JHH6 and SKHep1 cell lines in which CDK10, STK17B or STK32B have been depleted by shRNAi.

### DETAILED DESCRIPTION

The present disclosure is based on the inventors' investigation into the kinome of cancer cells that have undergone epithelial-mesenchymal transition (EMT) and are characteristically resistant to existing kinase-targeting therapies. The inventors posited that investigation of uncharted and dysregulated HCC kinase signaling could reveal new mechanisms of EMT-related drug resistance, molecular markers of drug response, and new drug targets. The initial work, described in more detail below, addressed a kinome-centric proteomics approach to study hepatocellular carcinoma (HCC) responses to kinase inhibitor drugs.

Briefly, quantifying the activity of kinase-dependent signaling networks requires measuring kinase expression levels, their post-translational modifications (PTMs), and their association with regulatory proteins. Kinase expression and phospho-activation can be assessed by mass spectrometry (MS)-based proteomics techniques such as global proteomics and phosphoproteomics, targeted MS analyses of kinase activation loop phosphorylation sites, and by kinobead kinase affinity enrichment coupled to phosphopeptide analyses (kinobead/LC-MS). Kinobead/LC-MS offers deep analytical coverage of kinases, their PTMs and kinase-regulatory proteins, quantifying kinome activity in a comprehensive and unbiased manner.

Consequently, the inventors implemented an enhanced kinobead/LC-MS approach to map features of kinome activity to growth inhibition caused by 299 kinase-targeted drugs across a panel of 17 HCC cell lines. A gene set enrichment analysis (GSEA) was applied to score 275 kinase-dependent cancer pathways with drug responses and to generate a comprehensive database of KI drug response signatures in HCC. Analyzing human HCC samples with kinobead/LC-MS, drug response signatures were quantified that could act as candidate predictive markers for personalized treatment. These analyses identified distinct signaling networks and drug response phenotypes closely linked to the EMT, and that the cellular EMT-state broadly impacts kinase expression and activation. In particular, a novel FZD2-AXL-NUAK1/2 signaling module was identified that promotes HCC cell EMT. Genetic knockdown or small-molecule inhibition of these proteins reversed the EMT, activated replication stress signaling, and increased sensitivity of HCC cells to drugs. It was demonstrated that unbiased kinome-centric pharmacoproteomics identifies molecular markers and signaling pathways underlying drug response, reveals novel kinases important for drug resistance, and suggests rational drug combinations for HCC treatment. This dataset of drug response signatures and EMT-associated signaling pathways is a valuable resource in functional studies of HCC cell signaling and is accessible through a web resource that allows real-time data interrogation and visualization.

In accordance with the foregoing, in one aspect the disclosure provides a method of reducing resistance in a cancer cell to a chemotherapeutic agent. The method comprises contacting the cell with an agent that inhibits the expression or function of an epithelial-mesenchymal transition (EMT)-associated kinase. The reduction in resistance can also be characterized as an increase of sensitivity of the cancer cell to the chemotherapeutic. In some embodiments, this effect can be established by an increase in cell death, reduction in cell expansion, reduction in cell motility, and the like, upon exposure to the same conditions (e.g., same concentration of the chemotherapeutic agent) but without contacting with the agent that inhibits the expression or function of an epithelial-mesenchymal transition (EMT)-associated kinase.

A broad class of chemotherapeutic agents encompassed by the disclosure is kinase inhibitors. As the name suggests, kinase inhibitors (KIs) target kinases in the cell. Kinases are a broad category of enzymes that play important roles in cell signaling, metabolism, division, survival, and the like. Some kinases are more active in cancer cells, considering the altered biology emphasizing growth and division. Accordingly, kinases represent a powerful class of targets for chemotherapeutic intervention. Representative kinase inhibitors encompassed by the disclosure, and their known targets, are listed in Table 1. As described in more detail herein, many cancers develop phenotypes that confer resistance to chemotherapeutic agents, such as kinase inhibitors, thus rendering such interventions less effective over time and allowing the cancer to remain and thrive. However, the exposure of the cancer cell to the agent that inhibits the expression or function of an epithelial-mesenchymal transition (EMT)-associated kinase decreases the cell's resistance to, or increases the cell's sensitivity to the chemotherapeutic agent. In some embodiments, the chemotherapeutic agent targets a kinase selected from EGFR, SRC, c-MET, RAF, IGH1R, MEK1/2, PI3K, CHECK1/2, PLK1, CDK1/2, FGFR, mTOR, and AURK. In some embodiments, the kinase inhibitor is selected from sorafenib, regorafenib, lenvatinib, cabozantinib, dinaciclib, tezolizumab, ramucirumab, and becacizumab.

TABLE 1
Exemplary kinase inhibitors (KI) and targets thereof.

| | | Target Groups (Total |
|---|---|---|
| Agent (Kinase Inhibitor) | Known Kinase Target(s) | #Members) |
| 3-Methyladenine | PIK3CG | PI3K (39) |
| 5-Iodotubercidin | MAPK3, PRKACA, ADK, | CK2 (5), MAPK14 (8), PRKC (10) |
| | CSNK1A1, CSNK2A1, | |
| | INSR, PRKC | |
| A66 | PIK3CA, PIK3CG, | PI3K (39) |
| | PIK3CD, PIK3CB | |
| A-674563 | AKT1, CDK2, PRKACA, | AKT (10), CDK (22), GSK3 (15), |
| | GSK3B, PRKCD, MAPK3 | PRKC (10) |
| A-769662 | PRKAA1, PRKAA2 | AMPK (2) |
| AEE788 (NVP-AEE788) | EGFR, ERBB2, FLT1, | ABL (21), EGFR (26), ERBB2 |
| | ERBB4, ABL1, SRC, KDR, | (16), VEGFR/KDR (65), SRC (14) |
| | CSF1R | |
| Afatinib (BIBW2992) | EGFR, ERBB2, ERBB4 | EGFR (26), ERBB2 (16) |
| AG-1024 | IGF1R, INSR | IGF1R (7) |
| AG-1478 (Tyrphostin AG- | EGFR | EGFR (26) |
| 1478) | | |
| AG-490 | EGFR | EGFR (26) |
| AMG 900 | AURKA, AURKB, | AURK (27), MAPK14 (8) |
| | AURKC, MAPK14 | |
| AMG-208 | MET | MET (21) |
| AMG458 | MET | MET (21) |
| Amuvatinib (MP-470) | KIT, PDGFRA, FLT3 | VEGFR/KDR (65), KIT (27), |
| | | PDGFR (28) |
| Apatinib (YN968D1) | KDR, RET | VEGFR/KDR (65) |
| ARQ 197 (Tivantinib) | MET | MET (21) |
| ARRY334543 | EGFR, ERBB2 | EGFR (26), ERBB2 (16) |
| Arry-380 | ERBB2 | ERBB2 (16) |
| AS-252424 | PIK3CA, PIK3CG, | CK2 (5), PI3K (39) |
| | CSNK2A1 | |
| AS-604850 | PIK3CG | PI3K (39) |
| AS-605240 | PIK3CA, PIK3CG, | PI3K (39) |
| | PIK3CD, PIK3CB | |
| AS703026 | MAP2K1, MAP2K2 | MEK1/2 (12) |
| AS-703026 (pimasertib) | MAP2K1, MAP2K2 | MEK1/2 (12) |
| AST-1306 | EGFR, ERBB2, ERBB4 | EGFR (26), ERBB2 (16) |
| AT7519 | CDK1, CDK2, CDK3, | CDK (22), GSK3 (15) |
| | CDK4, CDK6, CDK5, | |
| | CDK9, GSK3B | |
| AT7867 | AKT1, AKT2, AKT3, | AKT (10) |
| | RPS6KB1, RPS6KA1, | |
| | PRKACA | |
| AT9283 | ABL1, JAK2, AURKA, | ABL (21), AURK (27), FGFR |
| | AURKB, FGFR1, GSK3B, | (22), VEGFR/KDR (65), SRC |
| | FLT4, MERTK, RET, | (14), GSK3 (15), JAK (18), PDPK |
| | YES1, SRC, RPS6KA3, | (5) |
| | RPS6KA1, PDPK1, others | |
| Aurora A Inhibitor I | AURKA | AURK (27) |
| Axitinib | KDR, FLT1, FLT3, KIT, | VEGFR/KDR (65), KIT (27), |
| | PDGFRA, PDGFRB | PDGFR (28) |
| AZ 960 | JAK2 | JAK (18) |
| AZ628 | BRAF, RAF1 | RAF (12) |
| AZD2014 | mTOR | mTOR (28) |
| AZD4547 | FGFR1, FGFR2, FGFR3, | FGFR (22), VEGFR/KDR (65) |
| | KDR | |
| AZD5438 | CDK1, CDK2, CDK9 | CDK (22) |
| AZD6244 (Selumetinib) | MAP2K1, MAP2K2 | MEK1/2 (12) |
| AZD7762 | CHEK1, CHEK2 | CHEK1/2 (7) |
| AZD8055 | mTOR | mTOR (28) |
| AZD8330 | MAP2K1, MAP2K2 | MEK1/2 (12) |
| AZD8931 | EGFR, ERBB2, ERBB3 | EGFR (26), ERBB2 (16) |
| Barasertib (AZD1152-HQPA) | AURKB | AURK (27) |
| Baricitinib (LY3009104) | JAK1, JAK2, JAK3, TYK2 | JAK (18) |
| BAY 11-7082 | TNFα induced phos of IkB-α | NF-kB Kinase (5) |
| BAY 11-7085 | inhibitor of IkB-α phosphor | NF-kB Kinase (5) |
| BEZ235 (NVP-BEZ235) | MTOR, PIK3CA, PIK3CG, | ATM/ATR (6), mTOR (28), PI3K |
| | PIK3CD, ATR | (39) |
| BGJ398 (NVP-BGJ398) | FGFR1, FGFR2, FGFR3, | FGFR (22) |
| | FGFR4 | |
| BI 2536 | PLK1, PLK2, PLK3 | PLK1 (7) |
| BI-2536 | PLK1, PLK2, PLK3 | PLK1 (7) |
| BI6727 (Volasertib) | PLK1 | PLK1 (7) |
| BIBF1120 (Vargatef) | KDR, FLT1, FLT3, | FGFR (22), VEGFR/KDR (65), |
| | PDGFRB, PDGFRA, | SRC (14), PDGFR (28) |
| | FGFR2, FGFR3, FGFR2, | |
| | FGFR4, SRC, LYN | |
| BIRB 796 (Doramapimod) | MAPK14 | MAPK14 (8) |
| BIX 02188 | MAP2K5 | MAP2K5 (2) |
| BIX 02189 | MAP2K5 | MAP2K5 (2) |
| BKM120 (NVP-BKM120) | PIK3CA, PIK3CG, | PI3K (39) |
| | PIK3CD | |
| BML 277 | CHEK2 | CHEK1/2 (7) |
| BMS 777607 | MET, AXL, MST1R, | AURK (27), VEGFR/KDR (65), |
| | TYRO3, MERTK, FLT3, | MET (21) |
| | AURKB | |
| BMS 794833 | MET, KDR | VEGFR/KDR (65), MET (21) |
| BMS-265246 | CDK1, CDK2, CDK4 | CDK (22) |
| BMS-599626 (AC480) | EGFR, ERBB2, ERBB4 | EGFR (26), ERBB2 (16) |
| BMS-754807 | IGF1R, INSR, MET, | AURK (27), VEGFR/KDR (65), |
| | AURKA, AURKB, | IGF1R (7), MET (21) |
| | MST1R, FLT3, NTRK1, | |
| | NTRK2 | |
| Bosutinib (SKI-606) | SRC, ABL1 | ABL (21) |
| Brivanib (BMS-540215) | KDR, FLT1, FGFR1 | FGFR (22), VEGFR/KDR (65) |
| Brivanib alaninate (BMS- | KDR, FGFR1, FLT1 | FGFR (22), VEGFR/KDR (65) |
| 582664) | | |
| BS-181 HCl | CDK7 | CDK (22) |
| BX-795 | PDPK1, KIT, CDK2 | CDK (22), KIT (27), PDPK (5) |
| BX-912 | PDPK1, PRKACA, KDR | VEGFR/KDR (65), PDPK (5) |
| BYL719 | PIK3CA | PI3K (39) |
| C3742 | CHEK2 | CHEK1/2 (7) |
| CAL-101 (GS-1101) | PIK3CG, PIK3CD | PI3K (39) |
| CAY10505 | PIK3CG | PI3K (39) |
| CCT128930 | AKT2, PRKACA, | AKT (10) |
| | RPS6KB1 | |
| CCT129202 | AURKA, AURKB, | AURK (27) |
| | AURKC | |
| CCT137690 | AURKA, AURKB, | AURK (27) |
| | AURKC | |
| Cediranib (AZD2171) | KDR, FLT1, FLT3, | FGFR (22), VEGFR/KDR (65), |
| | FGFR1, KIT, PDGFRA, | KIT (27), PDGFR (28) |
| | PDGFRB | |
| CEP33779 | JAK2 | JAK (18) |
| CH5424802 | ALK | ALK (6) |
| CHIR-124 | CHEK1, FLT3, PDGFRB, | CHEK1/2 (7), VEGFR/KDR (65), |
| | GSK3B | GSK3 (15), PDGFR (28) |
| CHIR-98014 | GSK3A, GSK3B | GSK3 (15) |
| CI-1033 (Canertinib) | EGFR, ERBB2 | EGFR (26), ERBB2 (16) |
| CI-1040 (PD184352) | MAP2K1, MAP2K2 | MEK1/2 (12) |
| CP 673451 | PDGFRB, PDGFRA | PDGFR (28) |
| CP-724714 | ERBB2 | ERBB2 (16) |
| Crenolanib (CP-868596) | PDGFRB, PDGFRA | PDGFR (28) |
| Crizotinib (PF-02341066) | MET, ALK | ALK (6), MET (21) |
| cx-4945 (Silmitasertib) | CSNK2A1 | CK2 (5) |
| CYC116 | AURKA, AURKB, FLT3, | AURK (27), CDK (22), |
| | CDK2, CDK9, RPS6KB1, | VEGFR/KDR (65) |
| | KDR | |
| Cyt387 | JAK1, JAK2, JAK3 | JAK (18) |
| Dabrafenib (GSK2118436) | BRAF, RAF1 | RAF (12) |
| Dacomitinib (PF299804,PF- | EGFR, ERBB2, ERBB4 | EGFR (26), ERBB2 (16) |
| 00299804) | | |
| Danusertib (PHA-739358) | AURKA, AURKB, | ABL (21), AURK (27), FGFR (22) |
| | AURKC, FGFR1, ABL1, | |
| | RET, SRC | |
| Dasatinib (BMS-354825) | ABL1, SRC, KIT | ABL (21), KIT (27) |
| DCC-2036 (Rebastinib) | ABL1, FLT3, KDR, TEK, | ABL (21), VEGFR/KDR (65), |
| | LYN, SRC, FGR, others | SRC (14), TEK (8) |
| Deforolimus (Ridaforolimus) | mTOR | mTOR (28) |
| Desmethyl Erlotinib (CP- | EGFR | EGFR (26) |
| 473420) | | |
| Dinaciclib (SCH727965) | CDK1, CDK2, CDK5, | CDK (22) |
| | CDK9 | |
| Dovitinib (TKI-258) | FLT3, KIT, FGFR3, FLT1, | FGFR (22), VEGFR/KDR (65), |
| | KDR, PDGFRB, CSF1R | KIT (27), PDGFR (28) |
| Dovitinib Dilactic acid | FLT3, KIT, FGFR3, FLT1, | FGFR (22), VEGFR/KDR (65), |
| (TKI258 Dilactic acid) | KDR, PDGFRB, CSF1R | KIT (27), PDGFR (28) |
| E7080 (Lenvatinib) | KDR, FLT1, FLT3, | FGFR (22), VEGFR/KDR (65), |
| | PDGFRB, PDGFRA, KIT, | KIT (27), PDGFR (28) |
| | FGFR1 | |
| EMD-1214063 | MET | MET (21) |
| ENMD-2076 | FLT3, FLT4, AURKA, | ABL (21), AURK (27), FGFR |
| | AURKB, KDR, SRC, LCK, | (22), VEGFR/KDR (65), SRC |
| | PTK2, FGFR1, ABL1, | (14), JAK (18), KIT (27), PTK2 |
| | FYN, YES1, FGFR1, | (5) |
| | FGFR2, JAK2, KIT | |
| Enzastaurin (LY317615) | PRKCD, PRKCB, PRKCE, | PRKC (10) |
| | PRKCG | |
| Erlotinib HCl | EGFR | EGFR (26) |
| Everolimus (RAD001) | mTOR | mTOR (28) |
| Flavopiridol hydrochloride | CDK1, CDK2, CDK4, | CDK (22) |
| | CDK6, CDK7 | |
| Foretinib (GSK1363089, | MET, FLT1, FLT3, KDR, | VEGFR/KDR (65), KIT (27), |
| XL880) | MST1R, MERTK, TEK, | MET (21), PDGFR (28), TEK (8) |
| | KIT, PDGFRB | |
| FRAX597 (PAKi) | PAK1, PAK2, PAK3 | PAK (2) |
| GDC-0068 | AKT1, AKT2, AKT3 | AKT (10) |
| GDC-0879 | BRAF | RAF (12) |
| GDC-0941 | MTOR, PIK3CA, PIK3CG, | mTOR (28), PI3K (39) |
| | PIK3CD | |
| GDC-0980 (RG7422) | MTOR, PIK3CA, PIK3CG, | mTOR (28), PI3K (39) |
| | PIK3CD | |
| Gefitinib (Iressa) | EGFR | EGFR (26) |
| GF109203X | PRKCD, PRKCB, PRKCE, | PRKC (10) |
| | PRKCG | |
| Golvatinib (E7050) | MET, KDR | VEGFR/KDR (65), MET (21) |
| GP29 | ABL1, SRC | ABL (21) |
| GSK1059615 | PIK3CA, PIK3CG, | mTOR (28), PI3K (39) |
| | PIK3CD, PIK3CB, mTOR | |
| GSK1070916 | AURKB, AURKC, SIK1, | AURK (27), FGFR (22), |
| | FLT1, FLT4, FGFR1, TEK | VEGFR/KDR (65), TEK (8) |
| GSK1120212 (Trametinib) | MAP2K1, MAP2K2 | MEK1/2 (12) |
| GSK1838705A | IGF1R, ALK, INSR | ALK (6), IGF1R (7) |
| GSK1904529A | IGF1R, INSR | IGF1R (7) |
| GSK2126458 | PIK3CA, PIK3CG, | mTOR (28), PI3K (39) |
| | PIK3CD, PIK3CB, mTOR | |
| GSK461364 | PLK1 | PLK1 (7) |
| GSK690693 | AKT1, AKT2, AKT3, | AKT (10), PRKC (10) |
| | PRKCH, PRKCQ, PRKCD, | |
| | PRKX, PRKCB, PRKCE, | |
| | PRKG, PRKACA | |
| Hesperadin | AURKA, AURKB | AURK (27) |
| HMN-214 | PLK1 | PLK1 (7) |
| IC-87114 | PIK3CG, PIK3CD | PI3K (39) |
| Imatinib (Gleevec) | PDGFRB, ABL1, KIT | ABL (21), KIT (27), PDGFR (28) |
| Imatinib Mesylate | PDGFRB, ABL1, KIT | ABL (21), KIT (27), PDGFR (28) |
| IMD 0354 | IKBKB | NF-kB Kinase (5) |
| INCB28060 | MET | MET (21) |
| Indirubin | GSK3B | GSK3 (15) |
| INK 128 | MTOR, PIK3CA, PIK3CG, | mTOR (28), PI3K (39) |
| | PIK3CD | |
| JNJ-38877605 | MET | MET (21) |
| JNJ-7706621 | CDK1, CDK2, CDK3, | AURK (27), CDK (22), FGFR |
| | CDK4, CDK6, AURKA, | (22), VEGFR/KDR (65), GSK3 |
| | AURKB, KDR, FGFR2, | (15), TEK (8) |
| | TEK, GSK3B | |
| Ki8751 | KDR, KIT, PDGFRA | VEGFR/KDR (65), KIT (27), |
| | | PDGFR (28) |
| KRN 633 | KDR, FLT1, FLT3 | VEGFR/KDR (65) |
| Ku-0063794 | mTOR | mTOR (28) |
| KU-55933 | ATM | ATM/ATR (6) |
| KU-60019 | ATM | ATM/ATR (6) |
| KW 2449 | FLT3, ABL1, AURKA, | ABL (21), AURK (27), FGFR |
| | FGFR1, JAK2, KIT, SRC | (22), VEGFR/KDR (65), SRC |
| | | (14), JAK (18), KIT (27) |
| KX2-391 | SRC | SRC (14) |
| Lapatinib Ditosylate (Tykerb) | EGFR, ERBB2 | EGFR (26), ERBB2 (16) |
| LDN193189 | ALK2, BMPR1A | BMP/TGF-b/Activin Receptors (3) |
| Linifanib (ABT-869) | PDGFRB, KDR, KIT, | VEGFR/KDR (65), KIT (27), |
| | CSF1R, TEK, FLT1, FLT4 | PDGFR (28), TEK (8) |
| Linsitinib (OSI-906) | IGF1R, INSR, INSRR | IGF1R (7) |
| LY2228820 | MAPK14 | MAPK14 (8) |
| LY2603618 (IC-83) | CHEK1 | CHEK1/2 (7) |
| LY2784544 | JAK1, JAK2, JAK3, FLT3, | ALK (6), AURK (27), FGFR (22), |
| | FLT4, FGFR2, FGFR3, | VEGFR/KDR (65), JAK (18) |
| | TYK2, NTRK1, KDR, | |
| | ALK, MUSK, AURKA, | |
| | MAP3K9 | |
| LY294002 | PIK3CA | PI3K (39) |
| Masitinib (AB1010) | KIT | KIT (27) |
| MGCD-265 | MET, KDR, FLT1, FLT3, | VEGFR/KDR (65), MET (21), |
| | TEK, MST1R | TEK (8) |
| MK2206 | AKT1, AKT2, AKT3 | AKT (10) |
| MK-2206 dihydrochloride | AKT1, AKT2, AKT3 | AKT (10) |
| MK-2461 | MET, MST1R, FLT1, | FGFR (22), VEGFR/KDR (65), |
| | MERTK, FGFR2, FGFR3, | JAK (18), MET (21) |
| | JAK2, KDR | |
| MK-5108 (VX-689) | AURKA | AURK (27) |
| MLN8054 | AURKA, AURKB | AURK (27) |
| MLN8237 (Alisertib) | AURKA | AURK (27) |
| Motesanib Diphosphate | KDR, FLT1, FLT3, | VEGFR/KDR (65), KIT (27), |
| | PDGFRB, PDGFRA, KIT, | PDGFR (28) |
| | RET | |
| Mubritinib (TAK 165) | ERBB2 | ERBB2 (16) |
| Neratinib (HKI-272) | ERBB2, EGFR | EGFR (26), ERBB2 (16) |
| Nilotinib (AMN-107) | ABL1 | ABL (21) |
| NU7441(KU-57788) | PRKDC | PRKDC (10) |
| NVP-ADW742 | IGF1R | IGF1R (7) |
| NVP-BGT226 | MTOR, PIK3CA, PIK3CG, | mTOR (28), PI3K (39) |
| | PIK3CD | |
| NVP-BHG712 | EPHB4, RAF1, SRC, | ABL (21), RAF (12) |
| | ABL1 | |
| NVP-BSK805 | JAK1, JAK2, JAK3, TYK2 | JAK (18) |
| NVP-BVU972 | MET | MET (21) |
| NVP-TAE226 | PTK2, PTK2B, INSR, | VEGFR/KDR (65), IGF1R (7), |
| | IGF1R, MET, FLT4 | MET (21), PTK2 (5) |
| ON 01910.Na (Rigosertib) | PLK1, PDGFRB, FLT1, | ABL (21), CDK (22), |
| | ABL1, FYN, SRC, CDK1 | VEGFR/KDR (65), SRC (14), |
| | | PDGFR (28), PLK1 (7) |
| ON-01910(Rigosertib) | PLK1, PDGFRB, FLT1, | ABL (21), CDK (22), |
| | ABL1, FYN, SRC, CDK1 | VEGFR/KDR (65), SRC (14), |
| | | PDGFR (28), PLK1 (7) |
| OSI-027 | mTOR, PIK3CG | mTOR (28), PI3K (39) |
| OSI-420 (Desmethyl | EGFR | EGFR (26) |
| Erlotinib) | | |
| OSI-930 | KIT, LCK, RAF1, FLT1, | RAF (12), VEGFR/KDR (65), KIT |
| | KDR, CSF1R | (27) |
| OSU-03012 | PDPK1 | PDPK (5) |
| Otava 0107830108 | CSNK2A1 | CK2 (5) |
| Otava 1112092 | CDK4, CDK6 | CDK (22) |
| Otava 7015980251 | CSNK2A1 | CK2 (5) |
| Otava 7020402324 | IKBKE | NF-kB Kinase (5) |
| Otava 7070707035 | CDK4 | CDK (22) |
| Palomid 529 | MTOR | mTOR (28) |
| Pazopanib HCl | KDR, FLT1, FLT3, | FGFR (22), VEGFR/KDR (65), |
| | PDGFRB, PDGFRA, KIT, | KIT (27), PDGFR (28) |
| | FGFR1, CSF1R | |
| PCI-32765 (Ibrutinib) | SRC, EGFR, BTK, CSK, | EGFR (26), ERBB2 (16), SRC |
| | FGR, YES1, BMX, HCK, | (14), JAK (18) |
| | ERBB2, ITK, JAK3, FRK, | |
| | LCK, RET | |
| PD 0332991 (Palbociclib) HCl | CDK4, CDK6 | CDK (22) |
| PD0325901 | MAP2K1, MAP2K2 | MEK1/2 (12) |
| PD153035 HCl | EGFR | EGFR (26) |
| PD173074 | FGFR1, KDR | FGFR (22), VEGFR/KDR (65) |
| PD318088 | MAP2K1, MAP2K2 | MEK1/2 (12) |
| PD98059 | MAP2K1, MAP2K2 | MEK1/2 (12) |
| Pelitinib (EKB-569) | EGFR, SRC | EGFR (26) |
| PF-00562271 | PTK2, PTK2B, CDK1, | AURK (27), CDK (22), |
| | CDK2, CDK3, CDK5, | VEGFR/KDR (65), GSK3 (15), |
| | FLT3, GSK3A, GSK3B, | PTK2 (5) |
| | AURKA | |
| PF-03814735 | AURKA, AURKB, FLT1, | AURK (27), VEGFR/KDR (65), |
| | PTK2, NTRK1 | PTK2 (5) |
| PF-04217903 | MET | MET (21) |
| PF-04691502 | MTOR, PIK3CA, PIK3CG, | mTOR (28), PI3K (39) |
| | PIK3CD | |
| PF-05212384 (PKI-587) | mTOR, PIK3CA, PIK3CG | mTOR (28), PI3K (39) |
| PF-3758309 (PAKi) | PAK1, PAK2, PAK3, | PAK (2) |
| | PAK4, PAK5, PAK6 | |
| PH-797804 | MAPK14, MAPK13 | MAPK14 (8) |
| PHA-665752 | MET, MST1R, KDR | VEGFR/KDR (65), MET (21) |
| PHA-680632 | AURKA, AURKB, | AURK (27), FGFR (22) |
| | AURKC, FGFR1 | |
| PHA-767491 | CDC7, CDK1, CDK2, | CDK (22), GSK3 (15) |
| | CDK9, GSK3B | |
| PHA-793887 | CDK1, CDK2, CDK5, | CDK (22), GSK3 (15) |
| | CDK7, CDK9, GSK3B | |
| PHA-848125 | CDK1, CDK2, CDK4, | CDK (22) |
| | CDK5, CDK7, NTRK1 | |
| Phenformin hydrochloride | PRKAA1, PRKAA2 | AMPK (2) |
| PHPS1 | PTP Inhibitor V | PTP Inhibitor V (1) |
| PHT-427 | AKT1, PDPK1 | AKT (10), PDPK (5) |
| PI-103 | PRKDC, PIK3CA, | mTOR (28), PI3K (39), PRKDC |
| | PIK3CD, MTOR | (10) |
| Piceatannol | SYK | SYK (6) |
| PIK-293 | PIK3CG, PIK3CD | PI3K (39) |
| PIK-294 | PIK3CG, PIK3CD, | PI3K (39) |
| | PIK3CB | |
| PIK-75 | PRKDC, PIK3CA, | PI3K (39), PRKDC (10) |
| | PIK3CG, PIK3CD | |
| PIK-90 | PIK3CA, PIK3CG, | PI3K (39) |
| | PIK3CD | |
| PIK-93 | PIK3CA, PIK3CG, | ATM/ATR (6), PI3K (39), |
| | PIK3CD, PIK3CB, | PRKDC (10) |
| | PRKDC, ATM | |
| PKC412 (Midostaurin) | PRKCD, PRKCB, PRKCE, | VEGFR/KDR (65), PRKC (10), |
| | PRKCG, PRKACA, SYK, | SYK (6) |
| | KDR | |
| PKI-402 | MTOR, PIK3CA, PIK3CG, | mTOR (28), PI3K (39) |
| | PIK3CD | |
| Pluripotin (PT) | SRC, ABL1 | ABL (21) |
| PLX-4720 | BRAF, RAF1, PTK6 | RAF (12) |
| Ponatinib (AP24534) | ABL1, KDR, FGFR1, | ABL (21), FGFR (22), |
| | PDGFRA, SRC, KIT | VEGFR/KDR (65), SRC (14), KIT |
| | | (27), PDGFR (28), |
| PP-121 | PRKDC, MTOR, PDGFRB, | ABL (21), VEGFR/KDR (65), |
| | KDR, SRC, ABL1, HCK, | SRC (14), mTOR (28), PDGFR |
| | EPHB4, PIK3CA, PIK3CD | (28), PI3K (39), PRKDC (10) |
| PP242 | mTOR, PIK3CG, PRKDC | mTOR (28), PI3K (39), PRKDC |
| | | (10) |
| Quercetin (Sophoretin) | PIK3CG, PIK3CD, | PI3K (39), PRKC (10) |
| | PIK3CB, SRC, PRKC | |
| Quizartinib (AC220) | FLT3 | VEGFR/KDR (65) |
| R406 | SYK, FLT3 | VEGFR/KDR (65), SYK (6) |
| R406(free base) | SYK, FLT3 | VEGFR/KDR (65), SYK (6) |
| R788 (Fostamatinib) | SYK | SYK (6) |
| R935788 (Fostamatinib | SYK | SYK (6) |
| disodium, R788 disodium) | | |
| Raf265 derivative | RAF1, KDR | RAF (12), VEGFR/KDR (65) |
| Rapamycin (Sirolimus) | mTOR | mTOR (28) |
| RDEA-119 (BAY 869766) | MAP2K1, MAP2K2 | MEK1/2 (12) |
| Regorafenib (BAY 73-4506) | KIT, RAF1, BRAF, KDR, | RAF (12), VEGFR/KDR (65), KIT |
| | FLT1, FLT4, PDGFRB | (27), PDGFR (28) |
| Roscovitine (Seliciclib, | CDK1, CDK2, CDK5 | CDK (22) |
| CYC202) | | |
| Ruxolitinib (INCB018424) | JAK1, JAK2 | JAK (18) |
| SAR131675 | FLT3 | VEGFR/KDR (65) |
| Saracatinib (AZD0530) | SRC, ABL1, EGFR, | ABL (21), EGFR (26), |
| | EPHA2, KDR, LCK, FYN, | VEGFR/KDR (65), SRC (14), KIT |
| | LYN, KIT, YES1, BLK | (27) |
| SB 202190 | MAPK14, MAPK13 | MAPK14 (8) |
| SB 203580 | MAPK14 | MAPK14 (8) |
| SB 216763 | GSK3A, GSK3B | GSK3 (15) |
| SB 218078 | CHEK1 | CHEK1/2 (7) |
| SB 415286 | GSK3A, GSK3B | GSK3 (15) |
| SB 431542 | ACVR1B, TGFBR1, | BMP/TGF-b/Activin Receptors (3) |
| | ACVR1C | |
| SB 525334 | TGFBR1 | BMP/TGF-b/Activin Receptors (3) |
| SB590885 | BRAF, RAF1 | RAF (12) |
| Semaxanib (SU5416) | KDR | VEGFR/KDR (65) |
| SGI-1776 | PIM1, PIM2, PIM3, FLT3 | VEGFR/KDR (65), PIM (1) |
| SGX-523 | MET | MET (21) |
| SNS-032 (BMS-387032) | CDK2, CDK5, CDK7, | CDK (22), GSK3 (15) |
| | CDK9, GSK3B | |
| SNS-314 | AURKA, AURKB, | AURK (27) |
| | AURKC | |
| Sorafenib (Nexavar) | FLT1, PDGFRB, RAF1, | RAF (12), VEGFR/KDR (65), KIT |
| | BRAF, KDR, KIT | (27), PDGFR (28) |
| Sotrastaurin (AEB071) | PRKCD, PRKCB, PRKCE, | PRKC (10) |
| | PRKCG, PRKCQ, PRKCH | |
| SP600125 | MAPK8, MAPK9, | AKT (10), AURK (27), PRKC |
| | MAPK10, AURKA, | (10) |
| | NTRK1, MAP2K4, | |
| | MAP2K6, AKT1, | |
| | MAP2K3, PRKCA | |
| Staurosporine | PRKCD, PRKCB, PRKCE, | PRKC (10) |
| | PRKCG, others | |
| SU11274 | MET | MET (21) |
| Sunitinib Malate (Sutent) | KIT, FLT3, PDGFRB, | VEGFR/KDR (65), KIT (27), |
| | KDR | PDGFR (28) |
| TAE684 (NVP-TAE684) | ALK | ALK (6) |
| TAK-285 | EGFR, ERBB2, ERBB4 | EGFR (26), ERBB2 (16) |
| TAK-733 | MAP2K1, MAP2K2 | MEK1/2 (12) |
| TAK-901 | AURKA, AURKB, SRC, | AURK (27), CDK (22), CHEK1/2 |
| | AXL, FGFR1, JAK2, | (7), FGFR (22), JAK (18), PTK2 |
| | PTK2, CHEK2, CDK7 | (5) |
| | others | |
| Tandutinib (MLN518) | FLT3, KIT, PDGFRB, | VEGFR/KDR (65), KIT (27), |
| | CSF1R | PDGFR (28) |
| Telatinib (BAY 57-9352) | KIT, KDR, FLT3, | VEGFR/KDR (65), KIT (27), |
| | PDGFRA | PDGFR (28) |
| Temsirolimus (Torisel) | mTOR | mTOR (28) |
| TG 100713 | PIK3CA, PIK3CG, | PI3K (39) |
| | PIK3CD, PIK3CB | |
| TG100-115 | PIK3CA, PIK3CG, | PI3K (39) |
| | PIK3CD, PIK3CB | |
| TG101209 | FLT3, JAK2, JAK3, RET | VEGFR/KDR (65), JAK (18) |
| TG101348 (SAR302503) | JAK2, FLT3, RET | VEGFR/KDR (65), JAK (18) |
| TGX-221 | PIK3CB, PIK3CG | PI3K (39) |
| Thiazovivin | ROCK1, ROCK2 | ROCK (2) |
| Tideglusib | GSK3B | GSK3 (15) |
| Tie2 kinase inhibitor | TEK | TEK (8) |
| Tivozanib (AV-951) | FLT1, KDR, KIT, FLT3, | VEGFR/KDR (65), KIT (27), |
| | PDGFRB, PDGFRA, | PDGFR (28), TEK (8) |
| | EPHB4, TEK | |
| Tofacitinib (CP-690550, | JAK2, JAK3 | JAK (18) |
| Tasocitinib) | | |
| Tofacitinib citrate (CP-690550 | JAK2, JAK3 | JAK (18) |
| citrate) | | |
| Torin 1 | mTOR, PRKDC, PIK3CG | mTOR (28), PI3K (39), PRKDC |
| | | (10) |
| Torin 2 | mTOR, ATM, ATR, | ATM/ATR (6), mTOR (28), |
| | PRKDC | PRKDC (10) |
| TPCA-1 | IKBKB | NF-kB Kinase (5) |
| Triciribine (Triciribine | AKT1, AKT2 | AKT (10) |
| phosphate) | | |
| TSU-68 | PDGFRB | PDGFR (28) |
| TWS119 | GSK3B | GSK3 (15) |
| Tyrphostin AG 879 (AG 879) | ERBB2 | ERBB2 (16) |
| U0126-EtOH | MAP2K1, MAP2K2 | MEK1/2 (12) |
| Vandetanib (Zactima) | EGFR, KDR, FLT1 | EGFR (26), VEGFR/KDR (65) |
| Vatalanib dihydrochloride | KDR, FLT1 | VEGFR/KDR (65) |
| (PTK787) | | |
| Vemurafenib (PLX4032) | BRAF, RAF1, MAP4K5, | RAF (12) |
| | TNK2, FGR, LCK, NEK11, | |
| | PTK6 | |
| VX-680 (MK-0457, | AURKA, AURKB, | ABL (21), AURK (27), |
| Tozasertib) | AURKC, FLT3, ABL1 | VEGFR/KDR (65) |
| VX-702 | MAPK14 | MAPK14 (8) |
| WAY-600 | mTOR | mTOR (28) |
| WHI-P154 | EGFR, KDR, SRC | EGFR (26), VEGFR/KDR (65), |
| | | SRC (14) |
| Wortmannin | PIK3CA, PIK3CG, | ATM/ATR (6), PI3K (39), |
| | PIK3CD, PRKDC, ATM, | PRKDC (10) |
| | MYLK | |
| WP1066 | JAK2 | JAK (18) |
| WP1130 | ABL1 | ABL (21) |
| WYE-125132 | mTOR | mTOR (28) |
| WYE-354 | mTOR | mTOR (28) |
| WYE-687 | mTOR | mTOR (28) |
| WZ3146 | EGFR | EGFR (26) |
| WZ4002 | EGFR | EGFR (26) |
| WZ8040 | EGFR | EGFR (26) |
| XL147 | PIK3CA, PIK3CG, | PI3K (39) |
| | PIK3CD, PIK3CB | |
| XL-184 free base | KDR, MET | VEGFR/KDR (65), MET (21) |
| (Cabozantinib) | | |
| XL765 | PIK3CA, PIK3CG, | PI3K (39), PRKDC (10) |
| | PIK3CD, PIK3CB, PRKDC | |
| Y-27632 2HCl | ROCK1, ROCK2 | ROCK (2) |
| YM201636 | PIKFYVE | PIKFYVE (1) |
| ZM 336372 | RAF1 | RAF (12) |
| ZM-447439 | AURKA, AURKB | AURK (27) |
| ZSTK474 | PIK3CA, PIK3CG, | PI3K (39) |
| | PIK3CD | |

The method is applicable to any cancer that, for example, exhibits resistance to kinase inhibitor therapy. In some embodiments, the cancer cell is a carcinoma cancer cell. Hepatocellular carcinoma (HCC) is an exemplary cancer type encompassed by the present disclosure. While HCC is responsible for a large proportion of cancer deaths, there are relatively few druggable targets, limiting treatment options. Commonly used chemotherapeutic agents used for HCC are KIs, although rates of eventual resistance are relatively high, thus limiting the long term efficacy of such interventions. Accordingly, the disclosed method is particularly useful to re-invigorate and extend such therapies by counteracting such resistance and maintaining or increasing sensitivity of the cancer cell to the chemotherapeutic agent.

As described above, the transition of cancer cells from “epithelial” phenotype to a “mesenchymal” phenotype, referred to as epithelial-mesenchymal transition (EMT), is a critical mechanism underlying drug resistance in cancers. Critically, cancer cells with epithelial phenotypes are typically more sensitive to chemotherapy, such as therapies with kinase inhibitors, whereas cancer cells that develop mesenchymal phenotypes exhibit increased resistance to chemotherapy. As demonstrated below, this is due at least to some extent to a dysregulation of kinase signaling pathways that reorganizes the cell's reliance on various kinases and their targets to promote growth, division, and other survival functions. Accordingly, in some embodiments, the method (i.e., contacting the cell with the agent) prevents the cancer cell from transitioning from an epithelial phenotype to a mesenchymal phenotype. In some embodiments, the method promotes transition from a mesenchymal phenotype to an epithelial phenotype.

As described in more detail below, the inventors employed an analytical workflow to specifically assess the kinomes of epithelial-type and mesenchymal-type cancer cell lines and determined critical differences in kinome organization. Table 2 discloses kinases discovered to have either differential expression or differential activation features between mesenchymal and epithelial cells. These represent critical therapeutic targets to prevent or reverse the EMT and, thus, enhance sensitivity (or reduce resistance) to chemotherapeutic intervention. Accordingly, the kinases disclosed in Table 2 are encompassed by the present disclosure as embodiments of the epithelial-mesenchymal transition (EMT)-associated kinase that are targeted to reduce resistance to chemotherapeutic agents.

TABLE 2
Kinases with differential expression or activation features between mesenchymal and epithelial HCC cells.

| | | Log2 MS | EMT- |
|---|---|---|---|
| | | Intensity | Associated |
| Gene | | Ratio | Expression |
| Name | Protein Name | Mes./Epi. | Feature |
| AAK1 | AP2-associated protein kinase 1 | 2.13 | Protein |
| ABL1 | Tyrosine-protein kinase ABL1 | 1.06 | Phosphorylation |
| ABL2 | Abelson tyrosine-protein kinase 2 | 2.03 | Protein |
| ACVR1 | Activin receptor type-1 | 0.91 | Protein |
| AKT1 | RAC-alpha serine/threonine-protein kinase | 0.92 | Protein |
| AKT3 | RAC-gamma serine/threonine-protein kinase | 4.32 | Protein, |
| | | | Phosphorylation |
| AXL | Tyrosine-protein kinase receptor UFO | 7.17 | Protein, |
| | | | Phosphorylation |
| BMP2K | BMP-2-inducible protein kinase | 1.31 | Protein |
| BMPR2 | Bone morphogenetic protein receptor type-2 | 2.39 | Protein, |
| | | | Phosphorylation |
| CAMK1 | Calcium/calmodulin-dependent protein kinase type 1 | 1.25 | Protein |
| CAMK1D | Calcium/calmodulin-dependent protein kinase type | 5.06 | Protein, |
| | 1D | | Phosphorylation |
| CAMK2G | Calcium/calmodulin-dependent protein kinase type | 2.45 | Protein, |
| | II subunit gamma | | Phosphorylation |
| CAMK4 | Calcium/calmodulin-dependent protein kinase type IV | 0.86 | Protein |
| CDC42BPB | Serine/threonine-protein kinase MRCK beta | 1.52 | Protein, |
| | | | Phosphorylation |
| CDK10 | Cyclin-dependent kinase 10 | 1.66 | Protein, |
| | | | Phosphorylation |
| CDKL5 | Cyclin-dependent kinase-like 5 | 1.32 | Protein |
| CLK4 | Dual specificity protein kinase CLK4 | 1.37 | Protein |
| DAPK3 | Death-associated protein kinase 3 | 2.40 | Protein |
| DDR1 | Epithelial discoidin domain-containing receptor 1 | 2.16 | Protein |
| EGFR | Epidermal growth factor receptor | 2.89 | Protein, |
| | | | Phosphorylation |
| EIF2AK2 | Interferon-induced, double-stranded RNA-activated | 1.09 | Protein |
| | protein kinase | | |
| EIF2AK4 | Eukaryotic translation initiation factor 2-alpha | 1.46 | Protein |
| | kinase 4 | | |
| EPHA2 | Ephrin type-A receptor 2 | 1.15 | Protein, |
| | | | Phosphorylation |
| EPHA3 | Ephrin type-A receptor 3 | 1.32 | Phosphorylation |
| EPHA4 | Ephrin type-A receptor 4 | 2.16 | Protein, |
| | | | Phosphorylation |
| EPHA5 | Ephrin type-A receptor 5 | 1.32 | Phosphorylation |
| EPHB1 | Ephrin type-B receptor 1 | 3.10 | Protein, |
| | | | Phosphorylation |
| EPHB2 | Ephrin type-B receptor 2 | 7.14 | Protein, |
| | | | Phosphorylation |
| EPHB6 | Ephrin type-B receptor 6 | 2.49 | Protein |
| FER | Tyrosine-protein kinase Fer | 1.61 | Phosphorylation |
| FGFR1 | Fibroblast growth factor receptor 1 | 1.98 | Protein |
| FYN | Tyrosine-protein kinase Fyn | 3.46 | Protein, |
| | | | Phosphorylation |
| GSK3A | Glycogen synthase kinase-3 alpha | 0.60 | Protein, |
| | | | Phosphorylation |
| GSK3B | Glycogen synthase kinase-3 beta | 0.60 | Protein, |
| | | | Phosphorylation |
| HCK | Tyrosine-protein kinase HCK | 1.12 | Protein |
| HIPK2 | Homeodomain-interacting protein kinase 2 | 2.02 | Protein, |
| | | | Phosphorylation |
| HSPB8 | Heat shock protein beta-8 | 1.55 | Protein |
| JAK1 | Tyrosine-protein kinase JAK1 | 1.06 | Protein |
| JAK2 | Tyrosine-protein kinase JAK2 | 2.44 | Protein |
| LCK | Tyrosine-protein kinase Lc | 0.82 | Phosphorylation |
| LIMK1 | LIM domain kinase 1 | 1.76 | Protein, |
| | | | Phosphorylation |
| LYN | Tyrosine-protein kinase Lyn | 1.81 | Protein, |
| | | | Phosphorylation |
| MAP3K10 | Mitogen-activated protein kinase kinase kinase 10 | 0.79 | Phosphorylation |
| MAP3K12 | Mitogen-activated protein kinase kinase kinase 12 | 1.58 | Phosphorylation |
| MAP3K2 | Mitogen-activated protein kinase kinase kinase 2 | 1.81 | Protein, |
| | | | Phosphorylation |
| MAP3K3 | Mitogen-activated protein kinase kinase kinase 3 | 1.47 | Phosphorylation |
| MAP3K9 | Mitogen-activated protein kinase kinase kinase 9 | 1.83 | Phosphorylation |
| MAP4K5 | Mitogen-activated protein kinase kinase kinase | 1.83 | Protein, |
| | kinase 5 | | Phosphorylation |
| MAPK10 | Mitogen-activated protein kinase 10 | 1.06 | Phosphorylation |
| MAPK8 | Mitogen-activated protein kinase 8 | 1.06 | Phosphorylation |
| MAPK9 | Mitogen-activated protein kinase 9 | 1.01 | Phosphorylation |
| MARK4 | MAP/microtubule affinity-regulating kinase 4 | 1.15 | Protein |
| MET | Hepatocyte growth factor receptor | 4.21 | Protein, |
| | | | Phosphorylation |
| MINK1 | Misshapen-like kinase 1 | 1.28 | Protein, |
| | | | Phosphorylation |
| MYLK | Myosin light chain kinase, smooth muscle | 1.40 | Phosphorylation |
| NEK9 | Serine/threonine-protein kinase Nek9 | 0.43 | Protein |
| NRBP1 | Nuclear receptor-binding protein | 0.91 | Phosphorylation |
| NRK | Nik-related protein kinase | 1.22 | Phosphorylation |
| NUAK1 | NUAK family SNF1-like kinase 1 | 3.59 | Protein, |
| | | | Phosphorylation |
| NUAK2 | NUAK family SNF1-like kinase 2 | 2.28 | Protein, |
| | | | Phosphorylation |
| PAK4 | Serine/threonine-protein kinase PAK 4 | 2.41 | Protein |
| PKMYT1 | Membrane-associated tyrosine-and threonine- | 1.11 | Protein |
| | specific cdc2-inhibitory kinase | | |
| PKN1 | Serine/threonine-protein kinase N1 | 0.62 | Protein |
| PRKAA1 | 5-AMP-activated protein kinase catalytic subunit | 0.77 | Protein |
| | alpha-1 | | |
| PRKD3 | Serine/threonine-protein kinase D3 | 1.30 | Phosphorylation |
| PTK2 | Focal adhesion kinase 1 | 1.57 | Protein, |
| | | | Phosphorylation |
| RIPK2 | Receptor-interacting serine/threonine-protein kinase | 0.90 | Protein |
| | 2 | | |
| RPS6KA2 | Ribosomal protein S6 kinase; Ribosomal protein S6 | 1.21 | Protein |
| | kinase alpha-2 | | |
| RPS6KA4 | Ribosomal protein S6 kinase alpha-4; Ribosomal | 1.29 | Protein |
| | protein S6 kinase | | |
| RPS6KA5 | Ribosomal protein S6 kinase alpha-5 | 2.54 | Protein |
| SIK1 | Serine/threonine-protein kinase SIK1 | 2.21 | Protein, |
| | | | Phosphorylation |
| SRC | Proto-oncogene tyrosine-protein kinase Src | 0.99 | Phosphorylation |
| STK10 | Serine/threonine-protein kinase 10 | 1.28 | Protein, |
| | | | Phosphorylation |
| STK17A | Serine/threonine-protein kinase 17A | 2.68 | Protein, |
| | | | Phosphorylation |
| STK17B | Serine/threonine-protein kinase 17B | 2.16 | Protein |
| STK25 | Serine/threonine-protein kinase 25 | 1.86 | Protein |
| STK3 | Serine/threonine-protein kinase 32B | 0.88 | Phosphorylation |
| STK32B | Serine/threonine-protein kinase 32B | 2.73 | Protein |
| STK4 | Serine/threonine-protein kinase 4 | 0.88 | Phosphorylation |
| STRADA | STE20-related kinase adapter protein alpha | 0.94 | Protein |
| TBK1 | Serine/threonine-protein kinase TBK1 | 0.57 | Protein |
| TEC | Tyrosine-protein kinase Tec | 2.59 | Protein |
| TNIK | TRAF2 and NCK-interacting protein kinase | 2.01 | Phosphorylation |
| TNK2 | Activated CDC42 kinase 1 | 1.83 | Protein, |
| | | | Phosphorylation |
| TTK | Dual specificity protein kinase TTK | 1.62 | Protein |
| ULK1 | Serine/threonine-protein kinase ULK1 | 1.14 | Phosphorylation |
| ULK2 | Serine/threonine-protein kinase ULK2 | 1.83 | Phosphorylation |
| YES1 | Tyrosine-protein kinase Yes | 0.82 | Phosphorylation |
| ZAK | Mitogen-activated protein kinase kinase kinase MLT | 2.58 | Protein, |
| | | | Phosphorylation |

In specific embodiments, the EMT-associated kinase targeted in the method is selected from AXL, MET, EPHB2, FYN, AKT3, CAMK1D, NUAK1, NUAK2, EPHA4, CAMK1D, FYN, NEK3, CDK3, PLK1, CHEK1, EGFR, HIPK2, TNK2, LYN, PTK2, MAP3K12, MAPK9, MAPK8, FER, AAK1, CDK10, STK17B, and STK32B.

Any agent that targets an EMT-associated kinase for inhibition of expression or prevention of activity (e.g., via modulation of phosphorylation, or inhibition of critical interacting proteins, or interrupting other aspects of the signaling pathway associated with the EMT-associated kinase) is encompassed by the present disclosure. In some embodiments, the agent reduces the functional expression of the target EMT-associated kinase. For example, RNA interference (RNAi) technologies, including siRNA or shRNA approaches, can be readily implemented to specifically knockdown functional expression of desired target EMT-associated kinases based on the known sequence of the encoding mRNA of the kinases (e.g., kinases identified in Table 2). Additionally, small molecule pharmaceuticals and antibody-based therapeutics that inhibit the kinases identified in Table 2 are known and are encompassed by the present disclosure. Exemplary, non-limiting agents that can be used to inhibit target EMT-associated kinases according to select embodiments of the disclosure are set forth in Table 3, although additional examples are known to persons of ordinary skill in the art.

TABLE 3
Exemplary agents targeting selected EMT-associated kinases.

| Target | | | |
|---|---|---|---|
| Kinase | Example Inhibitor | CAS No. | Other Targets |
| AAK1 | LP-935509 | 1454555-29-3 | BMP2K, GAK |
| ABL1 | Imatinib (STI571) | 152459-95-5 | PDGFR, KIT |
| ABL2 | Imatinib (STI571) | 152459-95-5 | PDGFR, KIT |
| ACVR1 | LDN-193189 | 1062368-24-4 | BMPR1A |
| AKT1 | MK-2206 2HCl | 1032350-13-2 | AKT2, AKT3 |
| AKT3 | MK-2206 2HCl | 1032350-13-2 | AKT1, AKT2 |
| AXL | Cabozantinib (BMS-907351) | 849217-68-1 | VEGFR2, MET, RET, KIT |
| BMP2K | LP-935509 | 1454555-29-3 | AAK1, GAK |
| CAMK1D | Compound 19 (Fromont et al., J. | | |
| | Med. Chem., 2020) | | |
| CLK4 | ML167 | 1285702-20-6 | CLK1, CLK2, CLK3 |
| DDR1 | Dasatinib (BMS-354825) | 302962-49-8 | SRC, Ephrin Receptors |
| EGFR | Lapatinib | 231277-92-2 | |
| EPHA2 | Dasatinib (BMS-354825) | 302962-49-8 | SRC, Ephrin Receptors |
| EPHA3 | Dasatinib (BMS-354825) | 302962-49-8 | SRC, Ephrin Receptors |
| EPHA4 | Dasatinib (BMS-354825) | 302962-49-8 | SRC, Ephrin Receptors |
| EPHA5 | Dasatinib (BMS-354825) | 302962-49-8 | SRC, Ephrin Receptors |
| EPHB1 | Dasatinib (BMS-354825) | 302962-49-8 | SRC, Ephrin Receptors |
| EPHB2 | Dasatinib (BMS-354825) | 302962-49-8 | SRC, Ephrin Receptors |
| EPHB6 | Dasatinib (BMS-354825) | 302962-49-8 | SRC, Ephrin Receptors |
| FER | Dasatinib (BMS-354825) | 302962-49-8 | SRC, Ephrin Receptors |
| FGFR1 | Pazopanib HCl | 635702-64-6 | FGFR2, FGFR3, FGFR4, |
| | (GW786034 HCl) | | VEGFR, PDGFR |
| FYN | Dasatinib (BMS-354825) | 302962-49-8 | SRC, Ephrin Receptors |
| GSK3A | CHIR-99021 (CT99021) HCl | 1797989-42-4 | GSK3B |
| GSK3B | CHIR-99021 (CT99021) HCl | 1797989-42-4 | GSK3A |
| JAK1 | Ruxolitinib (INCB018424) | 941678-49-5 | JAK2 |
| JAK2 | Ruxolitinib (INCB018424) | 941678-49-5 | JAK1 |
| LCK | Dasatinib (BMS-354825) | 302962-49-8 | SRC, Ephrin Receptors |
| LYN | Dasatinib (BMS-354825) | 302962-49-8 | SRC, Ephrin Receptors |
| MAPK10 | JNK-IN-8 | 1410880-22-6 | MAPK8, MAPK9, MAPK10 |
| MAPK8 | JNK-IN-8 | 1410880-22-6 | MAPK8, MAPK9, MAPK10 |
| MAPK9 | JNK-IN-8 | 1410880-22-6 | MAPK8, MAPK9, MAPK10 |
| MET | Cabozantinib (BMS-907351) | 849217-68-1 | VEGFR2, MET, RET, KIT |
| NUAK1 | WZ4003 | 1214265-58-3 | NUAK2 |
| NUAK2 | WZ4003 | 1214265-58-3 | NUAK1 |
| PAK4 | KPT 9274 ( ATG-019) | 1643913-93-2 | NAMPT |
| PRKAA1 | Dorsomorphin (Compound C) | 1219168-18-9 | |
| | 2HCl | | |
| PTK2 | PF-431396 | 717906-29-1 | PTK2B |
| RIPK2 | GSK2983559 (compound 3) | 1579965-12-0 | |
| RPS6KA2 | S6K-18 | 1265789-88-5 | other S6Ks |
| RPS6KA4 | S6K-18 | 1265789-88-5 | other S6Ks |
| RPS6KA5 | S6K-18 | 1265789-88-5 | other S6Ks |
| SRC | Dasatinib (BMS-354825) | 302962-49-8 | SRC, Ephrin Receptors |
| STK3 | XMU-MP-1 | 2061980-01-4 | STK3, STK4 |
| STK4 | XMU-MP-1 | 2061980-01-4 | STK3, STK4 |
| TBK1 | TBK1/IKK_{ε}-IN-2 | 1292310-49-6 | |
| TNK2 | XMD16-5 | 1345098-78-3 | |
| ULK1 | MRT68921 HCl | 1190379-70-4 | ULK1, ULK2 |
| ULK2 | MRT68921 HCl | 1190379-70-4 | ULK1, ULK2 |
| YES1 | Dasatinib (BMS-354825) | 302962-49-8 | SRC, Ephrin Receptors |

The disclosed method can be incorporated into a combination method, wherein the method further comprises contacting the cell with the chemotherapeutic agent.

The method can be in vitro, e.g., in cell culture, to assess susceptibility of the cancer cell to the combination treatment or study underlying kinome signaling, and the like. In some embodiments, the cell is contacted in vivo in a subject with cancer. Such embodiments can be methods of treatment and comprise administering a therapeutically effective amount of the agent that inhibits the expression or function of the EMT-associated kinase. In further embodiments, the method further comprises also administering to the subject the chemotherapeutic agent. The agent and chemotherapeutic agent can be administered together or in a coordinated fashion, e.g., with the agent that inhibits the expression or function of an epithelial-mesenchymal transition (EMT)-associated kinase being administered before or after the chemotherapeutic agent. The patient can receive a dosing regimen of several coordinated administrations of the agent and the chemotherapeutic agent.

Accordingly, in another aspect, the disclosure provides aspect the disclosure provides a method of treating cancer in a subject. The method can be characterized as a method of enhancing sensitivity of a cancer cell to a kinase inhibitor therapy in a subject in need thereof, comprising administering to the subject an effective amount of an agent that inhibits the expression or function of an epithelial-mesenchymal transition (EMT)-associated kinase.

As used herein, the term “treat” refers to medical management of a disease, disorder, or condition (e.g., cancer, such as HCC, as described above) of a subject (e.g., a human or non-human mammal, such as another primate, horse, dog, mouse, rat, guinea pig, rabbit, and the like). Treatment can encompasses any indicia of success in the treatment or amelioration of a disease or condition (e.g., a cancer, such as HCC), including any parameter such as abatement, remission, diminishing of symptoms or making the disease or condition more tolerable to the patient, slowing in the rate of degeneration or decline, or making the degeneration less debilitating. Specifically in the context of cancer, the term treat can encompass slowing or inhibiting the rate of cancer growth, or reducing the likelihood of recurrence, compared to not having the treatment. In some embodiments, the treatment encompasses resulting in some detectable degree of cancer cell death in the patient. The treatment or amelioration of symptoms can be based on objective or subjective parameters, including the results of an examination by a physician. Accordingly, the term “treating” includes the administration of the compositions disclosed in the present disclosure to alleviate, or to arrest or inhibit development of the symptoms or conditions associated with disease or condition (e.g., cancer). The term “therapeutic effect” refers to the amelioration, reduction, or elimination of the disease or condition, symptoms of the disease or condition, or side effects of the disease or condition in the subject. The term “therapeutically effective” refers to an amount of the composition that results in a therapeutic effect and can be readily determined. In some embodiments, the disclosed agent prevents or reverses the EMT of the cancer cells in the subject, allowing for additional treatment by chemotherapy, such as chemotherapy targeting kinases (e.g., kinase inhibitor-based therapy).

Exemplary kinase inhibitor therapy targets, kinase inhibitor therapies, and agents that inhibit the expression or function of an epithelial-mesenchymal transition (EMT)-associated kinase are described above and are encompassed in this aspect.

In a specific embodiment, the subject has hepatocellular carcinoma (HCC) and the cancer cell is an HCC cell.

In some embodiments, the method is incorporated into a combination therapy whereby the method further comprises administering to the subject a therapeutically effective amount of the chemotherapeutic agent to treat the subject.

In another aspect, the disclosure also provides formulations and kits comprising the formulations appropriate for methods of administration for application to in vivo therapeutic settings in subjects (e.g., mammalian subjects with cancer). According to skill and knowledge common in the art, the disclosed agent and chemotherapeutic agents can be formulated with appropriate carriers and non-active binders, and the like, for administration to target specific tumor and/or cancer cells

In another aspect, the disclosure provides a method of profiling the kinome in a cell or population of similar cells. The method comprises:

contacting a cell lysate with a population kinase capture reagents, wherein the kinase capture reagents comprise one or more of kinase target moieties linked to a bead;

isolating from the lysate kinases or complexes comprising a kinase that are bound by the kinase capture reagents;

digesting the kinases and any associated proteins in a complex comprising a kinase to provide a peptide sample;

conducting liquid chromatography-mass spectroscopy (LC-MS) on the peptide sample;

identifying the presence or abundance of one or more kinases and/or one or more kinase interacting proteins, or phosphorylation state thereof, based on results of the LC-MS.

The one or more kinase target moieties can comprise ATP, ATP analogs, and/or kinase inhibitors.

The population of similar cells can be from the same tissue sample, e.g., derived from the same originating tissue in a subject or from the same biological sample obtained from the subject.

In some embodiments, the method comprises identifying complexes of kinases and kinase interacting proteins by LC-MS.

In some embodiments, the method further comprises correlating the presence or abundance of one or more kinases and/or one or more kinase interacting proteins, or the phosphorylation state thereof, with a drug response, cell phenotype, or cell marker expression. This correlation can be made by profiling the kinomes of cells representing, e.g., the difference drug responses, phenotypes, cell marker expression profiles, etc., and comparing the resulting kinome profiles.

### General Definitions

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Current Protocols in Immunology, John Wiley & Sons, New York (2010), Mirzaei, H. and Carrasco, M. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed herein, in the specification, examples and appended claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, which is to indicate, in the sense of “including, but not limited to.” Words using the singular or plural number also include the plural and singular number, respectively. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:

(1) Alanine (A), Serine (S), Threonine (T),

(2) Aspartic acid (D), Glutamic acid (E),

(3) Asparagine (N), Glutamine (Q),

(4) Arginine (R), Lysine (K),

(5) Isoleucine (I), Leucine (L), Methionine (M), Valine (V), and

(6) Phenylalanine (F), Tyrosine (Y), Tryptophan (W).

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

The following describes the initial design and implementation of a pharmacoproteomic assay platform to identify kinome features that underlie drug response in hepatocellular carcinoma (HCC). Select targets identified from the pipeline were validated, demonstrating the robustness of the approach to identify viable therapeutic targets to enhance therapeutic interventions for HCC. The study was published in Golkowski, M., et al. (2020) Pharmacoproteomics Identifies Kinase Pathways that Drive the Epithelial-Mesenchymal Transition and Drug Resistance in Hepatocellular Carcinoma. Cell Systems. 11(2):196-207.e7, incorporated herein by reference in its entirety.

### Abstract

Hepatocellular carcinoma (HCC) is a complex and deadly disease. Lacking in genetic mutations that can be targeted by molecular therapies, the few available treatment options for advanced HCC have limited efficacy. To improve responses to existing HCC drugs, predictive biomarkers are urgently needed to select appropriate therapies for individual HCC patients. Most HCC drugs target protein kinases, highlighting that kinase-dependent signaling networks drive HCC progression. The inventors investigated HCC-specific kinase activities to identify potential markers of drug response and novel drug targets. This included development of an unbiased pharmacoproteomics approach to identify signaling networks that determine HCC responses to kinase inhibitors (KIs). In a panel of 17 HCC cell lines, kinome activity was quantified with kinobead/LC-MS profiling and these cells were screened against 299 KIs to measure growth inhibition. Integrating kinome activity with KI responses using gene set enrichment analysis (GSEA), a comprehensive dataset of pathway-based drug response signatures was created, and by profiling patient HCC samples with kinobeads signatures of clinical HCC drug responses enriched in individual tumors were identified. Furthermore, these analyses identified signaling networks promoting the HCC cell epithelial-mesenchymal transition (EMT) and drug resistance, including a novel FZD2-AXL-NUAK1/2 signaling module that, when modulated by genetic or small-molecule inhibition, reversed the EMT and sensitized HCC cells to drugs. This kinome-centric pharmacoproteomics approach conclusively identifies novel cancer drug targets, and molecular signatures of drug response for personalized oncology.

### Results

Kinome-Centric Pharmacoproteomics Analysis of HCC

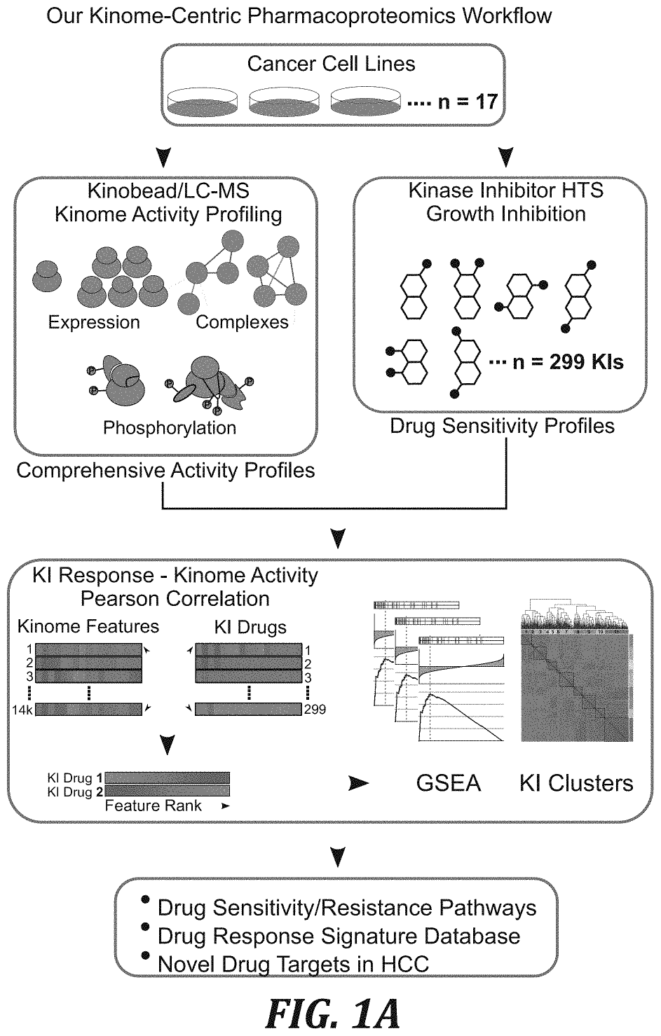

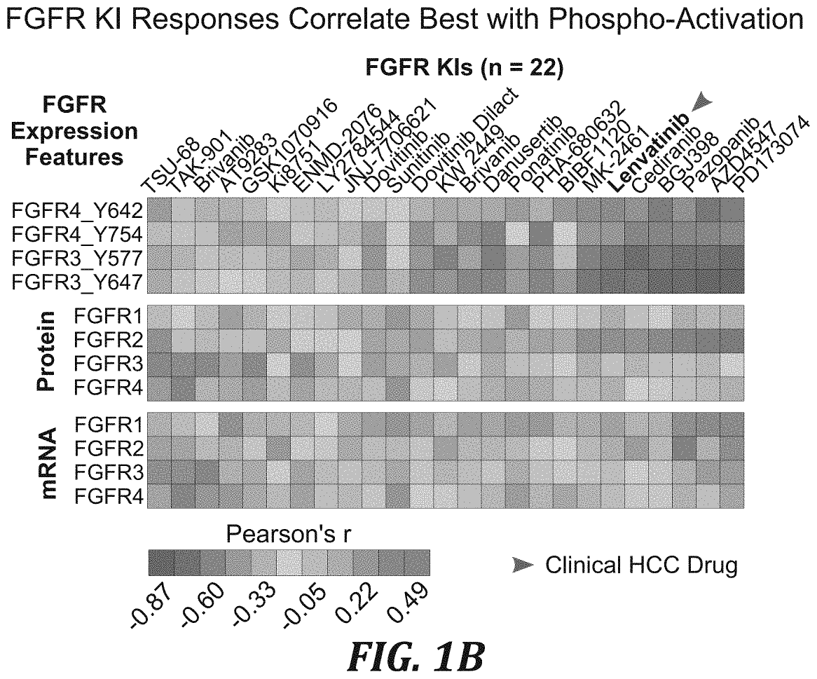

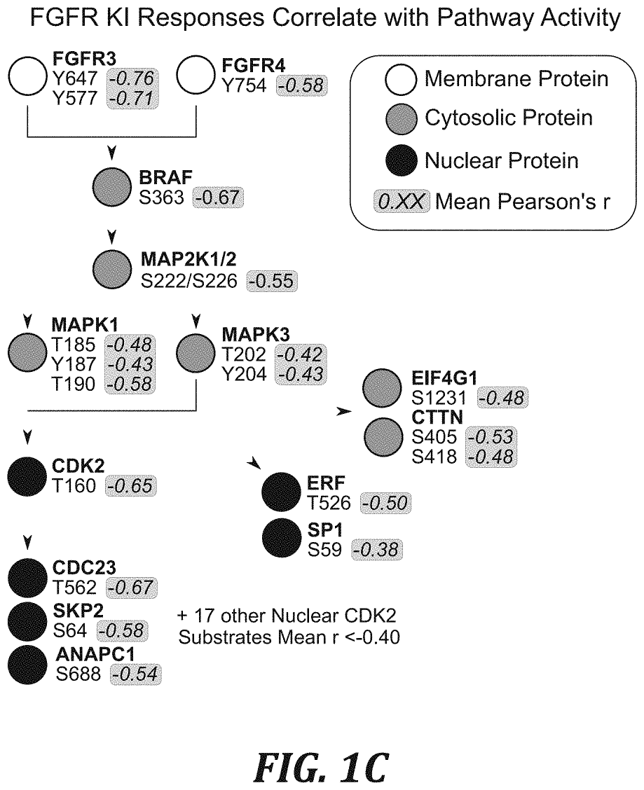

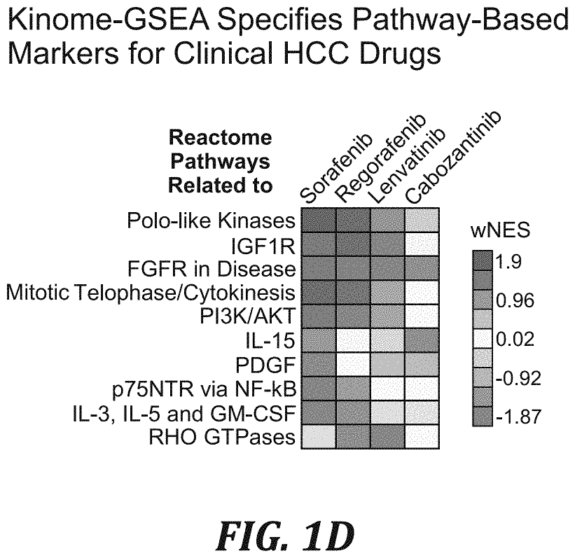

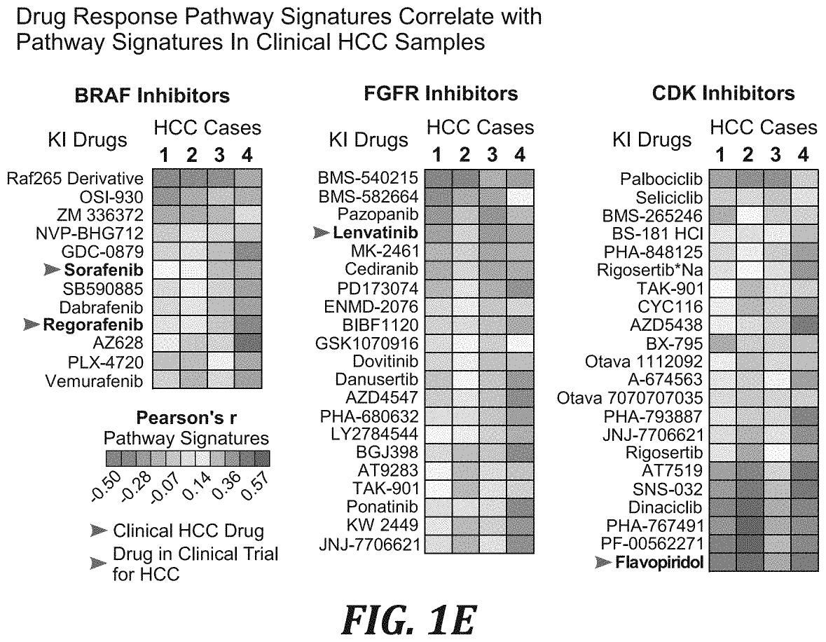

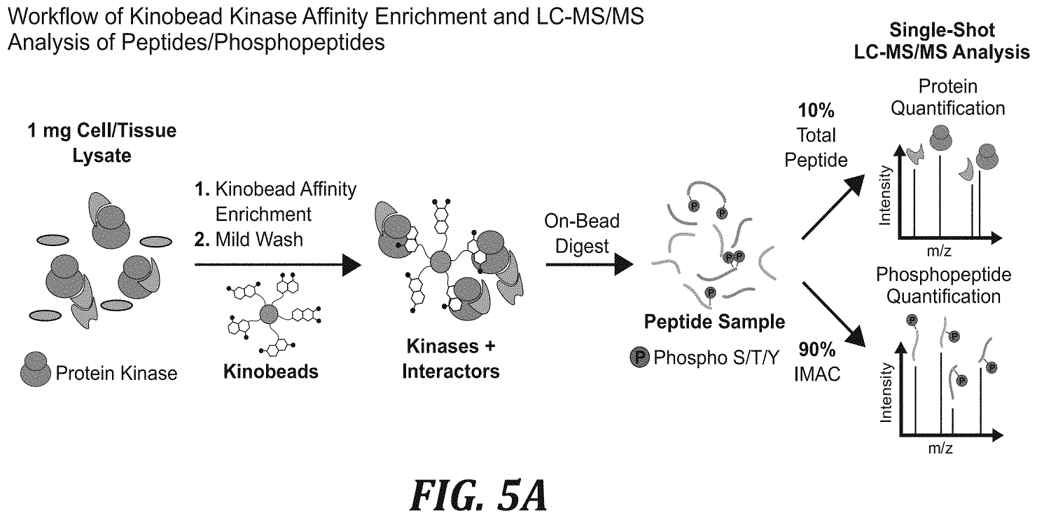

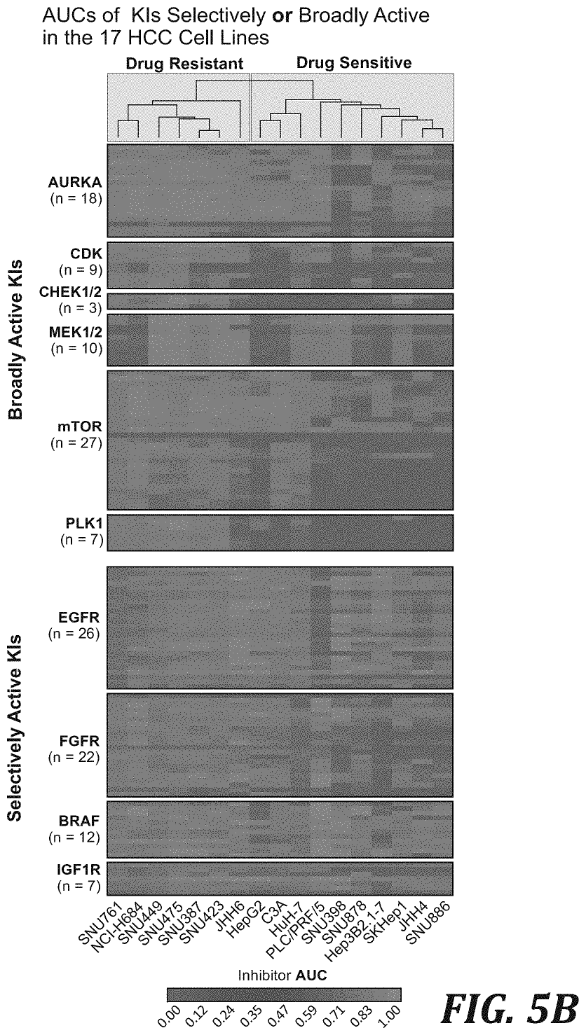

A panel of 17 HCC lines was selected from the Cancer Cell Line Encyclopedia (CCLE) (Barretina, J., et al. (2012). The Cancer Cell Line Encyclopedia enables predictive modelling of anticancer drug sensitivity. Nature 483, 603-607, incorporated herein by reference in its entirety) with diverse kinase mRNA expression and profiled these using the kinobead/LC-MS platform described herein to measure kinase expression, their phosphorylation states, and kinase-interacting proteins (FIGS. 1A and 5A) (Golkowski, M., et al. (2020). Kinobead/LC-MS Phosphokinome Profiling Enables Rapid Analyses of Kinase-Dependent Cell Signaling Networks. Journal of Proteome Research 19, 1235-1247, incorporated herein by reference in its entirety). This approach quantified 2731 proteins and 11204 phosphorylation sites, including 346 kinases, 2821 kinase phosphorylation sites, and 886 kinase-interacting proteins. Functionally characterized phosphosites (Hornbeck, P. V., et al. (2015). PhosphoSitePlus, 2014: mutations, PTMs and recalibrations. Nucleic Acids Research 43, D512-520., incorporated herein by reference in its entirety), kinase-interacting proteins, and their phosphorylation sites specified the activation states of 284 of the 346 kinases (see ‘STAR methods’). A 299-member diversity library of experimental, preclinical and clinical KIs that together inhibit at least 145 primary kinase targets (see Table 1) was used to obtain seven-point dose-response curves for each KI in all 17 HCC cell lines (see ‘STAR Methods’, FIG. 1A). Using the area under the dose-response curve (AUC) as a measure of drug efficacy, diverse responses to inhibitors were observed. For instance, FGFR, EGFR, IGF1R and BRAF inhibitors, blocked cell growth in certain HCC lines, while inhibitors of MEK, cell cycle-related kinases and MTOR were broadly active, efficiently inhibiting cell growth in 10 of the 17 cell lines (FIG. 5B). The two resulting datasets thus provide deep proteomic coverage of kinome activity and diverse cellular response to KI drugs across the 17 cell line panel.

Kinase Activation States are Powerful Predictors of Drug Response

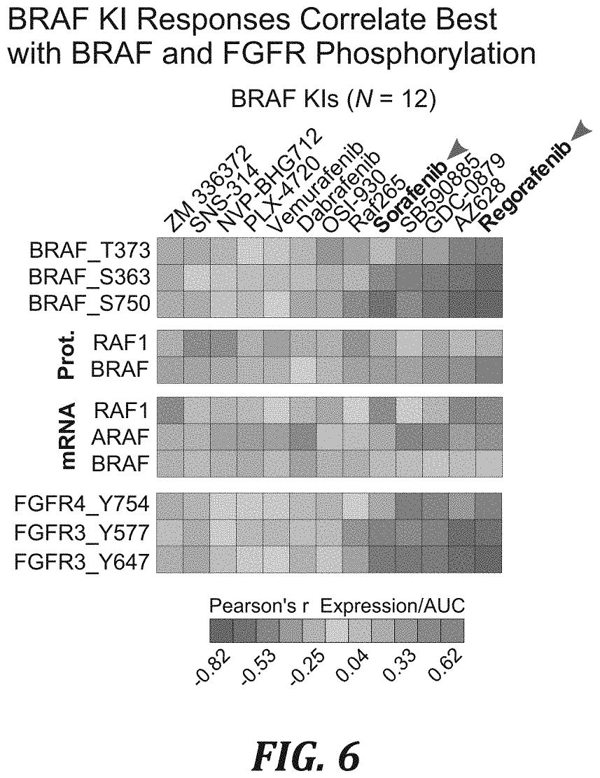

To rank proteomics features by their association with drug responses, the MS intensities of all 13935 quantified features were correlated with each KI's AUC values across the 17 HCC line panel, where r<0 indicates drug sensitivity (high MS intensity—low AUC) while r>0 denotes cell survival and KI resistance (high MS intensity—high AUC, see ‘STAR Methods’). For example, the correlation of FGFR expression features with the response to 22 FGFR inhibitors, including lenvatinib was examined (FIG. 1B). Activating FGFR3 phosphorylation sites Y647 and Y577, and the activating Y754 on FGFR4 correlated very well with responses to most FGFR inhibitors (mean r=−0.40), whereas kinase protein and mRNA expression correlated much less (mean r=−0.12 and −0.09, FIG. 1B). Similarly, sensitivity to 12 BRAF inhibitors including sorafenib and regorafenib correlated better with kinase phosphorylation and activation than mRNA or protein expression (FIG. 6A). Analyzing the correlation of other phosphosites, it was observed that sites on numerous FGFR pathway members were tightly linked with FGFR inhibitor sensitivity (FIG. 1C). For instance, activating sites on MEK1/2, ERK1/2 and CDK2 (T160), and substrates of these kinases that regulate the cell cycle (e.g. CDCl₂3 and SKP2), transcription (ERF and SP1) and translation (EIF4G1) all correlated with FGFR KI responses (FIG. 1C). Together, these results show that: 1) the kinome-centric pharmacoproteomics approach correctly links proteomics features with drug response; 2) the activation state of kinases is often a better predictor of drug response than protein and mRNA expression; and that 3) phosphorylation events spanning the broader signaling network can predict the response to drugs that target other kinases within these pathways. The last point is of high interest because the unbiased identification of pathway-based drug response signatures could reveal novel mechanisms of disease and new druggable targets.

Unbiased GSEA of Kinome Features Defines Pathway-Based Drug Response Signatures

To identify pathway-based drug response signatures using a quantitative and statistical framework, GSEA was applied with 327 cancer-relevant Reactome pathways as the gene sets (Fabregat, A., et al. (2018). The Reactome Pathway Knowledgebase. Nucleic acids research 46, D649-D655; Kim, S., et al. (2012). Pathway-based classification of cancer subtypes. Biol Direct 7, 21; and Subramanian, A., et al. (2005). Gene set enrichment analysis: a knowledge-based approach for interpreting genome-wide expression profiles. Proc Natl Acad Sci USA 102, 15545-15550, each of which is incorporated herein by reference in its entirety) (see ‘STAR Methods’). This yielded normalized enrichment scores (NES) for 275 of the 327 pathways, ranking them for their association with sensitivity (positive NES) or resistance (negative NES) to each drug. Here, the selective FGFR and EGFR inhibitors, pazopanib and lapatinib, enriched pathways associated with FGFR and cell cycle activation or pathways associated with EGFR, PI3K and NF-κB activation (not shown). Remarkably, this analysis also identified pathways known to promote resistance to FGFR and EGFR inhibitors (i.e. with a negative NES) such as interleukin signaling (JAK-STAT pathway) and Wnt-signaling (not shown). While this analysis highlights the power of unbiased GSEA to identify pathways related to well characterized FGFR and EGFR inhibitors, the dataset should similarly identify signaling pathways related to the activity of other less well-studied KIs.

Analyzing drug response pathways of the clinical HCC drugs sorafenib, regorafenib and lenvatinib confirmed that these drugs are effective when FGFR and cell cycle pathways are active, and ineffective when survival pathways such as interleukin and NF-κB signaling are engaged (FIG. 1D). In contrast, the clinical AXL and MET inhibitor, cabozantinib, correlated less well with cell cycle pathway activity. To evaluate if the pathway-based drug response signatures can be detected in clinical HCC specimens, four tumor-normal adjacent liver (NAL) pairs with kinobead/LC-MS were analyzed. Gratifyingly, performance of the kinobead protocol in HCC tissue was comparable to the HCC cell line experiments and 2151 kinase phosphosites were quantified on 286 kinases, as well as 680 kinase interactors (not shown). GSEA was applied to identify Reactome pathways upregulated in tumors over NAL, correlating their pathway NES' with those from the 17 HCC cell lines to identify drug response signatures for each tumor sample (see ‘STAR Methods’). Strikingly, pathway-based signatures of clinical HCC drugs were highly enriched in specific tumors (FIG. 1E). For instance, HCC case 4 showed enrichment of pathways that specify sorafenib and regorafenib sensitivity (r of ˜0.4), as well as sensitivity to FGFR inhibitors. In contrast, CDK inhibitor pathway markers were highly enriched in three out of four tumors (r of ˜0.4 to ˜0.5, FIG. 1E), including flavopiridol that is currently in clinical trials in HCC. Collectively, these results suggest that kinome-centric pharmacoproteomics can identify drug response pathways in individual human tumor specimens and may inform selection of targeted therapies.

The HCC Cell EMT State Broadly Impacts Responses to Kinase Inhibitors

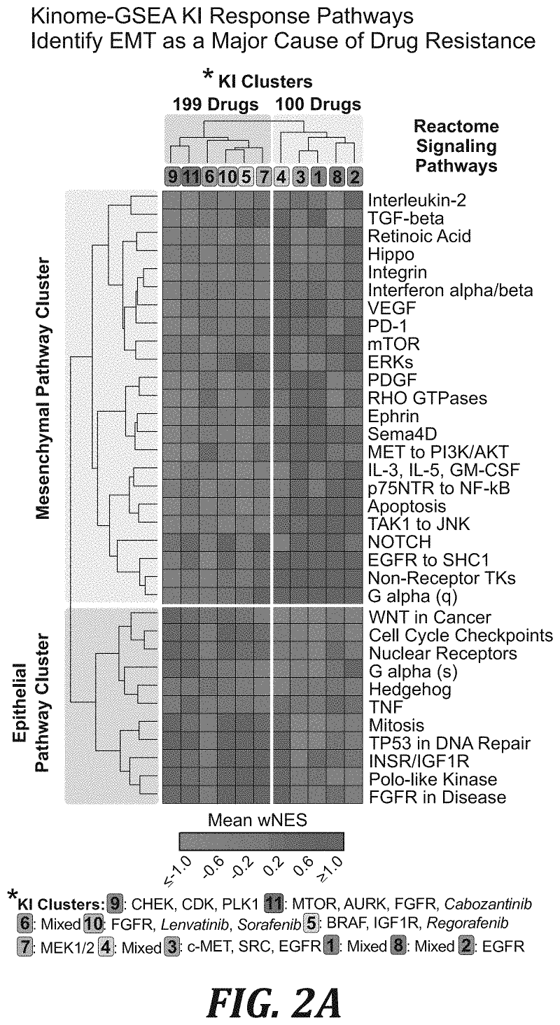

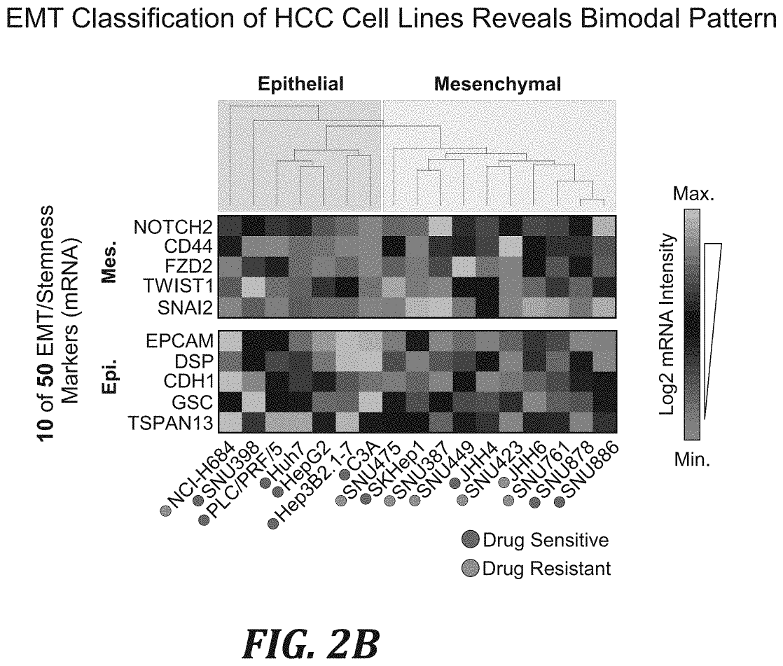

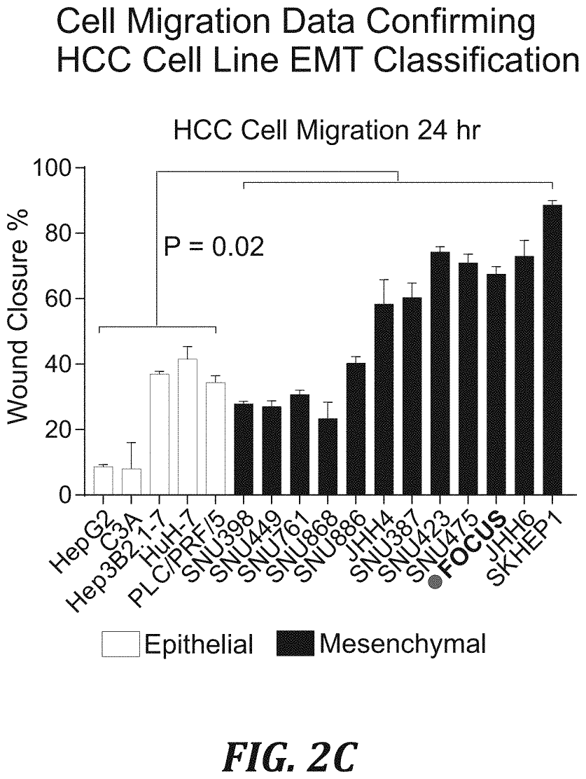

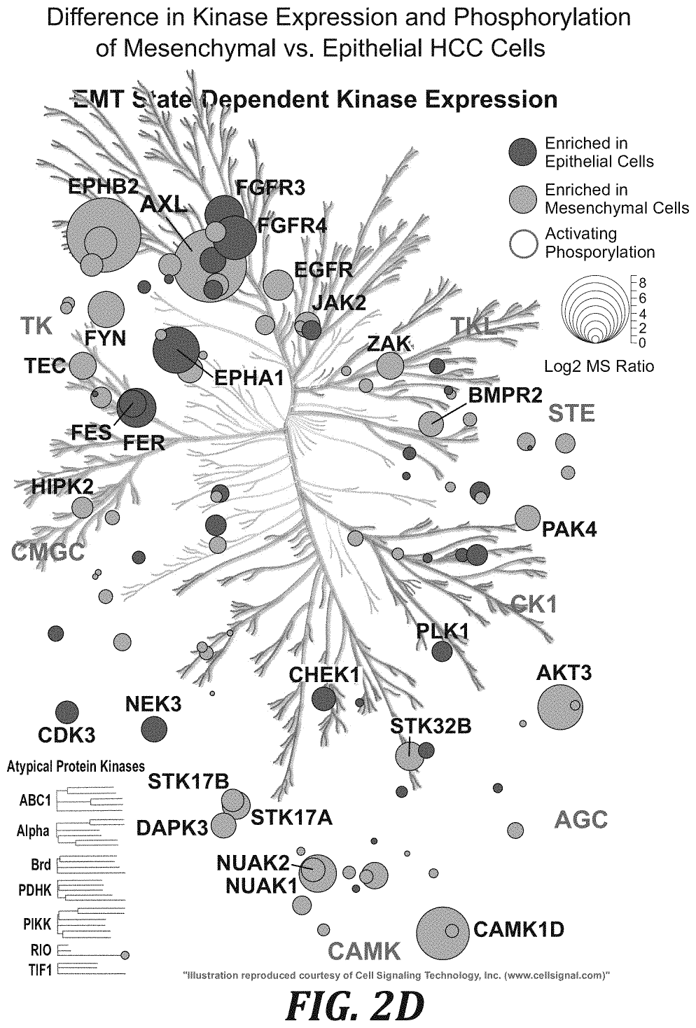

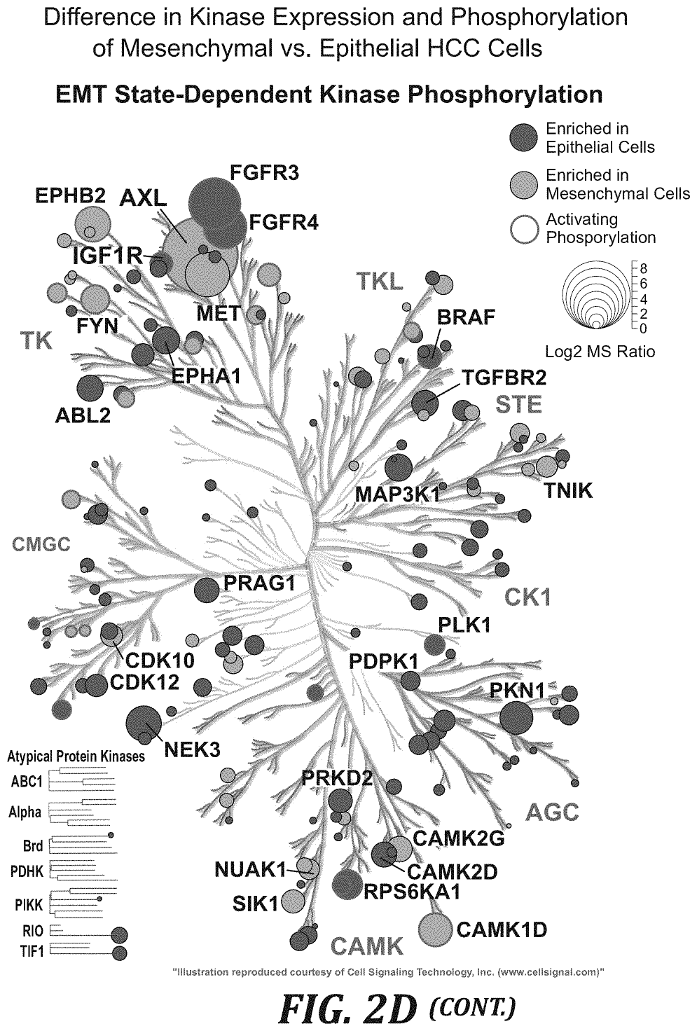

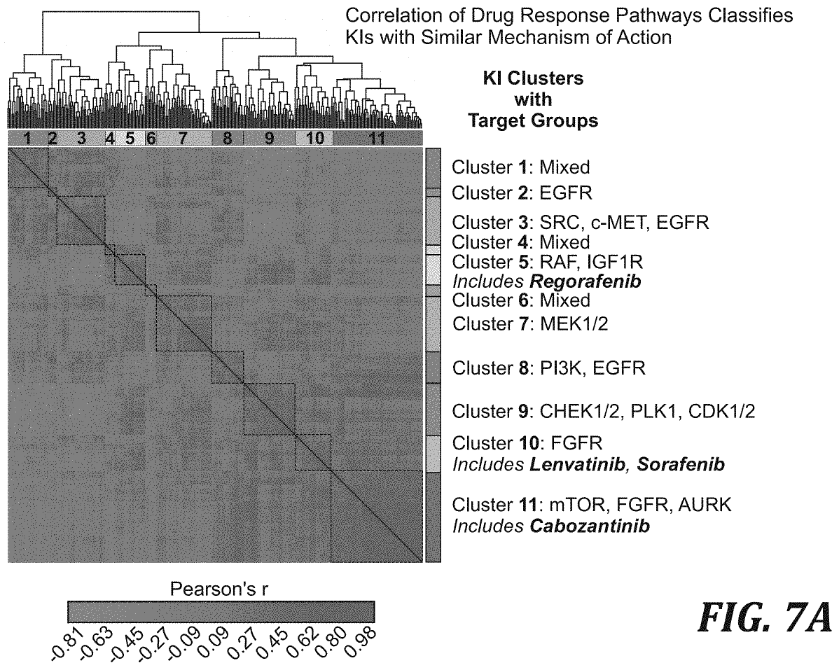

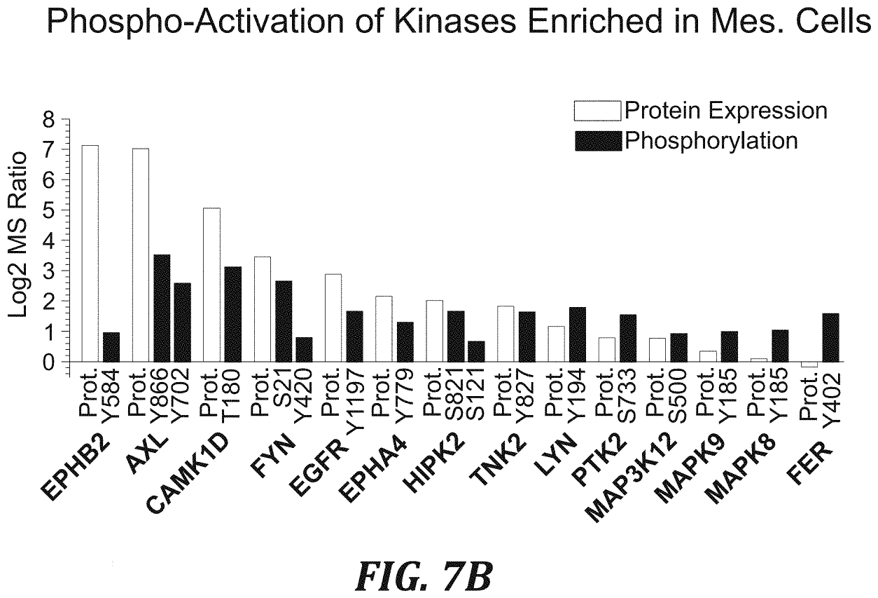

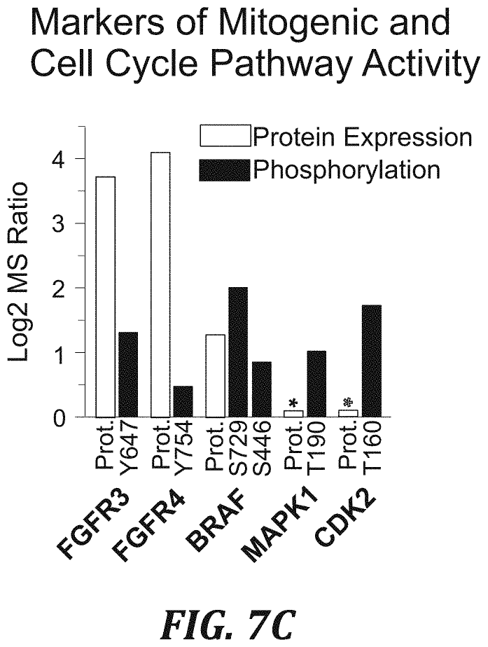

To identify the principal pathways that control responses to a broad range of clinical and pre-clinical KI drugs, similarities in response pathways among all 299 drugs tested (see Table 1) were explored in more detail. Drugs were classified into 11 KI clusters with similar pathway signatures and calculated mean NES values for 34 representative Reactome terms from the larger panel of 275 scored pathways, followed by unsupervised hierarchical clustering (see ‘STAR Methods’, FIGS. 2A and 7A). Strikingly, clustering produced a clear separation into two distinct groups. KIs in clusters 5-7 and 9-11 formed one group with positive NES values for pathways commonly overexpressed in rapidly proliferating cells, including FGFR-, IGF1R-, cell cycle-, and mitosis-related pathways. This group contained 199 drugs, mainly inhibiting BRAF, FGFR isoforms, the IGF1R, and cell cycle-related kinases (PLK1, CDKs, CHEK1/2), and had negative enrichment scores for pathways related to MET, TGF-β, cytokine and NF-kB signaling (FIG. 2A)—pathways that are known to regulate the EMT, an important mechanism of cancer cell metastasis and drug resistance. Conversely, 100 compounds in clusters 1-4 and 8 (e.g. EGFR, MET and SRC KIs) showed positive enrichment scores for these same EMT-associated pathways and negative scores for FGFR- and cell cycle-related terms (FIG. 2A). This opposing behavior of EMT pathway activation and KI drug response suggested: 1) the presence of mesenchymal HCC cells in this panel, and that 2) the EMT promotes resistance to two-thirds of the tested KI drugs. To test if this panel contains cell lines in different EMT states, the mRNA expression of 50 important EMT and stem cell markers were examined in the 17 HCC cell lines (Barretina, J., et al. (2012). The Cancer Cell Line Encyclopedia enables predictive modelling of anticancer drug sensitivity. Nature 483, 603-607, incorporated herein by reference in its entirety). Indeed, semi-supervised hierarchical clustering of EMT markers classified the panel into seven epithelial and ten mesenchymal lines (FIG. 2B).

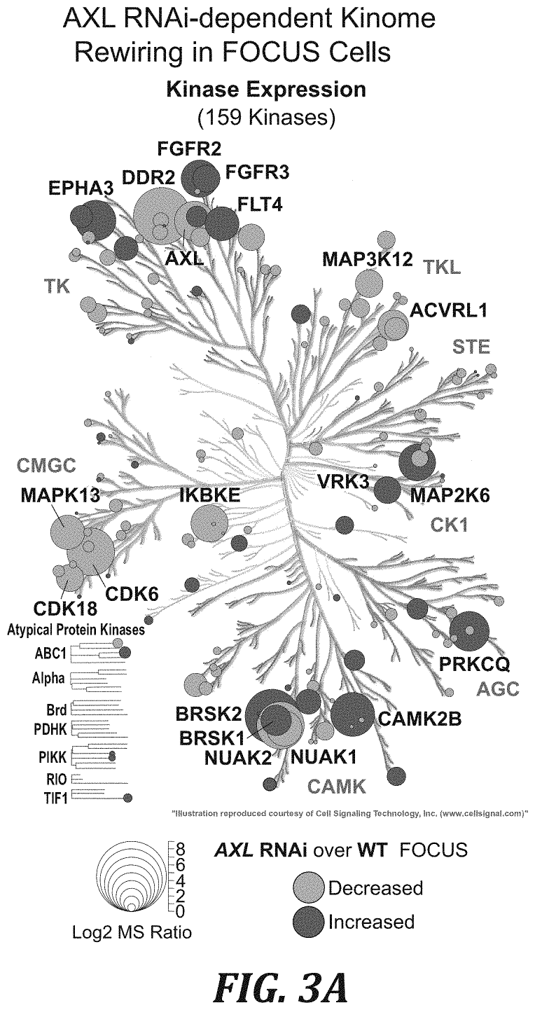

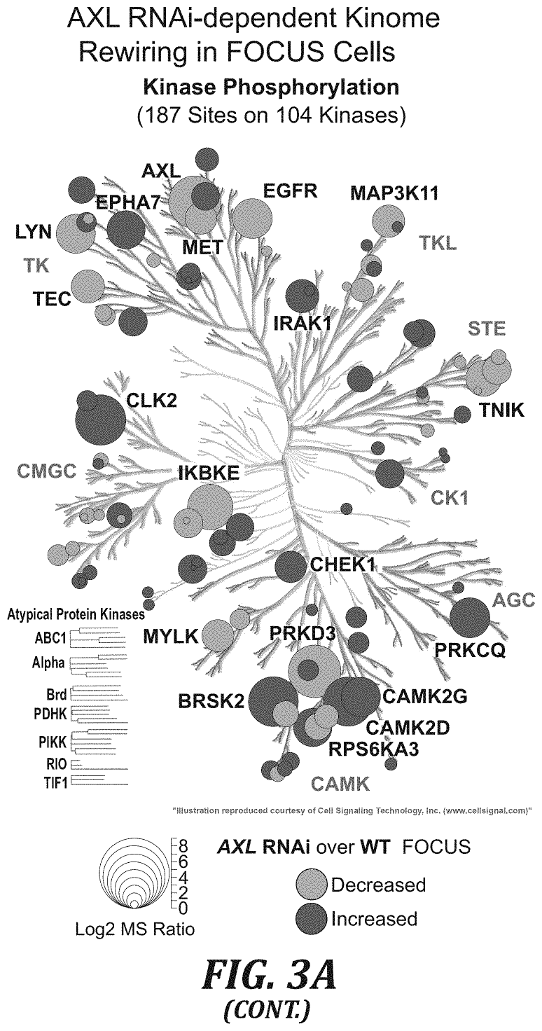

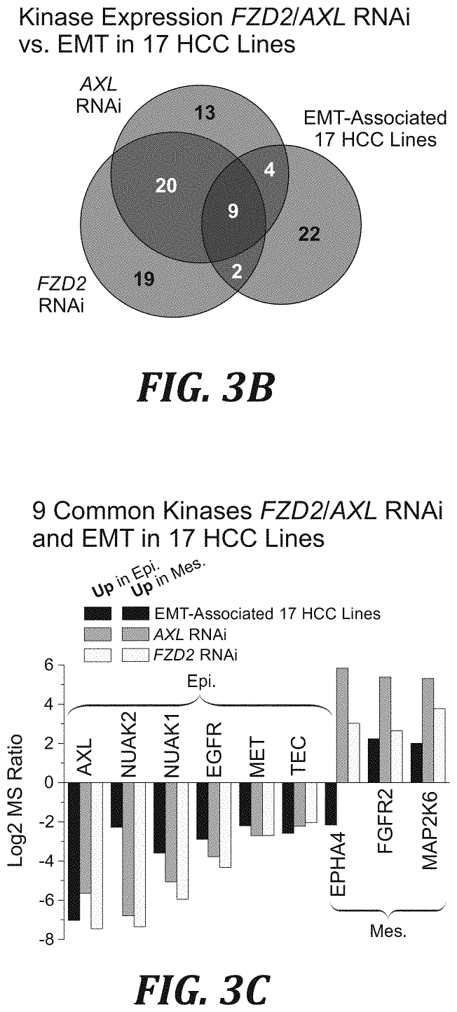

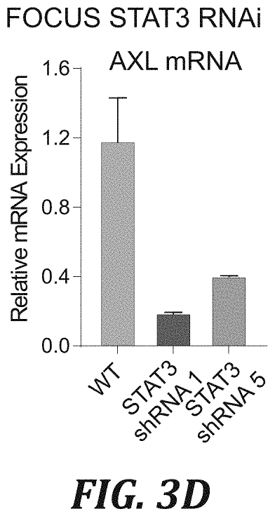

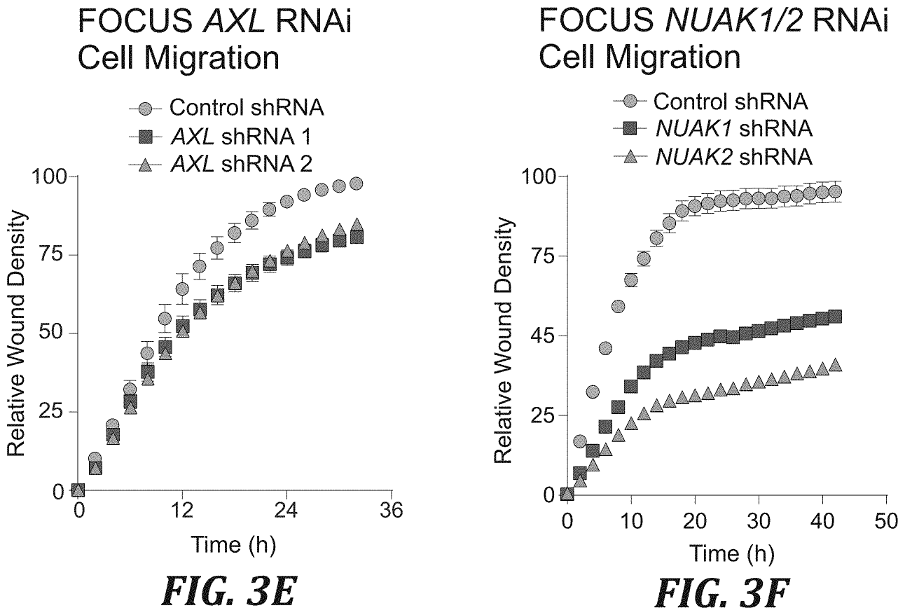

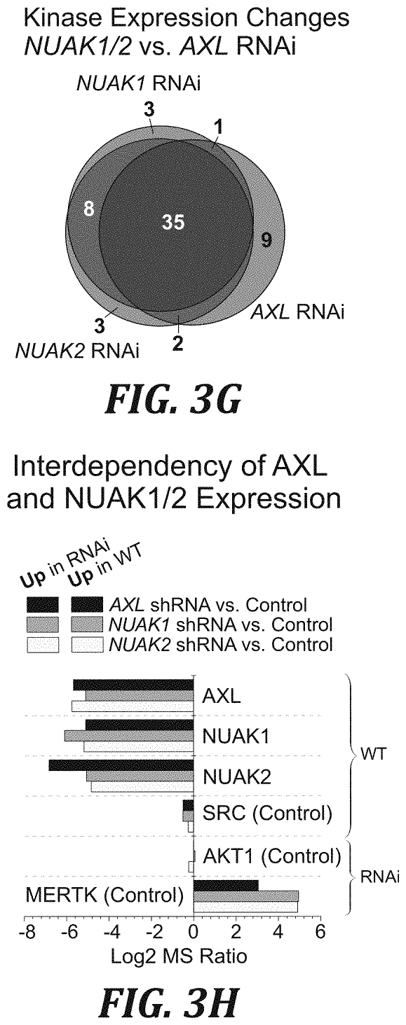

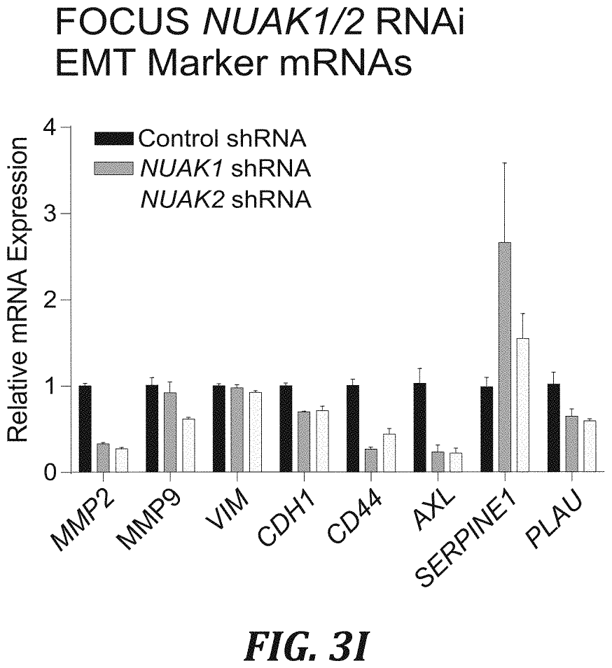

Because EMT is typically associated with increased cell motility, the cell migration of 16 of the 17 HCC lines as well as the mesenchymal FOCUS HCC line were also assayed, confirming that mesenchymal lines exhibited significantly enhanced wound closure compared to epithelial lines at 24 h (two sample T-test: P=0.02, FIG. 3C). Importantly, comparing the HCC line classification by drug response (FIG. 5B) with the classification by EMT marker expression (FIG. 2B), it was found that epithelial lines were highly enriched in the drug sensitive cluster (6 out of 7, hypergeometric T-test: P=0.0037). Collectively, these results suggest that the EMT state greatly impacts HCC cell responses to a broad range of KI drugs (e.g., the KIs listed in Table 1).

A Comprehensive Map of the EMT State-Associated Kinome

To identify kinases that could be exploited as drug targets to block the EMT and overcome drug resistance, T-test statistics were applied to the dataset of MS intensity values in epithelial (n=7) vs mesenchymal (n=10) HCC cells, identifying 101 kinases, 380 kinase phosphosites, and 938 other phosphoproteins that differed significantly in expression between the two EMT phenotypes (FIGS. 2B and 2D). Remarkably, the protein expression of 66 out of 101 EMT kinases was upregulated in mesenchymal cells (Table 2). These include the clinically important cabozantinib targets AXL (>100-fold) and MET (5-fold), the receptor tyrosine kinase EPHB2 (>100-fold) and the non-receptor kinases FYN, AKT3, CAMK1D, NUAK1 and NUAK2 (all >4-fold, FIG. 2D). The EMT-associated phosphokinome revealed that AXL's kinase activity, but not MET's, was increased in mesenchymal HCC cells, suggesting that AXL plays a more important role in EMT than MET. EPHB2, CAMK1D, FYN and 18 other kinases were also highly activated in mesenchymal HCC cells, indicating that these kinases promote the EMT (FIGS. 2D and 7B). Conversely, epithelial HCC cells showed increased expression of 35 kinases, including the lenvatinib targets FGFR3 and 4 (˜15-fold), and the sorafenib and regorafenib target BRAF (3-fold, FIG. 2D). Other highly upregulated kinases (>4-fold) included NEK3, CDK3, PLK1 and CHEK1 that have important roles in cell cycle and DNA damage response (DDR) signaling. Additionally, activating phosphosites on kinases and specific kinase substrates enriched in the epithelial cell phosphoproteome were identified that confirmed increased activity of the cell cycle through CDK2 and mitogenic signaling through FGFR3/4, BRAF and MAPK1/3 (FIGS. 2D and 7C). These findings confirm that proliferation- and cell cycle-related pathways are specifically upregulated in epithelial cancer cells and downregulated during cancer cell EMT. Collectively, these results represent the first comprehensive proteomics dataset of kinase signaling associated with the HCC cell EMT state.

AXL Drives Reprogramming of the EMT-Associated Kinome

This kinome profiling data revealed that activating phosphorylation sites on AXL and the protein itself are highly enriched in mesenchymal over epithelial HCC cells (FIGS. 2D and 7B). AXL is an important player in cancer cell EMT and the development of tumor metastasis and drug resistance in HCC, as highlighted by the recent success of the AXL and MET inhibitor, cabozantinib, as a second-line treatment for sorafenib-resistant HCCs. However, no detailed proteomics studies of AXL signaling have been published to date. Because such studies may reveal important AXL pathway components that can serve as EMT markers and molecular targets to break drug resistance, RNAi was used to knock down AXL in the FOCUS cell line, a widely used mesenchymal HCC cell model (not shown) (Gujral, T. S., et al. (2014). A noncanonical Frizzled2 pathway regulates epithelial-mesenchymal transition and metastasis. Cell 159, 844-856, incorporated herein by reference in its entirety). Western blot analysis of four EMT marker proteins and qPCR quantification of 43 EMT marker mRNAs confirmed that AXL RNAi induced widespread expression changes indicating reversal of the EMT (not shown). Next, the FOCUS AXL RNAi line was compared to the wild-type (WT) control using kinobead/LC-MS and it was found that 159 kinases and 187 phosphosites on 104 kinases significantly changed in expression (T-Test result, BH-FDR=0.05, n=6 each, FIG. 3A). Importantly, 13 kinases that decreased most (all >4-fold) with AXL-RNAi in FOCUS cells overlapped with the EMT state-associated kinome in the 17 HCC lines (FIG. 3B), among them, for example, the mesenchymal kinases NUAK1 and 2, FYN, EGFR and MET (FIG. 3C).

Concurrently, kinases associated with the epithelial state such as FGFR2/3 and CHEK2 increased in expression in AXL RNAi cells (FIG. 3A). Together, these results highlight the effects of AXL in HCC cell EMT and identify downstream kinases that may play important roles in the EMT and drug response.

FZD2 is a Master Regulator of AXL Expression and EMT-Associated Kinome Rewiring

Having identified various kinases downstream of AXL, the next aim was to identify upstream signaling components that could regulate AXL expression and the EMT in HCC cells. The inventors found previously that AXL mRNA tightly co-expresses with FZD2 mRNA in various cancer cell lines and that this G-protein coupled receptor for WNTSA/B regulates HCC cell EMT via a FYN/STAT3-dependent pathway. To investigate a possible functional connection between FZD2, AXL, and other downstream pathway components, FOCUS FZD2 RNAi cell model was profiled with kinobead/LC-MS. Indeed, FZD2 knockdown affected the expression of 118 kinases, including AXL and several of its effector kinases (T-Test result, BH-FDR=0.05, n=6 each, not shown). Among kinases most affected by AXL and FZD2 RNAi (MS ratio >4-fold), 61% are transcriptional targets of both AXL and FZD2 signaling (FIG. 3B). Importantly, comparing kinases affected by RNAi with those associated with the EMT state in the 17 HCC lines identified nine common kinases, and among those NUAK1 and NUAK2 were particularly sensitive to AXL and FZD2 RNAi (FIGS. 3B and 3C). These results indicate that a FZD2-AXL pathway drives EMT state-associated kinome rewiring in HCC. To investigate if AXL expression is connected to FZD2's activation of FYN and STAT3, STAT3 was knocked down in FOCUS cells and it was found that AXL mRNA levels decreased drastically (FIG. 3D). These results indicate that AXL may be a transcriptional target of STAT3, further supporting the existence of a FZD2-FYN/STAT3-AXL signaling module in HCC cells. It was also found that AXL RNAi in FOCUS cells affects the phosphorylation of STAT3 at its activating site, pY705, identifying a probable feed-forward loop from AXL to STAT3 that reinforces AXL expression (not shown). Collectively, these results integrate AXL into the greater framework of FZD2-regulated HCC cell EMT and reveal additional kinase targets for pharmacological intervention

NUAK1 and NUAK2 Drive AXL Expression and Promote the HCC Cell EMT

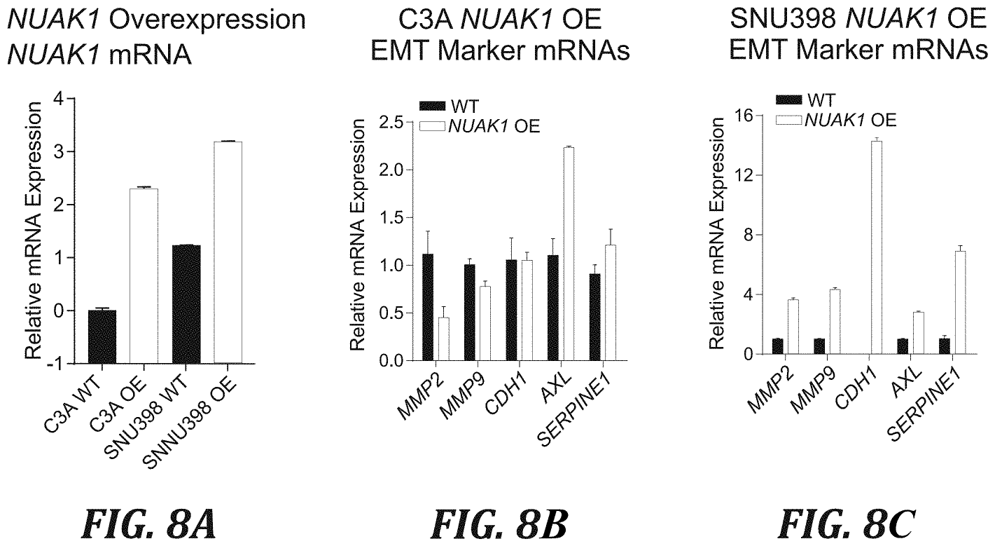

To identify novel drug targets that can reverse the EMT and overcome drug resistance, kinases downstream of the FZD2-FYN/STAT3-AXL signaling module were sought. The nuclear serine/threonine kinases NUAK1 and 2 were tightly associated with the mesenchymal state in the 17 HCC lines and their expression greatly decreased with AXL and FZD2 RNAi (20 to 30-fold, FIG. 3C). NUAK1 is implicated in tumor metastasis and both NUAK1 and NUAK2 were shown to promote tumor cell survival. To test if NUAK1 and 2 are bona fide drivers of the HCC cell EMT, stable FOCUS NUAK1 or NUAK2 RNAi cell lines were generated (not shown); knockdown reduced cell migration by 60-70%, compared to a 25% reduction in FOCUS AXL RNAi cells (FIGS. 3E and 3F), suggesting a prominent role of NUAK1 and 2 in HCC cell EMT. Next, FOCUS NUAK1 and 2 RNAi cells were compared to WT cells using kinobead/LC-MS and it was found that NUAK1 or NUAK2 knockdown affected the expression of 150 and 135 protein kinases, respectively (T test result, BH-FDR=0.05, n=6 each (not shown). Surprisingly, the kinome profiles of NUAK1 and 2 RNAi cells were very similar. The expression of 43 out of 52 highly regulated kinases (MS ratio >4-fold) was affected in both knockdown lines and the LFQ-MS ratios of all affected kinases had a Pearson's r value of 0.91 (FIG. 3G). Strikingly, when AXL RNAi was compared with NUAK1 and 2 RNAi, 85% of highly affected kinases (MS ratio >4-fold) were common between AXL, NUAK1 or NUAK2 RNAi experiments and the MS ratios of all regulated kinases showed a r-value of 0.93 (FIG. 3G). Furthermore, AXL expression levels were greatly decreased by either NUAK1 or NUAK2 RNAi and vice versa (FIG. 3H). This hinted at a positive feedback mechanism where NUAK kinases promote AXL expression or protein stability. Indeed, the qPCR analysis of EMT markers confirmed that AXL, CD44 and MMP2 were decreased in FOCUS NUAK RNAi cells (FIG. 3I). Additionally, ectopic expression of NUAK1 in epithelial-type C3A and SNU398 cells caused at least partial EMT as indicated by increased expression of MMP2 and MMP9, AXL and SERPINE1 (FIGS. 8A-8C). These results confirm that NUAK1 and NUAK2 drive the HCC cell EMT, indicating that these kinases are valuable targets for the development of drugs that reverse the EMT and restore chemosensitivity.

AXL-NUAK1/2 Signaling Promotes Resistance to Cell Cycle Checkpoint Kinase Inhibitors

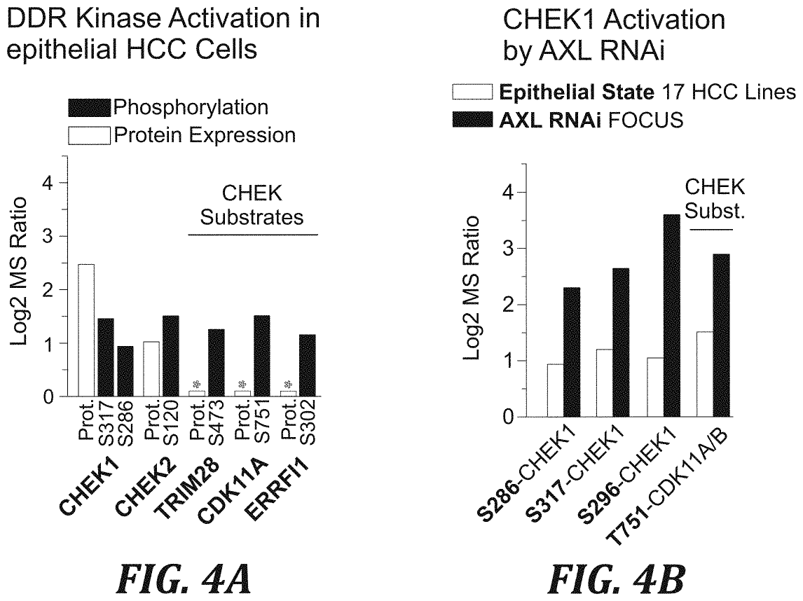

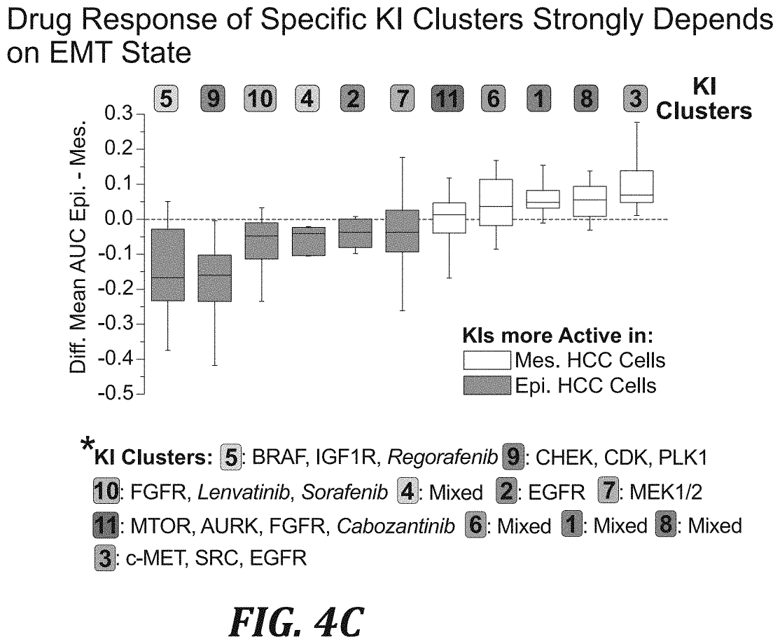

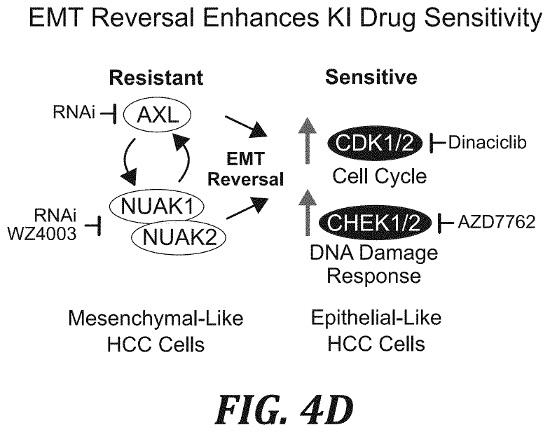

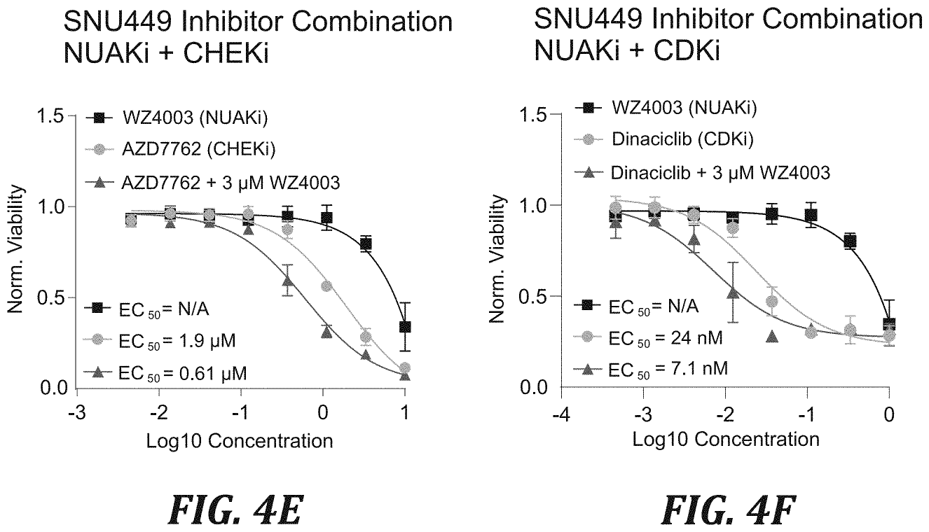

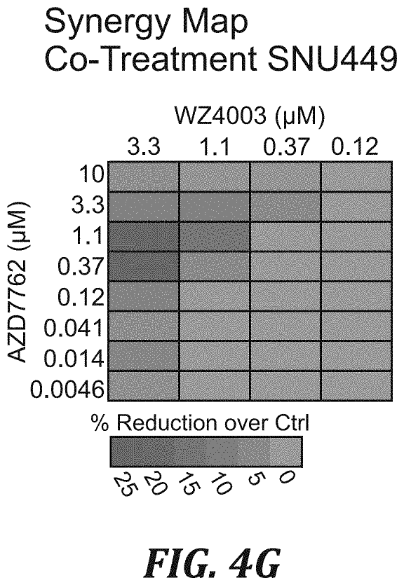

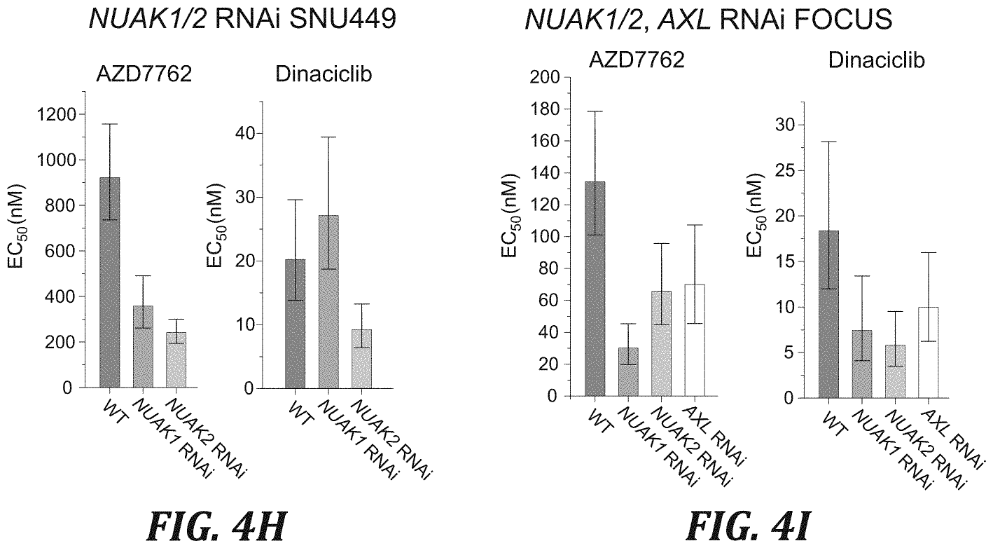

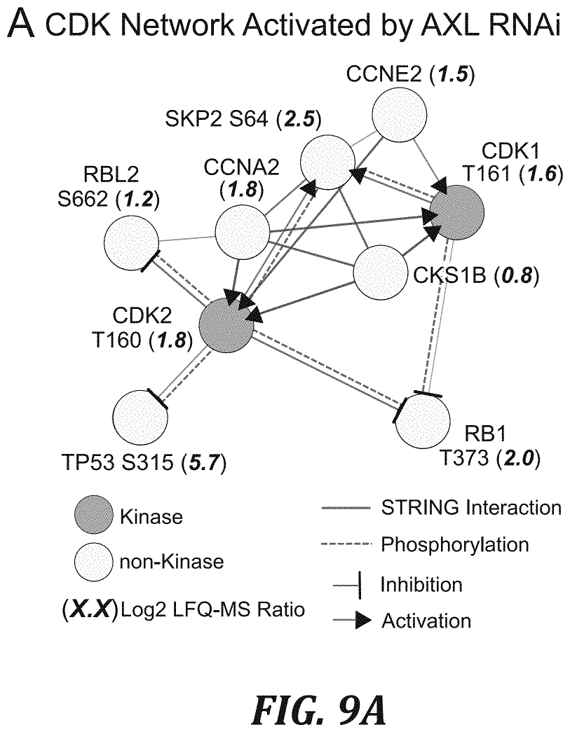

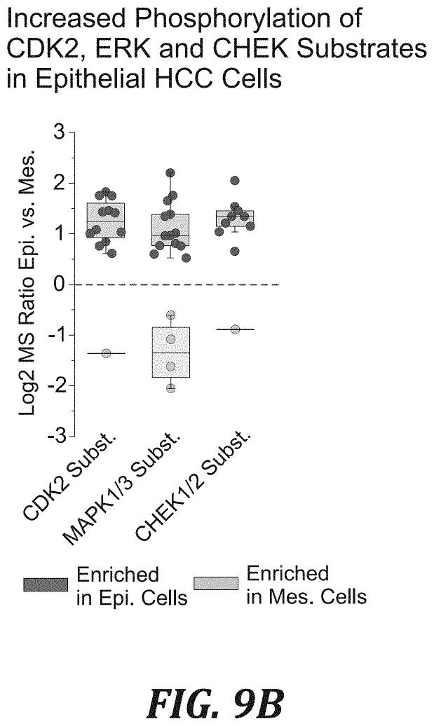

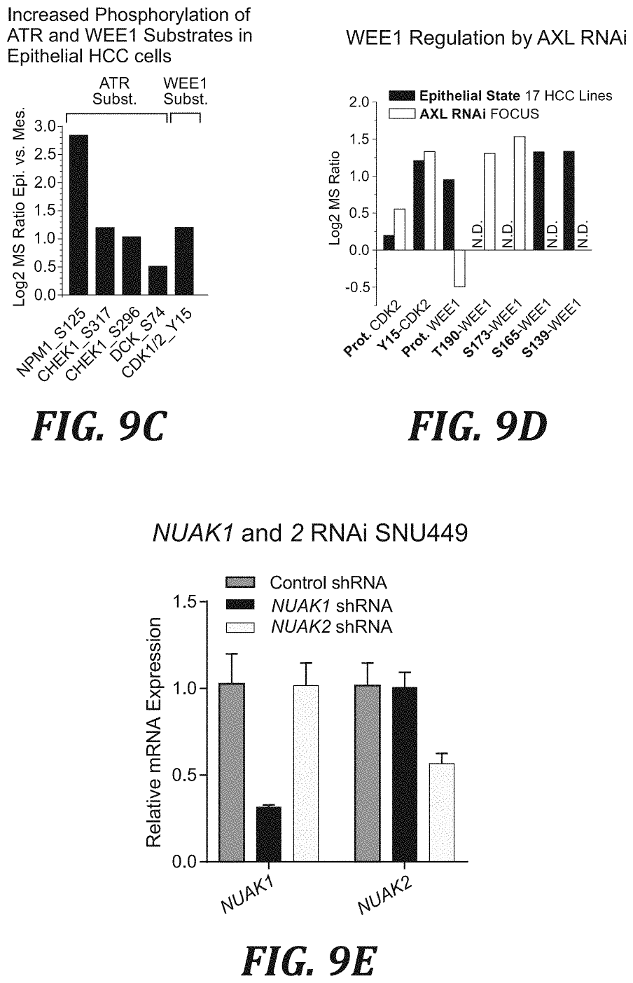

To identify combinatorial treatment strategies targeting mesenchymal and drug resistant HCC cells, the next step was to analyze drug sensitivity pathways that become activated upon AXL and NUAK1/2 inhibition and EMT reversal. It was found that FZD2, AXL and NUAK1/2 RNAi increased expression of FGFR isoforms and activation of mitogenic signaling and cell cycle cues (FIG. 9A). It was also observed increased cell cycle activity among epithelial lines in the 17 HCC line panel (FIG. 9B), that was accompanied by elevated activity of kinases promoting DDR signaling and cell survival under conditions of replication stress. For instance, activation of CHEK1 and 2 along with increased phosphorylation of ATR, CHEK1/2 and WEE1 substrates were observed (FIGS. 4A and 9C). Similarly, AXL RNAi in FOCUS cells activated the DDR kinases CHEK1 and WEE1, increasing phosphorylation of their substrates CDK11B and CDK2 (FIGS. 4B and 9D). These findings suggest that AXL and NUAK1/2 suppress the cell cycle and DDR signaling and protect HCC cells from replication stress. Concurrently, the efficacy of KI drugs that target cell cycle-related kinases was strongly reduced in the mesenchymal fraction of the 17 HCC lines (FIG. 4C). Thus, it was found that the effects of the broadly active CDK, PLK1 and CHEK1/2 inhibitors (KI cluster 9) were among the inhibitor classes most dependent on the EMT state (FIGS. 4C and 5B), indicating that epithelial HCC cell survival may indeed depend on DDR signaling. It was reasoned, therefore, that combinatorial inhibition of AXL or NUAK1/2, as well as cell cycle checkpoint kinases could efficiently kill mesenchymal HCC cells (FIG. 4D). To test this strategy, the mesenchymal and drug resistant SNU449 line was cotreated with the selective NUAK1/2 inhibitor WZ4003 (Banerjee, S., et al. (2014). Characterization of WZ4003 and HTH-01-015 as selective inhibitors of the LKB1-tumour-suppressor-activated NUAK kinases. Biochem J 457, 215-225, incorporated herein by reference in its entirety) and the CHEK1/2 inhibitor AZD7762. Additionally, the CDK inhibitor dinaciclib was used to test if elevated, EMT state-dependent, cell cycle activity translates into increased efficacy of such drugs. Remarkably, cotreatment reproducibly decreased EC₅₀s, i.e. ˜3-fold and ˜2-fold for both CHEK and CDK inhibitors (FIGS. 4E and 4F). This effect was dose-dependent for both AZD7762 and WZ4003 in SNU449 cells, and still significant at 1 μM of WZ4003 (FIG. 4G). These encouraging results led to establishment of SNU449 NUAK1 or NUAK2 RNAi cells (FIG. 9E). Gratifyingly, treating SNU449 NUAK RNAi cells and WT controls with varying doses of AZD7762 and dinaciclib recapitulated the drug co-treatment results (˜3- to 4-fold decrease in EC₅₀s, FIG. 4H). To consolidate these findings, the FOCUS NUAK and AXL RNAi cell lines were tested against AZD7762 and dinaciclib. As in SNU449 cells, NUAK1/2 RNAi FOCUS cells were sensitized to kinase inhibition up to ˜5-fold (FIG. 4I). Together, these results establish, for the first time, that NUAK1 and NUAK2 can regulate HCC cell resistance to targeted therapy.

### DISCUSSION

Described here is a kinome-centric pharmacoproteomics approach integrating kinome activity profiles and drug responses to identify signaling pathways underlying HCC drug sensitivity and resistance. The data indicates that the phospho-activation states of kinases and their interactors are often better predictors of drug response than mRNA or protein expression. Proteome and PTM expression data is, therefore, an important component currently missing in pharmacogenomics biomarker and drug target discovery. It is demonstrated here that the disclosed kinobead/LC-MS platform can measure kinome activity in individual patient HCC's, revealing predictive drug response signatures that might be used to rationally select specific therapies. This approach can be readily applied to other cell line models, organoids, or organotypic slice cultures to identify markers of drug response, classify drug-sensitive and -resistant disease subtypes, and novel drug targets. Reproducible and amenable to lab automation, this platform can be readily scaled up to profile hundreds of clinical tumor samples or cell lines. Importantly, this study provides the first comprehensive map of the kinase-dependent signaling networks that define the mesenchymal and epithelial state in cancer. The quantitative measurements of kinome abundance and phosphorylation sites identified 199 kinases associated with EMT phenotypes, providing a clearer picture of the signaling mechanisms underlying the EMT. Specific signaling modules were identified that promote the mesenchymal drug-resistant phenotype and demonstrated that the disclosed approach can be used to rationally select drug combinations that increase drug sensitivity and cell killing. Finally, the dataset of kinome features and signaling pathways is an important resource for researchers studying kinase inhibitors and kinase-dependent cell signaling.

### “STAR” Methods

TABLE 4
Key resources

| Antibodies | | |
|---|---|---|
| Phospho-Stat3 (Tyr705) | Cell Signaling | Cat# 9145, RRID: |
| (D3A7) Rabbit mAb | Technology | AB_2491009 |
| Reagent or Resource | Source | Identifier |
| Antibodies | | |
| E-Cadherin (24E10) Rabbit mAb | Cell Signaling | Cat# 3195, RRID: |
| | Technology | AB_2291471 |
| Mouse Anti-Occludin | BD Biosciences | Cat# 611091, RRID: |
| | | AB_398404 |
| Mouse Anti-Vimentin Antibody, clone V9 | Millipore | Cat# MAB3400, |
| | | RRID: AB_94843 |
| Mouse Anti-beta-Actin Monoclonal | Sigma-Aldrich | Cat# A1978, RRID: |
| Antibody, Unconjugated, Clone AC-15 | | AB_476692 |
| GAPDH (G-9) Antibody, Mouse | Santa Cruz | Cat# sc-365062, |
| | Biotechnology | RRID: |
| | | AB_10847862 |
| Phospho-p44/42 MAPK (Erk1/2) | Cell Signaling | Cat# 4370, RRID: |
| (Thr202/Tyr204) (D13.14.4E) Rabbit mAb | Technology | AB_2315112 |
| Ax1 (C89E7) Rabbit mAb | Cell Signaling | Cat# 8661, RRID: |
| | Technology | AB_11217435 |
| Bacterial and Virus Strains | | |
| Biological Samples | | |
| 13 Paired primary HCC and normal adjacent | Laboratory of Raymond | |
| liver specimens, Human | S. Yeung, University of | |
| | Washington | |
| Chemicals, Peptides, and Recombinant Proteins | | |
| Kinobead affinity capture reagents | Laboratory of Dustin J. | Compounds #1, 2, 3 |
| | Maly, University of | 4, 5, 6 and 7 |
| | Washington (Golkowski, | |
| | M., et al. (2017a). | |
| | Methods Mol Biol 1636, | |
| | 105-117) | |
| Dinaciclib | Selleckchem | Cat#: S2768, CAS: |
| | | 779353-01-4 |
| AZD7762 | Selleckchem | Cat#: S1532, CAS: |
| | | 860352-01-8 |
| WZ4003 | Tocris Bioscience | Cat#: 5177, CAS: |
| | | 1214265-58-3 |
| Lapatinib | Selleckchem | Cat#: S2111, CAS: |
| | | 231277-92-2 |
| Custom-Lys/-Arg DMEM | Caisson Labs | Cat#: DML07 |
| Custom-Lys/-Arg RPMI-1640 | Caisson Labs | Cat#: RPL01 |
| L-ARGININE:HCL (13C6, 99%; 15N4, 99%) | Cambridge Isotope | Cat#: CNLM-539- |
| | Laboratories | H-PK |
| L-LYSINE:2HCL (13C6, 99%; 15N2, 99%) | Cambridge Isotope | Cat#: CNLM-291- |
| | Laboratories | H-PK |
| Seradigm Fetal Bovine Serum (FBS) | VWR Life Science | Cat#: 97068-085 |
| Dialyzed FBS, 10,000 Da Cutoff | Sigma-Aldrich | |
| Lysyl Endopeptidase, Mass Spectrometry | Wako | Cat#: 125-05061 |
| Grade (Lys-C) | | |
| Pierce Trypsin Protease, MS Grade | Thermo Fisher Scientific | Cat#: 90058 |
| PHOS-select iron affinity gel | Sigma-Aldrich | Cat#: P9740 |
| Ni-NTA | Superflow resin Quiagen | Cat#: 30410 |
| Critical Commercial Assays | | |
| Kinase Inhibitor High Throughput Screen | QUELLOS HTS Facility, | http://depts.washing- |
| | University of Washington | ton.edu/iscrm/ |
| | | quellos/ |
| RNeasy Mini Kit | Quiagen | Cat#: 74104 |
| CellTiter-Glo 2.0 Assay | Promega | Cat#: G9241 |
| Deposited Data | | |
| All MS raw files and MaxQuant output files | MassIVE Repository of | |
| | the University of | |
| | California, San Diego | |
| Experimental Models: Cell Lines | | |
| Human: SNU449, <10 Passages | ATCC | Cat#: CRL-2234 |
| Human: HuH-7, <10 Passages | JRCB Cell Bank | Cat#: JCRB0403 |
| Human: SNU878, <10 Passages | Korean Cell Line Bank | Cat#: 00878 |
| | (KCLB) | |
| Human: FOCUS, <10 Passages | Laboratory of J. Wands, | N/A |
| | Brown University, (He, | |
| | Let al. (1984). In Vitro | |
| | 20, 493-504) | |
| Human: C3A, <10 Passages | ATCC | Cat#: CRL-10741 |
| Human: HepG2, <10 Passages | ATCC | Cat#: HB-8065 |
| Human: SNU398, <10 Passages | ATCC | Cat#: CRL-2233 |
| Human: PLC/PRF/5, <10 Passages | ATCC | Cat#: CRL-8024 |
| Human: Hep3B2.1-7, <10 Passages | ATCC | Cat#: HB-8064 |
| Human: SKHep1, <10 Passages | ATCC | Cat#: HTB-52 |
| Human: SNU475, <10 Passages | ATCC | Cat#: CRL-2236 |
| Human: SNU387, <10 Passages | ATCC | Cat#: CRL-2237 |
| Human: SNU423, <10 Passages | ATCC | Cat#: CRL-2238 |
| Human: NCI-H684, <10 Passages | Korean Cell Line Bank | Cat#: 90684 |
| | (KCLB) | |
| Human: SNU761, <10 Passages | Korean Cell Line Bank | Cat#: 00761 |
| | (KCLB) | |
| Human: SNU886, <10 Passages | Korean Cell Line Bank | Cat#: 00886 |
| | (KCLB) | |
| Human: JHH4, <10 Passages | JRCB Cell Bank | Cat#: JCRB0435 |
| Human: JHH6, <10 Passages | JRCB Cell Bank | Cat#: JCRB1030 |
| Human: HCT-116, <10 Passages | ATCC | Cat#: CCL-247 |
| Human: SH-SY5Y, <10 Passages | ATCC | Cat#: CRL-2266 |
| Human: U-2 OS, <10 Passages | ATCC | Cat#: HTB-96 |
| Human: Du4475, <10 Passages | ATCC | Cat#: HTB-123 |
| Human: HL-60, <10 Passages | ATCC | Cat#: PTS-CCL-240 |
| Human: Jurkat, <10 Passages | ATCC | Cat#: PTS-TIB-152 |
| Human: K562, <10 Passages | ATCC | Cat#: CCL-243 |
| Experimental Models: Organisms/Strains | | |
| Oligonucleotides | | |
| GIPZ STAT3 shRNA | Dharmacon | Cat#: RH54531- |
| | | EG6774 |
| GIPZ FYN shRNA | Dharmacon | Cat#: RHS4531- |
| | | EG2534 |
| GIPZ FZD2 shRNA | Dharmacon | Cat#: RHS4531- |
| | | EG2535 |
| GIPZ AXL shRNA | Dharmacon | Cat#: RHS4531- |
| | | EG558 |
| GIPZ NUAK1 shRNA | Dharmacon | Cat#: RH54531- |
| | | EG9891 |
| GIPZ NUAK2 shRNA | Dharmacon | Cat#: RHS4531- |
| | | EG81788 |
| Recombinant DNA | | |
| NUAK1 in pReceiver-Lv242 | GeneCopoeia | Cat#: EX-M0778- |
| | | Lv242, NM_014840.2 |
| Software and Algorithms | | |
| MaxQuant/Andromeda, v1.5.2.8 | biochem.mpg.de/ | (Cox et al., (2011) |
| | 5111795/maxquant | Journal of proteome |
| | | research 10, 1794- |
| | | 1805) |
| Perseus, v1.5.6.0 | biochem.mpg.de/ | (Tyanova etal., |
| | 5111810/perseus | (2016) Nat Methods |
| | | 13, 731-740) |
| R-package, gplots v3.0.1, gplots::heatmap.2 | rdocumentation.org/pack- | N/A |
| | ages/gplots/versions/3.0.1 | |
| R-package, Bioconductor: fgsea | bioconductor.org/pack- | biorxiv.org/content/ |
| | ages/release/bioc/html/ | early/2016/06/20/ |
| | fgsea.html | 060012 |
| GraphPad Prism, V7 | graphpad.com/ | N/A |
| STRING, database, enrichment analysis | string-db.org/ | (Szklarczyk et al., |
| | | (2015) Nucleic acids |
| | | research 43, D447- |
| | | 452) |
| BioGRID, database | thebiogrid.org/ | (Chatr-Aryamontri |
| | | et al., (2017) Nucleic |
| | | acids research 45, |
| | | D369-D379) |
| PhosphoSite Plus, database | phosphosite.org/ | (Hornbeck et al., |
| | homeAction | (2015) Nucleic acids |
| | | research 43, D512- |
| | | 520) |
| Reactome, database | reactome.org/ | (Fabregat et al., |
| | | (2018) Nucleic acids |
| | | research 46, D649- |
| | | D655) |
| BioVenn | biovenn.nl/ | (Hulsen et al., |
| | | (2008) BMC |
| | | Genomics 9, 488) |
| KinMap | kinhub org/kinmap/ | Eid S., et al. (2017). |
| | | BMC |
| | | Bioinformatics, |
| | | 18:16. |

### Experimental Model and Subject Details

Cell Lines and Tissue Culture Conditions

C3A, HepG2, SNU398, PLC/PRF/5, Hep3B2.1-7, SKHep1, SNU475, SNU387, SNU423 and SNU449 cell lines were purchased from the American Type Culture Collection (ATCC). NCI-H684, SNU761, SNU886 and SNU878 were purchased from the Korean Cell Line Bank (KCLB). JHH4, JHH7 and HuH-7 cells were purchased form the JRCB Cell Bank. FOCUS cells were obtained from the Laboratory of J. Wands, Brown University (He, L., et al. (1984). Establishment and characterization of a new human hepatocellular carcinoma cell line. In Vitro 20, 493-504, incorporated herein by reference in its entirety). All cells were grown at 37° C. under 5% CO2, 95% ambient atmosphere. Fifteen cryofrozen cell stocks were generated from the original vial from the cell bank (Passage 3). Experiments were performed with cells at <10 passages from the original vial. All cell media used were supplemented with 100× penicillin-streptomycin-glutamine (Thermo Fisher Scientific, Waltham, Mass.). FOCUS and HuH-7 cells were grown in Dulbecco's minimum essential medium (DMEM) supplemented with 10% FBS (VWR Life Science, Seradigm). C3A, HepG2, SNU398, PLC/PRF/5, Hep3B2.1-7, SKHep1, SNU475, SNU387, SNU423 and SNU449 were grown in the ATCC-recommended medium. JHH4 cells were grown in Eagle's minimum essential medium (MEM), JHH6 cells in William's E medium and NCI-H684, SNU761, SNU886 and SNU878 in RPMI 1640 medium all supplemented with 10% FBS. Cells were harvested when reaching 90% confluency.

Human HCC and Normal Adjacent Liver Specimens

Primary human HCCs with paired non-tumor livers were obtained from patients undergoing liver resection at the University of Washington Medical Center (Seattle, Wash., USA). All patients in this study prospectively consented to donate liver tissue for research under the Institutional Review Board protocols #1852. Once the specimens were collected under the direction of Pathology representatives, they were snap-frozen in liquid nitrogen and stored at −80° C. until further processing. Patient samples were characterized as provided in Table 5.

TABLE 5
Patient samples

| | | | | | Histo- | |
|---|---|---|---|---|---|---|
| | | | Tumor | | logical | Prior |
| Case # | Age | Gender | Description | Etiology | Grade | Treatment |
| 1 | 56 | M | HCC, 2.5 cm | HBV | G2 | None |
| 2 | 77 | F | Mixed | HCV | G2 | None |
| | | | ICC/HCC, | | | |
| | | | 3.7 cm | | | |
| 3 | 61 | M | HCC, 8 cm | HCV | G2 | Recurrent |
| | | | | | | after |
| | | | | | | Resection |
| 4 | 67 | M | HCC, 3 cm | EtOH | G2 | Recurrent |
| | | | | Cirrhosis | | after |
| | | | | | | Resection |

### Methods

RNAi Knockdown Experiments

All lentiviral vectors encoding different shRNAs (STATS, FYN, FZD2, AXL, NUAK1 and 2) in a pGIPZ vector were purchased from OpenBiosystems (Dharmacon, Lafayette, Colo.). Cell lines were transfected with shRNA constructs using Lipofectamine 2000 (Invitrogen, Carlsbad, Calif.) and 48 h post-transfection selected with 4 μg/ml puromycin (Invitrogen). The clones were sorted by FACS and screened for target mRNA knockdown by Western blot or qPCR analysis (see ‘Western blot analysis and antibodies’ and ‘Quantitative real-time PCR (qPCR) analysis of mRNA expression’ below). Stable cell lines were maintained in DMEM (see ‘Cell lines and cell culture conditions’) supplemented with 2 μg/ml puromycin.

Ectopic Expression of NUAK1

The expression construct encoding for full length NUAK1 (NM_014840.2) in a lentiviral plasmid (pReceiver-Lv242) was purchased from Genecopoeia (Rockville, Md.). Cell lines were transfected with NUAK1 plasmid construct using Lipofectamine 2000 (Invitrogen, Carlsbad, Calif.) and 48 h post-transfection selected in 4 μg/ml puromycin (Invitrogen). Stable cell lines were maintained in DMEM (see ‘Cell lines and tissue culture conditions’) supplemented with 2 μg/ml puromycin.

Western Blot Analysis and Antibodies

Antibodies used were anti-phospho-Stat3 (Tyr705) (Cell signaling Technology, Cat #9145), anti-E-cadherin (Cell signaling Technology, Cat #3195), anti-Occludin (BD Transduction Laboratories, Cat #611091), anti-Vimentin (Millipore, Cat #CS207806), anti-β-Actin (Sigma, Cat #A1978), anti-GAPDH (Santacruz Biotechnology, Cat #Sc-365062), anti-phospho-p44/42 MAPK (Erk1/2) (Thr202/Tyr204) (Cell signaling Technology, Cat #4370), anti-Axl (C89E7) (Cell signaling Technology, Cat #8661). Briefly, cells were rinsed in phosphate buffered saline (PBS) and lysed in lysis buffer (20 mM Tris-HCl, 150 mM NaCl, 1% Triton X-100 (v/v), 2 mM EDTA, pH 7.8 supplemented with 1 mM sodium orthovanadate, 1 mM phenylmethylsulfonyl fluoride (PMSF), 10 μg/ml aprotinin, and 10 μg/ml leupeptin). Protein concentrations were determined using the BCA protein assay (Pierce, Rockford, Ill.) and immunoblotting experiments were performed using standard procedures. For quantitative immunoblots, primary antibodies were detected with IRDye 680-labeled goat-anti-rabbit IgG or IRDye 800-labeled goat-anti-mouse IgG (LI-COR Biosciences, Lincoln, Nebr.) at 1:5000 dilution. Bands were visualized and quantified using an Odyssey Infrared Imaging System (LI-COR Biosciences).

Quantitative Real-Time PCR (qPCR) Analysis of mRNA Expression

Cells were seeded in 6-well plates 24 h prior to isolation of total RNA using a RNeasy Mini Kit (QIAGEN, Santa Clara, Calif.). mRNA levels for EMT-related genes were determined using the validated primer sets (SA Biosciences Corporation, Frederick, Md.). Briefly, 1 μg of total RNA was reverse transcribed into first strand cDNA using an RT² First Strand Kit (SA Biosciences). The resulting cDNA was subjected to qPCR using human gene-specific primers for EMT-associated genes, and two housekeeping genes (GAPDH and ACTB). The qPCR reaction was performed with an initial denaturation step of 2 min at 95° C., followed by 5 s at 95° C. and 30 s at 60° C. for 40 cycles using a Biorad CFX384 system (Biorad, Hercules, Calif.). The mRNA levels of each gene were normalized relative to the mean levels of the two housekeeping genes and compared with the data obtained from unstimulated, serum-starved cells using the 2-ΔΔCt method. According to this method, the normalized level of a mRNA, X, is determined using Equation 1:

X=2^{-Ct(GOI)}/2^{-Ct(CTL)} (1)

where Ct is the threshold cycle (the number of the cycle at which an increase in reporter fluorescence above a baseline signal is detected), GOI refers to the gene of interest, and CTL refers to a control housekeeping gene. This method assumes that Ct is inversely proportional to the initial concentration of mRNA and that the amount of product doubles with every cycle.

Kinetic Wound Healing Assay

A wound healing assay was used to study the effect of AXL and NUAK1/2 RNAi knockdown on FOCUS cell migration and to score the cell motility of the 17 wt HCC cell lines. Briefly, cells were plated on 96-well plates (Essen Image Lock, Essen Bioscience, Ann Arbor, Mich.), and a wound was scratched with a wound scratcher (Essen Instruments). Wound confluence was monitored with Incucyte Live-Cell Imaging System and software (Essen Instruments). Wound closure was observed every 2 hours for 24-72 hours by comparing the mean relative wound density of at least three biological replicates in each experiment.

High Throughput Growth Inhibition Assay

The high throughput growth inhibition assay was performed by the QUELLOS high throughput screening facility of the University of Washington (iscrm.uw.edukesearch/core-resources/quellos-high-throughput-screening-core/) using 299 compounds of a Selleckchem kinase inhibitor Library (selleckchem.com/screening/kinase-inhibitor-library.html, Selleckchem, Houston, Tex.). Briefly, the assay was performed in a 384-well plate format in biological duplicate. Compounds were applied at 7 different concentrations ranging from 10 μM to 10 nM and cell viability was measured after 72 h of incubation using the CellTiter-Glo 2.0 assay (Promega, Madison, Wis.).

Inhibitor Co-Treatment and AXL/NUAK RNAi Growth Inhibition

1800 cells/well were seeded onto white flat bottom half area 96-well plates (Greiner Bio-One, Kremsmuenster, AT) in 50 μl of growth medium and allowed to attach in an incubator for 24 h. Then the drugs in DMSO and/or DMSO vehicle controls as 11× solutions in growth medium were added to a total volume of 55 μl and 0.1% DMSO final. The cells were grown in an incubator for another 72 h. Then, 55 μl of CellTiter-Glo 2.0 (Promega, Madison, Wis.) reagent/well were added according to the manufacturer's instructions and luminescence was quantified with a SpectraMax 190 plate reader (Molecular Devices, San Jose, Calif.). The CHEK1/2 inhibitor AZD7762 (Selleckchem, Houston, Tex.) and the NUAK1/2 inhibitor WZ4003 (Tocris Bioscience, Minneapolis, Minn.) were applied at 8 different concentrations between 10 μM and 4.6 nM (3-fold dilution steps) and Dinaciclib (Selleckchem) was applied at 8 different concentrations between 1 μM and 0.5 nM (3-fold dilution steps). Experiments were performed in four biological replicates. Growth inhibition curves were fitted using the GraphPad Prism software package (V5.0a) with a least-squares nonlinear regression model for curve fitting (One site-Fit log IC50 function).

Protein Extraction from Human HCC and Normal Adjacent Liver Specimens

Frozen tumor specimens of ca. 100 mg wet weight were ground into a fine powder using the CryoGrinder Kit from OPS Diagnostics (Lebanon, N.J.). The powder was then added to ice cold mod. RIPA buffer containing phosphatase and protease inhibitors (see ‘Kinase affinity enrichment and on-bead digestion’ below), vortexed 10 times at max. speed and clarified at 21,000 rcf and 4° C. for 20 min. Protein yields from specimens ranged from 5% to 10% depending on the degree of fibrosis.

IMAC Phosphopeptide Enrichment

IMAC phosphopeptide enrichment was performed according to the published protocol (in-tube batch version) with the following minor modifications (Villen, J., and Gygi, S. P. (2008). The SCX/IMAC enrichment approach for global phosphorylation analysis by mass spectrometry. Nature protocols 3, 1630-1638, incorporated herein by reference in its entirety). 20 μl of a 50% IMAC bead slurry composed of 1/3 commercial PHOS-select iron affinity gel (Sigma Aldrich, St Louis, Mo.), 1/3 in-house made Fe³⁺-NTA Superflow agarose and 1/3 in-house made Ga³⁺-NTA Superflow agarose was used for phosphopeptide enrichment (Ficarro, S. B., et al. (2009). Magnetic bead processor for rapid evaluation and optimization of parameters for phosphopeptide enrichment. Analytical chemistry 81, 4566-4575, incorporated herein by reference in its entirety). The IMAC slurry was washed three times with 10 bed volumes of 80% aq. ACN containing 0.1% TFA and phosphopeptide enrichment was performed in the same buffer.

Peptide and Phosphopeptide Desalting with StageTips

Peptides and phosphopeptides were desalted using C18 StageTips according to the published protocol with the following minor modifications for phosphopeptides (Rappsilber, J., et al. (2007). Protocol for micro-purification, enrichment, pre-fractionation and storage of peptides for proteomics using StageTips. Nature protocols 2, 1896-1906, incorporated herein by reference in its entirety). After activation with 50 μl methanol and 50 μl 80% aq. ACN containing 0.1% TFA the StageTips were equilibrated with 50 μl 1% aq. formic acid. Then the peptides that were reconstituted in 50 μl 1% aq. formic acid were loaded and washed with 50 μl 1% aq. formic acid. The use of 1% formic acid instead of 5% aq. ACN containing 0.1% TFA reduces the loss of highly hydrophilic phosphopeptides.

Preparation of Optimized Kinobead Mixture

The seven kinobead affinity reagents used were synthesized in-house as described previously (Golkowski, M., et al. (2014). Rapid profiling of protein kinase inhibitors by quantitative proteomics. MedChemComm 5, 363-369; Golkowski, M., et al. (2017). Kinobead and Single-Shot LC-MS Profiling Identifies Selective PKD Inhibitors. Journal of proteome research 16, 1216-1227; and Golkowski, M., et al. (2020). Kinobead/LC-MS Phosphokinome Profiling Enables Rapid Analyses of Kinase-Dependent Cell Signaling Networks. Journal of proteome research 19, 1235-1247, each of which is incorporated herein by reference in its entirety). For optimal coverage of the human kinome an optimized mixture of the seven kinobead reagents was prepared as previously described (Golkowski, M., et al. (2020). Journal of proteome research 19, 1235-1247). Briefly, 1 ml of reagent 1, 0.5 ml of reagents 2, 3 and 7, respectively, and 0.25 ml of reagents 4, 5 and 6, respectively, were mixed to yield 3.25 ml of the complete kinobead mixture. All reagents were a 50% slurry in 20% aq. ethanol.

Kinase Affinity Enrichment and On-Bead Digestion

Kinase affinity enrichment and on-bead digestion was performed as previously described (Golkowski, M., et al. (2020). Journal of proteome research 19, 1235-1247). Briefly, three micro tubes containing 35 μl of a 50% slurry of the in-house-made, optimized kinobead mixture in 20% aq. ethanol were prepared for each pulldown experiment. The beads were washed twice with 300 μl modified RIPA buffer (50 mM Tris, 150 mM NaCl, 0.25% Na-deoxycholate, 1% NP-40, 1 mM EDTA and 10 mM NaF, pH 7.8). 1 mg of protein extract in mod. RIPA buffer containing HALT protease inhibitor cocktail (100×, Thermo Fisher Scientific, Waltham, Mass.) and phosphatase inhibitor cocktail II and III (100×, Sigma-Aldrich, St Louis, Mo.) were added to the first tube. The mixture was incubated on a tube rotator for 1h at 4° C. and then the beads were spun down rapidly at 2000 rpm on a benchtop centrifuge (5s). The supernatant was pipetted into the next tube with kinobeads for the second round of affinity enrichment. The procedure was repeated once more for a total of three rounds of affinity enrichment. After removal of the supernatant, the beads were rapidly washed twice with 300 μl of ice-cold mod. RIPA buffer and three times with 300 μl ice-cold tris-buffered saline (TBS, 50 mM tris, 150 mM NaCl, pH 7.8) to remove detergents. 100 μl of the denaturing buffer (20% trifluoroethanol (TFE) (Wang, H., et al. (2005). Development and evaluation of a micro- and nanoscale proteomic sample preparation method. Journal of proteome research 4, 2397-2403, incorporated herein by reference in its entirety), 25 mM Tris containing 5 mM tris(2-carboxyethyl)phosphine hydrochloride (TCEP*HCl) and 10 mM chloroacetamide (CAM), pH 7.8), were added and the slurry vortexed at low speed briefly. At this stage, kinobeads from the three tubes are combined and heated at 95° C. for 5 min. The mixture was diluted 2-fold with 25 mM triethylamine bicarbonate (TEAB), the pH adjusted to 8-9 by addition 1 N aq. NaOH; 5 μg LysC were added and the mixture agitated on a thermomixer at 700 rpm at 37° C. for 2 h. Then 5 μg MS-grade trypsin (Thermo Fisher Scientific, Waltham, Mass.) were added, and the mixture agitated on a thermomixer at 700 rpm at 37° C. overnight. 600 μl of 1% formic acid was added and the mixture acidified by addition of another 6 μl of formic acid to yield 1.2 ml peptide solution in total. An aliquot of 120 μl (10%) of the peptide solution was desalted using StageTips and analyzed in single nanoLC-MS/MS runs for protein quantification. The remaining peptide solution (90%) was dried under vacuum at RT on a SpeedVac. 300 μl of 70% aq. ACN+0.1% TFA was added to each tube, the mixture vortexed, and sonicated in a bath sonicator until dried peptide residue dissolved. In case the dried residue could not be fully resuspended, additional 0.1% aq. TFA can be added in 10 μl increments until dissolved. The solution was subjected to IMAC phosphopeptide enrichment protocol and desalted using StageTips (see ‘IMAC phosphopeptide enrichment’ and ‘Peptide and phosphopeptide desalting with StageTips’ above).

nanoLC-MS/MS Analyses

The LC-MS/MS analyses were performed as described previously with the following minor modifications (Golkowski, M., et al. (2017). Kinobead and Single-Shot LC-MS Profiling Identifies Selective PKD Inhibitors. Journal of proteome research 16, 1216-1227; and Golkowski, M., et al. (2020). Kinobead/LC-MS Phosphokinome Profiling Enables Rapid Analyses of Kinase-Dependent Cell Signaling Networks. Journal of proteome research 19, 1235-1247). Peptide samples were separated on a Thermo-Dionex RSLCNano UHPLC instrument (Sunnyvale, Calif.) using 20 cm long fused silica capillary columns (100 μm ID) packed with 3 μm 120 Å reversed phase C18 beads. For whole peptide samples the LC gradient was 120 min long with 10-35% B at 300 nL/min. For phosphopeptide samples the LC gradient was 120 min long with 3-30% B at 300 nL/min LC solvent A was 0.1% aq. acetic acid and LC solvent B was 0.1% acetic acid, 99.9% acetonitrile. MS data was collected with a Thermo Fisher Scientific Orbitrap Elite (kinobead-MS experiments, global phosphoproteomics analyses) or Orbitrap Fusion Lumos Tribrid (global proteome analyses) spectrometer. Data-dependent analysis was applied using Top15 selection with CID fragmentation.

Computation of MS Raw Files

Data .raw files were analyzed by MaxQuant/Andromeda (Cox, J., et al. (2011). Andromeda: a peptide search engine integrated into the MaxQuant environment. Journal of proteome research 10, 1794-1805, incorporated herein by reference in its entirety) version 1.5.2.8 using protein, peptide and site FDRs of 0.01 and a score minimum of 40 for modified peptides, 0 for unmodified peptides; delta score minimum of 17 for modified peptides, 0 for unmodified peptides. MS/MS spectra were searched against the UniProt human database (updated Jul. 22, 2015). MaxQuant search parameters: Variable modifications included Oxidation (M) and Phospho (S/T/Y). Carbamidomethyl (C) was a fixed modification. Max. missed cleavages was 2, enzyme was Trypsin/P and max. charge was 7. The MaxQuant “match between runs” feature was enabled. The initial search tolerance for FTMS scans was 20 ppm and 0.5 Da for ITMS MS/MS scans.

MaxQuant Output Data Processing

MaxQuant output files were processed, statistically analyzed and clustered using the Perseus software package v1.5.6.0 (Tyanova, S., Temu, T., Sinitcyn, P., Carlson, A., Hein, M. Y., Geiger, T., Mann, M., and Cox, J. (2016). The Perseus computational platform for comprehensive analysis of (prote)omics data. Nat Methods 13, 731-740, incorporated herein by reference in its entirety). Human gene ontology (GO) terms (GOBP, GOCC and GOMF) were loaded from the ‘Perseus Annotations’ file downloaded on 01.08.2017. Expression columns (protein and phosphopeptide intensities) were log 2 transformed and normalized by subtracting the median log 2 expression value from each expression value of the corresponding data column. Potential contaminants, reverse hits and proteins only identified by site were removed. Reproducibility between LC-MS/MS experiments were analyzed by column correlation (Pearson's r) and replicates with a variation of r>0.25 compared to the mean r-values of all replicates of the same experiment (cell line or knockdown experiment) were considered outliers and excluded from the analyses. Data imputation was performed using a modeled distribution of MS intensity values downshifted by 1.8 and having a width of 0.2. For statistical testing of significant differences in expression, a two-sample Student's T-test with Benjamini-Hochberg correction for multiple hypothesis testing was applied (FDR=0.05). For statistical testing of the 17 HCC cell line data (EMT state-association) all biological replicates were used. For MS protein intensities this was n=42 (epithelial cells) and n=60 (mesenchymal cells). For MS phosphopeptide intensities this was n=56 (epithelial cells) and n=91 (mesenchymal cells).

Pharmacoproteomic AUC-MS Intensity Correlation

Mean LFQ-MS intensities values for each of the 17 cell lines were calculated using the imputed MS intensity data. All proteomics features (n=13935) were correlated with each compound's AUC value across the cell line panel (n=17) using the Pearson's correlation coefficient resulting in a 13935×299 matrix of r-values (not shown). To rank proteomics feature the resulting r-values for each kinase inhibitor were sorted from low to high where negative r-values correspond to drug sensitivity (low AUC—high MS intensity) and positive r-values correspond to drug resistance (high AUC-high MS intensity).

Kinome-GSEA Analysis with Reactome Pathways

To obtain signaling pathways for GSEA analyses, the mapped identifier files ‘NCBI2Reactome_All_Levels.txt’ and ‘ReactomePathwaysRelation.txt’ from Reactome.org (downloaded 22 Oct. 2018) were used. Pathways from the highest hierarchical levels were removed to exclude non-specific pathways. Subsequently, a regular expression match for patterns containing “kinase”, “signal”, “cell cycle”, “migration”, “cancer”, “dna repair”, “mitos” and “mitot” was used to extract 327 cancer relevant pathways (‘Reactome_Pathways’). Member genes of each pathway were mapped to unique identifiers to allow addition of phosphopeptide data. The Pearson correlation coefficients r from correlating drug response with MS intensities of kinome features (see ‘Pharmacoproteomic AUC-MS intensity correlation analyses’ above) were −(x) transformed for GSEA analyses, as the GSEA algorithm ranks features by their correlation with drug response. The Bioconductor package, fgsea (Sergushichev, A. (2016) was used. An algorithm for fast preranked gene set enrichment analysis using cumulative statistic calculation. bioRxiv, doi.org/10.1101/060012), with parameters: minSize=10, maxSize=500, gseaParam=2 and nperm=10000 to compute p-values and enrichment scores, including corrections for multiple hypothesis testing (BH FDR=0.1).

Classification of KI Drugs by Correlation-Clustering of Kinome-GSEA wNES Scores

For hierarchical clustering of compounds based on Reactome pathway analysis (FIG. 7A), FDR-weighted Reactome pathway normalized enrichment scores (wNES=NES*(1-FDR)) were extracted for each compound (N=299). Pairwise Pearson correlation coefficients (r) for wNES values for all compounds were calculated resulting in a 299×299 matrix of r-values. Clustering of the Pearson's correlation coefficients using R (gplots::heatmap.2) identified 11 major groups (FIG. 4A). For these 11 major groups the mean wNES values was calculated for 34 cancer-relevant, non-redundant Reactome pathways representative of the larger panel of Reactome pathways (FIG. 2A). These 34 pathways were selected for having the largest difference in mean wNES across all 11 KI drug clusters among Reactome terms in the same overarching pathway theme. The result is a 11×34 matrix of mean wNES values (FIG. 2A) that was subjected to hierarchical clustering using R (gplots::heatmap.2).

Kinome-GSEA in Clinical HCC Specimens and Correlation of Pathway Signatures

The difference of the mean imputed MS intensity values for the protein and phosphopeptide expression kinobead LFQ-MS data tumor vs. normal adjacent liver were calculated and all proteomics features according to this difference of the mean (positive values was enriched in tumor, negative values was enriched in) were ranked. The kinome-GSEA analysis was then applied and FUR-weighted Reactome pathway enrichment scores (wNES values, see ‘Kinome-GSEA analysis with Reactome pathways’) were calculated. Pearson correlation coefficients were then calculated, correlating the wNES values of all 299 KI drugs with the wNES values of the four HCC tumor/NAL pairs. High r-value then indicate enrichment of a KI pathway signature in tumors, and negative values their enrichment in NAL.

Functional Phosphosites and Kinase-Substrate Relationships

To determine the biological function of a phosphorylation site and the kinase-substrate relationship of a given phosphorylation site the PhosphoSite Plus datasets ‘Regulatory_Sites’ and ‘Kinase_Substrate_Dataset’ were searched against the 15 amino acid sequence windows centered on the corresponding phosphosite. Human, mouse and rat phosphorylation sites were all considered to assess the biological and biochemical consequences of phosphorylation. The datasets were downloaded from the PhosphoSite Plus webpage on the 31^{st} of July, 2018 (phosphosite.org) (Hornbeck, P. V., et al. (2015). PhosphoSitePlus, 2014: mutations, PTMs and recalibrations. Nucleic acids research 43, D512-520, incorporated herein by reference in its entirety).

Identification of Protein Kinase Interactors

Protein kinase interactors were determined using the BioGRID database only considering protein-protein interactions for which two independent lines of evidence exist (Chatr-Aryamontri, A., et al. (2017). The BioGRID interaction database: 2017 update. Nucleic acids research 45, D369-D379, incorporated herein by reference in its entirety). To that end, the ‘BIOGRID-MV-Physical-3.5.165.tab2’ file was downloaded on Oct. 6, 2018 and mined for protein kinase interactions through matching against the gene name in the MaxQuant output files.

Determining Kinase Activation States

The activation state of a kinase was considered differentially regulated when either 1) regulatory sites on a kinase, 2) corresponding sites on a kinase or kinase interactor with a known kinase-substrate relationship, or 3) a known kinase interactor changed in expression between experiment conditions (T-test significant, see also ‘Functional phosphosites and kinase-substrate relationships’, ‘Identification of protein kinase interactors’, and ‘Quantitation and Statistical Analysis’.)

Kinome Dendrograms

Kinome dendrograms were prepared using the KinMap web application (kinhub.org/kinmap/) (Eid, S., Turk, S., Volkamer, A., Rippmann, F., and Fulle, S. (2017). KinMap: a web-based tool for interactive navigation through human kinome data. BMC Bioinformatics 18, 16, incorporated herein by reference in its entirety.)

Construction of Interaction Network Graphs

Protein-protein interaction network graphs were plotted with the STRING web application (v10.0, string-db.org/) (Szklarczyk, D., et al. (2015). STRING v10: protein-protein interaction networks, integrated over the tree of life. Nucleic acids research 43, D447-452). Solid edges shown in network figures represent the ‘confidence’ in the existence of a physical interaction

### Quantitation and Statistical Analysis

Differences between sample populations were quantified with a two-tailed two sample Student's T-test. For testing of proteomics data, or where indicated, BH correction for multiple hypothesis testing (FDR=0.05) was applied.

### Data and Software Availability

MS raw files and MaxQuant/Andromeda output files were deposited in the MassIVE repository under the dataset ID: MSV000083236. Drug response-kinase pathway interaction data can be viewed interactively via the Shiny web application (Lau, H.-T., et al. (2019). Kinome features, signaling pathways, and drug response in HCC (at quantbiology.org/hcckinome) (Ong Lab), incorporated herein by reference in its entirety); this resource allows users to view the association of signaling pathways, kinome features, and kinase inhibitors across the complete dataset.

### Example 2

As described in Example 1, the kinobead/LC-MS platform was implemented and identified differences in kinase expression, kinase phosphorylation, and kinase pathway activity between epithelial (i.e., drug sensitive) and mesenchymal (i.e., drug insensitive) HCC states. Among the differences were 196 kinases with differential expression, 72 of which are enriched in the drug-resistant mesenchymal cells. Example 1 specifically discusses a selected subset of the kinases found to be differentially expressed and/or activated in drug-insensitive mesenchymal cells, see, e.g., Table 2, including AXL, MET, EPHB2, FYN, AKT3, CAMK1D, NUAK1, NUAK2, EPHA4, CAMK1D, FYN, NEK3, CDK3, PLK1, CHEK1, EGFR, HIPK2, TNK2, LYN, PTK2, MAP3K12, MAPK9, MAPK8, and FER (see “A comprehensive map of the EMT state-associated kinome” and FIG. 7B). Additional differentially expressed and/or activated kinases identified in the disclosed study include AAK1, CDK10, STK17B, and STK32B. See also Golkowski, M., et al. (2020) Cell Systems. 11(2):196-207.e7, incorporated herein by reference in its entirety. Each of these kinases are potential targets for therapeutic intervention. Example 1 describes further investigations into specific the roles of AXL, NUAK1, and NUAK2 in the EMT and demonstrates their utility as viable drug targets, especially in combination with other kinase inhibitor therapies, to overcome drug sensitivities in cancers.

This Example describes studies of an additional kinase, AAK1, that was found to be highly activated in the EMT for its utility as a target for cancer therapy.

Results and Discussion

Among other findings, the kinome-centric study of hepatocellular carcinoma (HCC) described in Example 1 revealed that an AAK1 kinase signaling complex is aberrantly activated in mesenchymal-like and therapy-resistant HCC cell lines. To test if this kinase complex is causally involved in HCC cell EMT and therapy resistance and therefore may serve as a valuable novel drug target in HCC in other solid tumors, studies were conducted using RNA interference (RNAi) of AAK1 complex members, followed by molecular profiling and phenotypic screening.

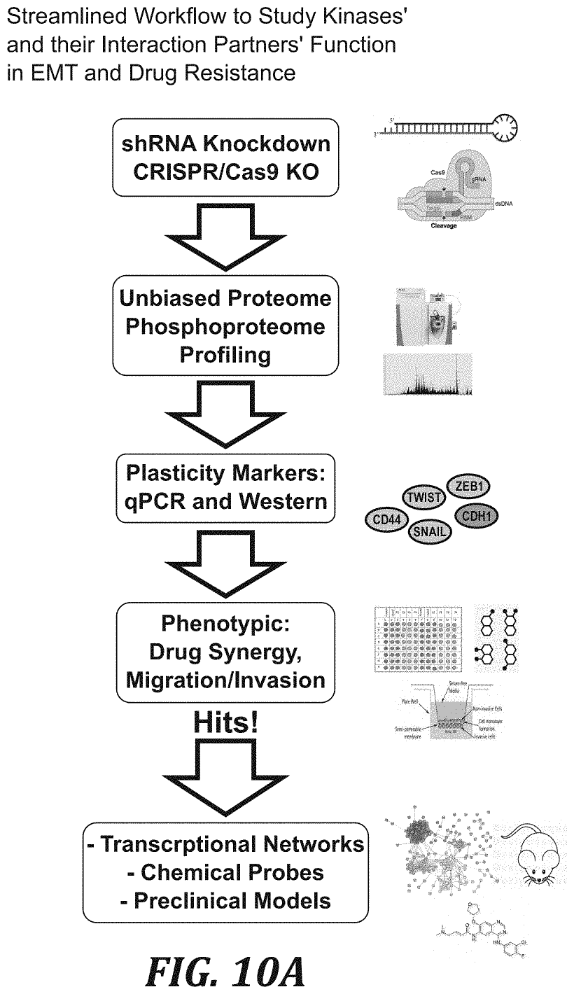

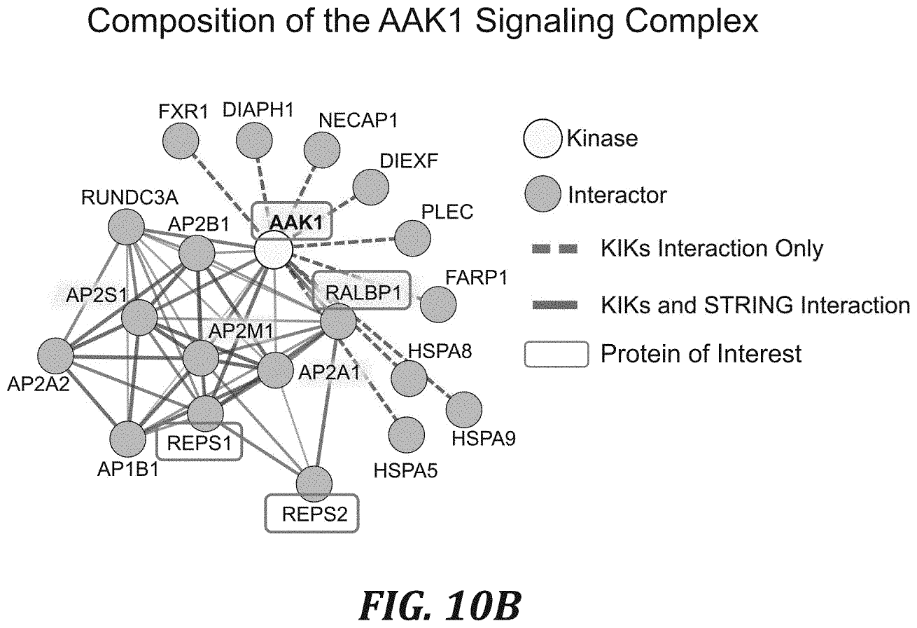

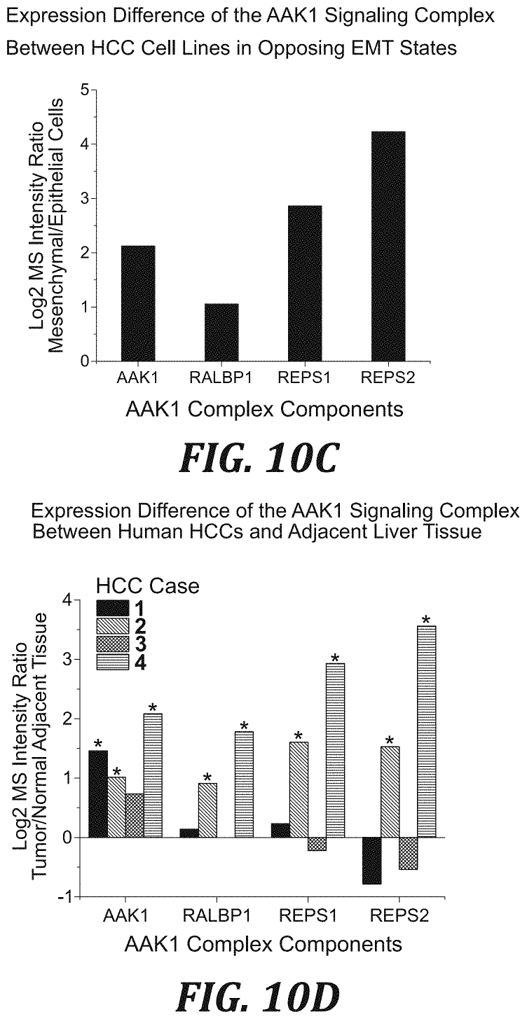

The approach to validate the role AAK1 is represented schematically in FIG. 10A. As illustrated, the general workflow pipeline is to test the biological function of kinases and their interaction partners function in EMT and cancer therapy resistance. (1) Kinases and their interaction partners are depleted from cell lines using either small hairpin RNAs (shRNAs) or CRISPR/Cas9 knockout. (2) Effects of RNAi knockdown (KD) or CRISPR knockout (KO) are quantified on cellular signaling pathways using unbiased MS-based kinome-centric and global proteome and phosphoproteome profiling. (3) KD/KO effects on EMT state are studied by quantifying 12 well-known EMT and stemness markers such as CDH1, VIM, SNAI2 and TWIST1 using qPCR and western blotting. (4) KD/KO effects are confirmed/determined on cancer therapy resistance by treating KD/KO cell lines with 15 clinical and pre-clinical HCC drugs, quantifying cell viability using the Promega Cell Titer Glo 2.0 assay. This assay aims to discover drug synergies that may be exploited to develop novel combination therapies. (5) KD/KO effects are measured on cell migration and invasion by conducting trans-well migration and wound scratch assays. These assays can discover novel applications for anti-metastatic drugs. Kinases and their interaction partners that show an effect in the named assays are promising novel drug targets to develop efficient combination therapies to kill cancer cells or anti-metastatic drugs. Future experiments can further confirm the roles and targeting value of these kinases by studying in greater detail using mRNA sequencing and chemical genetic manipulations in vitro and in pre-clinical cancer models to learn more about their biology and translational potential. This general workflow is target-agnostic can be used to study any of the kinases or kinase interactors disclosed Example 1.

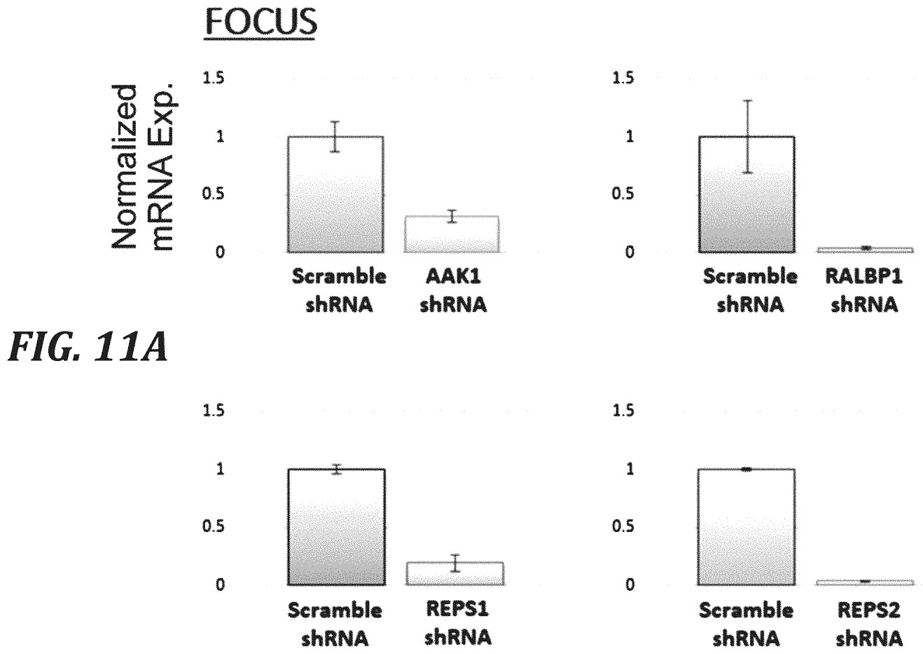

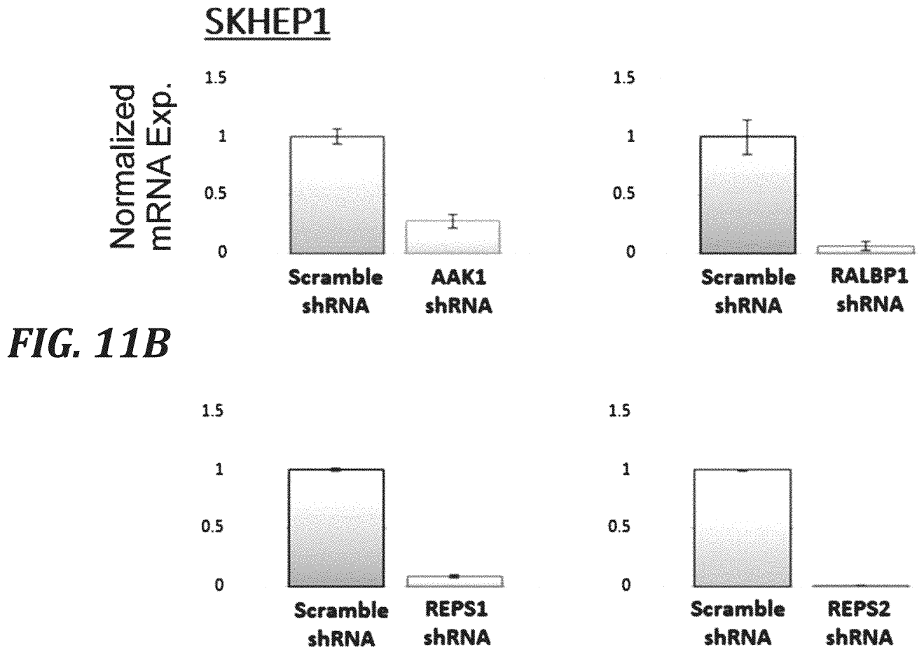

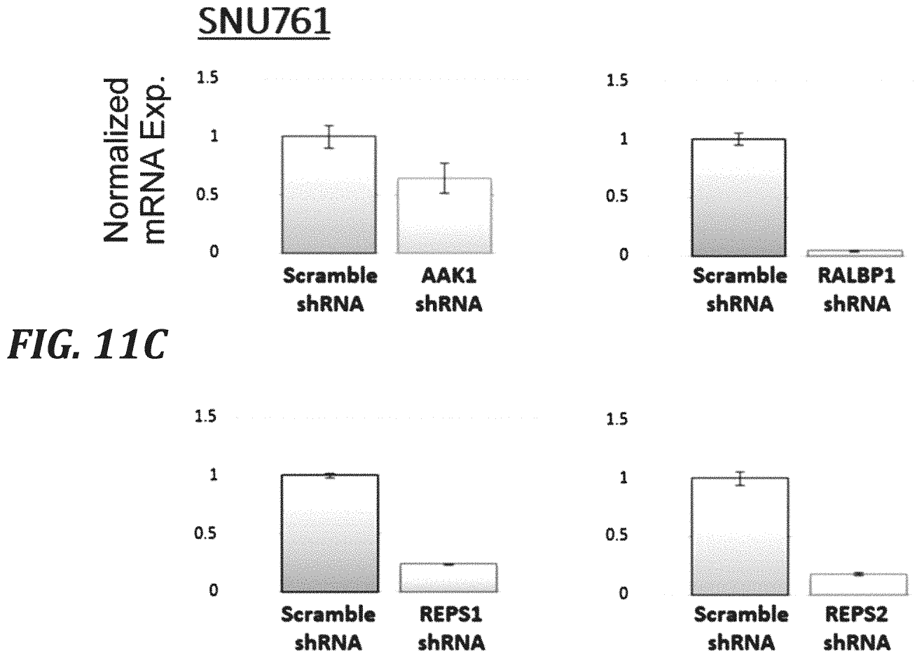

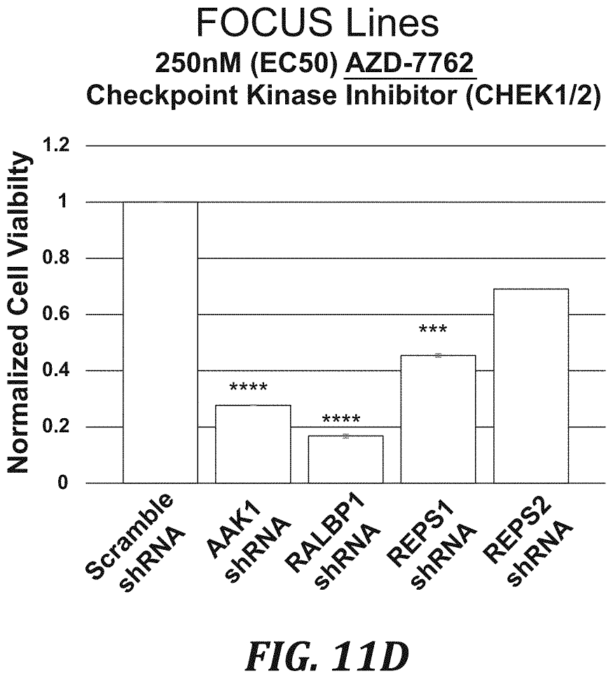

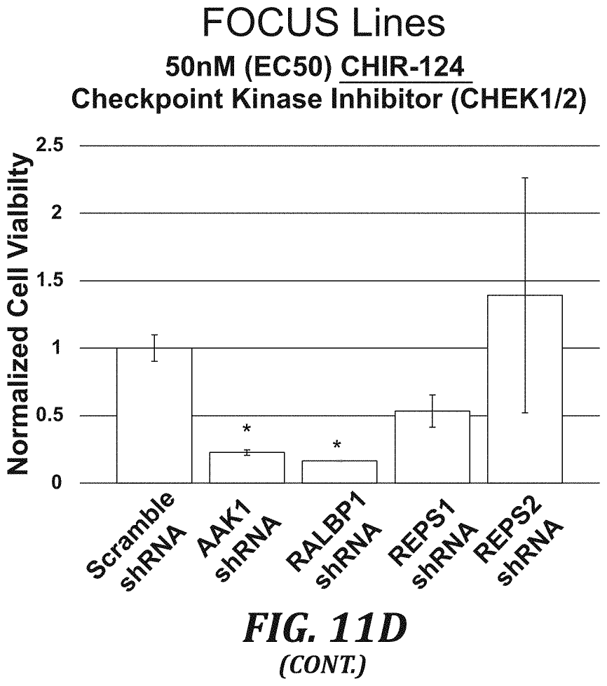

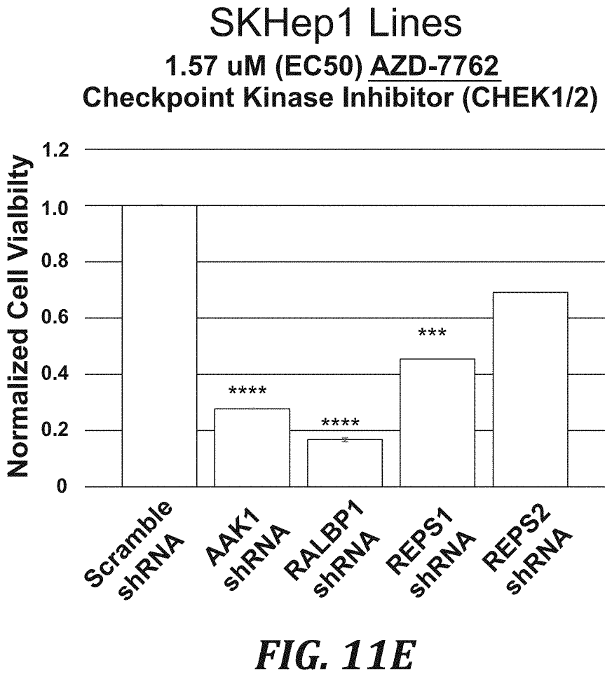

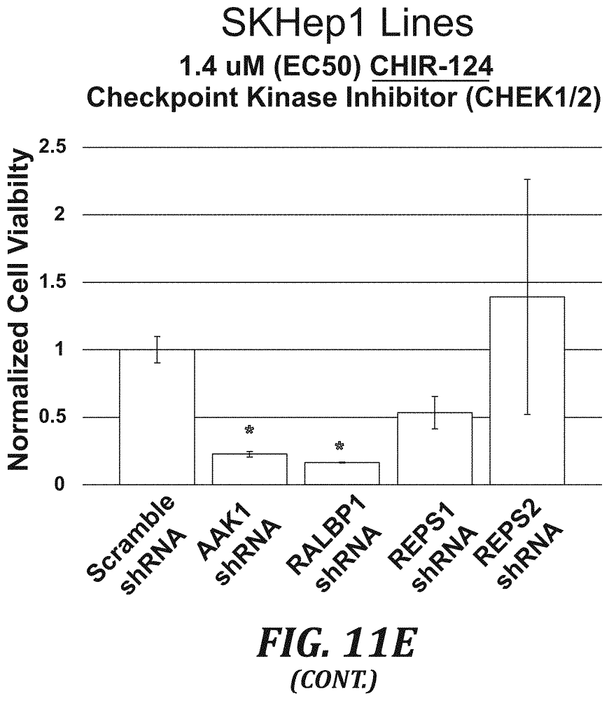

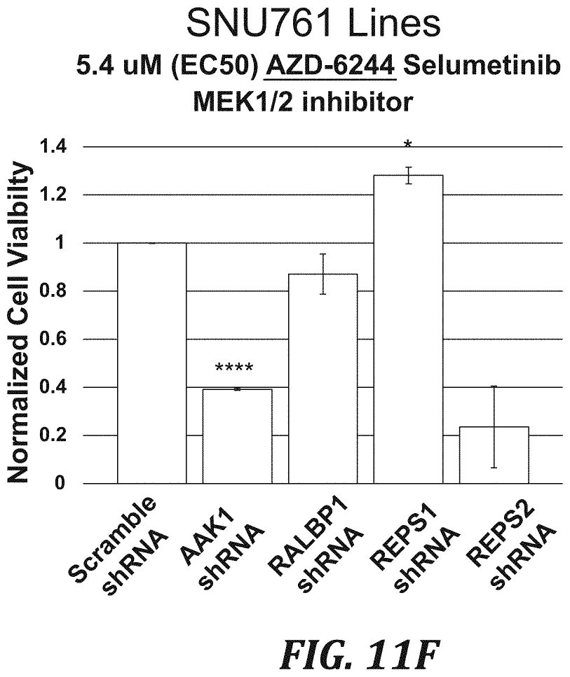

FIG. 10B illustrates the relationships between the compositions of the AAK1 signaling complex in HCC cells, as determined by kinobead/LC-MS kinome activity profiling described in Example 1. Proteins known to be part of oncogenic signaling pathways that may promote EMT are highlighted. These proteins were selected for detailed analysis. FIG. 11C graphically illustrates the difference in protein expression of AAK1 and its interaction partners RALBP1, REPS 1, and REPS2 between the aggregate of 7 drug-sensitive epithelial-like and the aggregate of 10 drug-resistant mesenchymal-like HCC cell lines, as determined by kinobead/LC-MS kinome activity profiling (see Example 1). The data suggests that the AAK1 complex is specifically expressed in mesenchymal like cells, indicating that AAK1 signaling is active in these cells. Similarly, FIG. 11D graphically illustrates the difference in protein expression of AAK1 and its interaction partners comparing four human HCC tumor tissue samples with paired normal adjacent liver tissue using the kinome kinobead/LC-MS kinome profiling (see Example 1). The results demonstrate that the AAK1 signaling complex is active in at least 2/4 human tumors, strongly supporting our hypothesis that AAK1 may play a causal role in the progression of HCC patients' tumors.

Next, the impact of interruption of AAK1 functionality on the EMT was explored. FIGS. 11A-11C graphically illustrate results from qPCR analysis of three different mesenchymal HCC cell lines (FOCUS (11A), SKHep1 (11B), SNU761 (11C)) that have been stably transfected with a plasmid encoding shRNAs that specifically target AAK1 and its interaction partners or a scrambled shRNA (control). These results demonstrate that a 2- to 10-fold knockdown of each target mRNA is achieved, making these cell lines suitable models to test the function of AAK1 and its interaction partners in our downstream analysis. The next step was to assess the ability of an AAK1 knockdown to sensitize the cells to extant KI targeting therapeutics. FIGS. 11D-11F graphically illustrate drug synergy of knockdown of AAK1 and its interaction partners RALPB1, REPS1 and REPS2 sensitizes therapy-resistant and mesenchymal-like HCC cells to treatment with targeted cancer drugs in three different HCC cell lines (FOCUS (11D), SKHep1 (11E), SNU761 (11F)). Briefly, RNAi lines and scramble control were treated with checkpoint kinase inhibitors (CHEK1/2) AZD7762 and CHIR-124 or the MEK1/2 inhibitor AZD6244 (Selumetinib) for 72 hours and cell viability quantified using Promega's cell titer Glo 2.0 assay (see also Example 1).

Conclusions and Outlook

The disclosed MS-based proteome profiling, EMT marker quantification and drug synergy testing suggest that AAK1 and its interaction partners promote the EMT and control HCC cell resistance to specific targeted drugs, particularly cell cycle checkpoint kinase (CHEK1/2) inhibitors. CHEK1 and 2 control cell survival in cancer cells that rapidly divide, and experience replication stress, as is typically observed in epithelial-like cancer cells. The disclose data support the hypothesis that the AAK1 signaling complex promotes the EMT and that inhibition of the complex reverts cells to the epithelial, therapy sensitive state. Collectively, these data demonstrate that the AAK1 signaling complex is an attractive novel drug target candidate for combination therapies in mesenchymal-like HCCs. Going forward, cell migration/invasion assays and global mRNA sequencing of KD lines can be used to validate the causal involvement of the AAK1 signaling complex in EMT. This will be followed by medicinal chemistry to identify selective inhibitors of the AAK1 complex. Such inhibitors can then be used to test combination therapies in preclinical models such as PDX mice and tumor slice cultures. Success in pre-clinical models would suggest high translational value of combination strategies targeting the AAK1 complex and could warrant early-stage clinical trials.

### Example 3

As indicated above, Example 1 describes a study revealing kinases that can influence the drug-insensitivity in cancers, e.g., HCC. This Example describes an investigation of an additional kinase, CAMK1D, that was found to be upregulated and active in the EMT for its utility as a target for cancer therapy.

Results and Discussion

The kinome-centric study of hepatocellular carcinoma (HCC) described in Example 1 revealed that the kinase CAMK1D was associated with EMT and was significantly upregulated in mesenchymal HCC cells versus epithelial HCC cells. Thus, CAMK1D was studied in more detail following the general workflow illustrated in FIG. 10A.

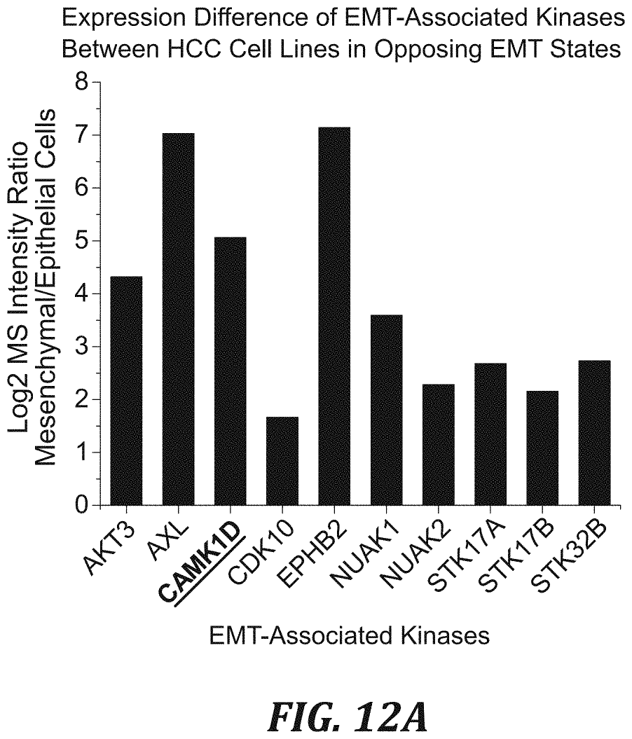

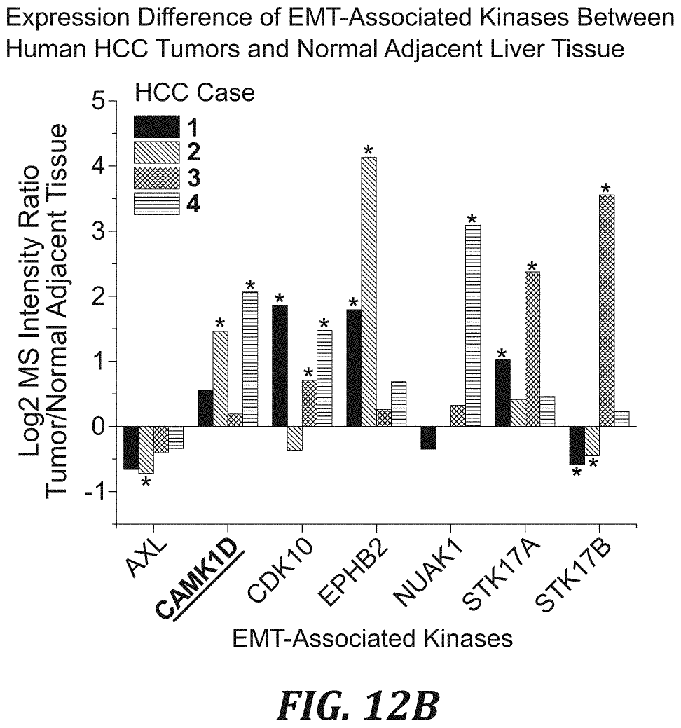

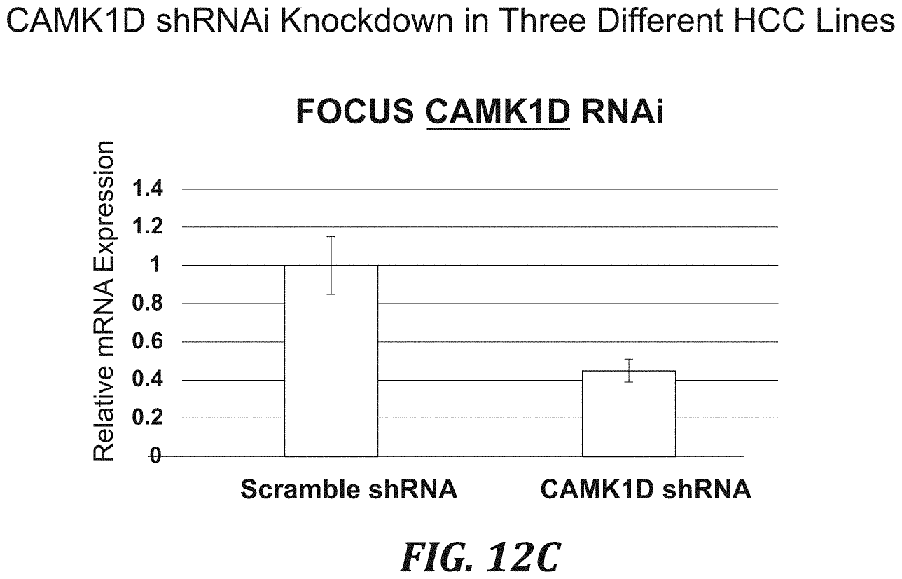

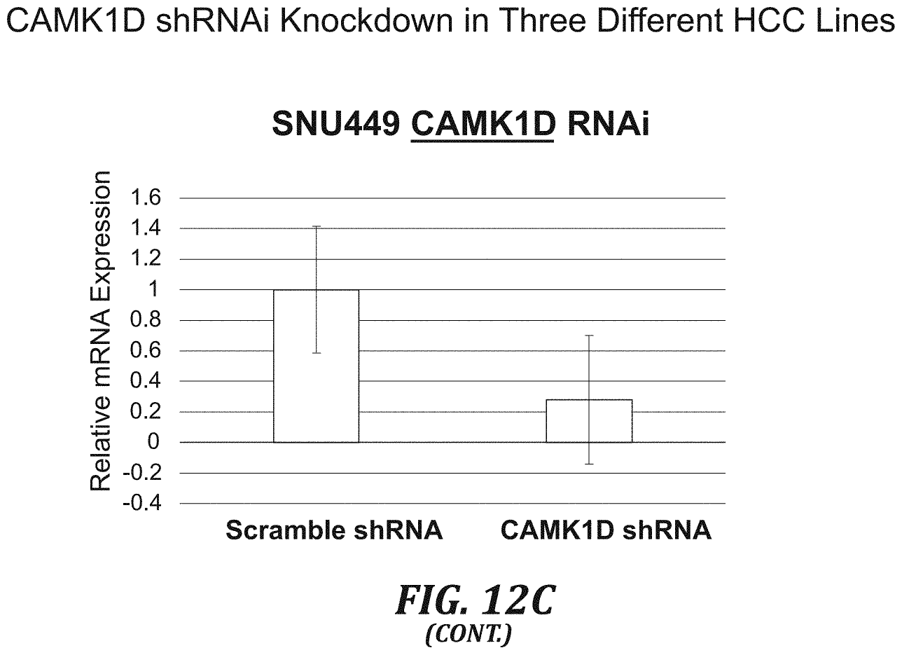

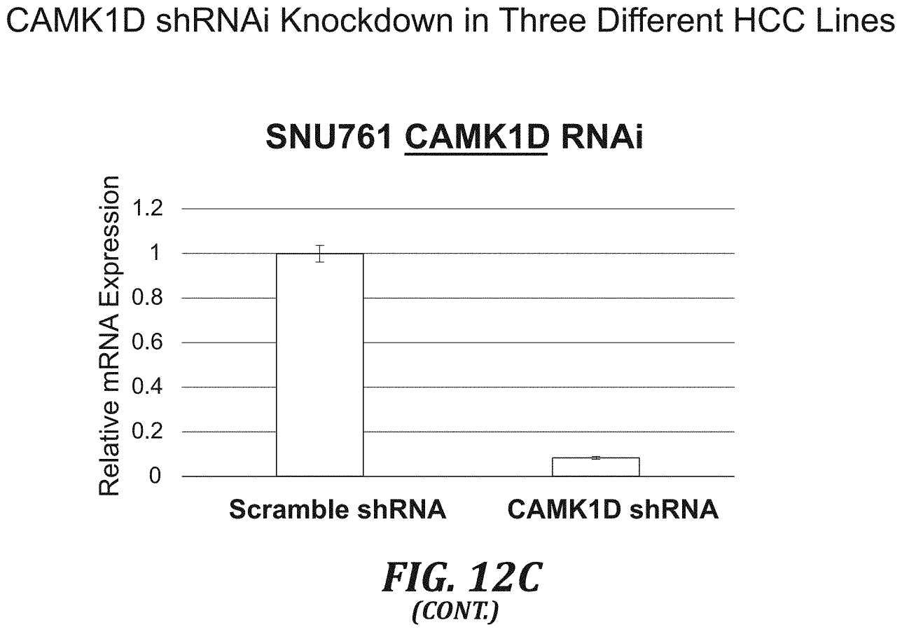

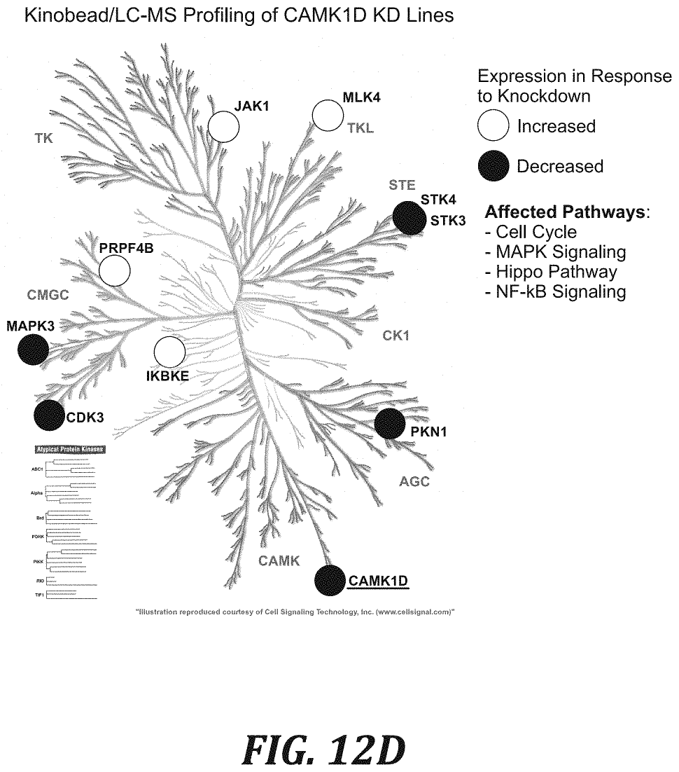

FIG. 12A graphically illustrates the difference in protein expression of EMT-associated kinases AKT3, AXL, CAMK1D, CDK10, EPHB2, NUAK1, NUAK2, STK17A, STK17B, and STK32B comparing 7 drug-sensitive epithelial-like and 7 drug-resistant mesenchymal-like HCC cell lines using the kinome kinobead/LC-MS kinome profiling technology. The data show that the serine/threonine kinase CAMK1D is among the most highly overexpressed in mesenchymal HCC cells. Next, expression of select kinases including CAMK1D was quantified in and compared between human HCC tumors and normal adjacent liver tissue. Specifically, FIG. 12B graphically illustrates differences in protein expression of EMT-associated kinases, including CAMK1D, comparing four human HCC tumor tissue samples with paired normal adjacent liver tissue using the kinome kinobead/LC-MS kinome profiling technology (see Example 1).

FIG. 12C is a series of graphs showing results from qPCR analysis of mesenchymal FOCUS, SNU449 and SNU761 cell lines that have been stable transfected with a plasmid encoding shRNAs that specifically target CAMK1D (see FIG. 10A) or a scramble shRNA (control). The results demonstrate that a 2- to 10-fold knockdown of CAMK1D was achieved, making these cell lines suitable models to test the function of this kinase in our downstream assays. FIG. 12D illustrates results of kinobead/LC-MS kinome activity profiling of FOCUS, SNU449 and SNU761 CAMK1D KD cell lines. Kinases that change in expression in response CAMK1D knockdown in at least 2 of 3 tested cell lines are overlayed with the human kinome dendrogram. Pathways these kinases regulate are highlighted.

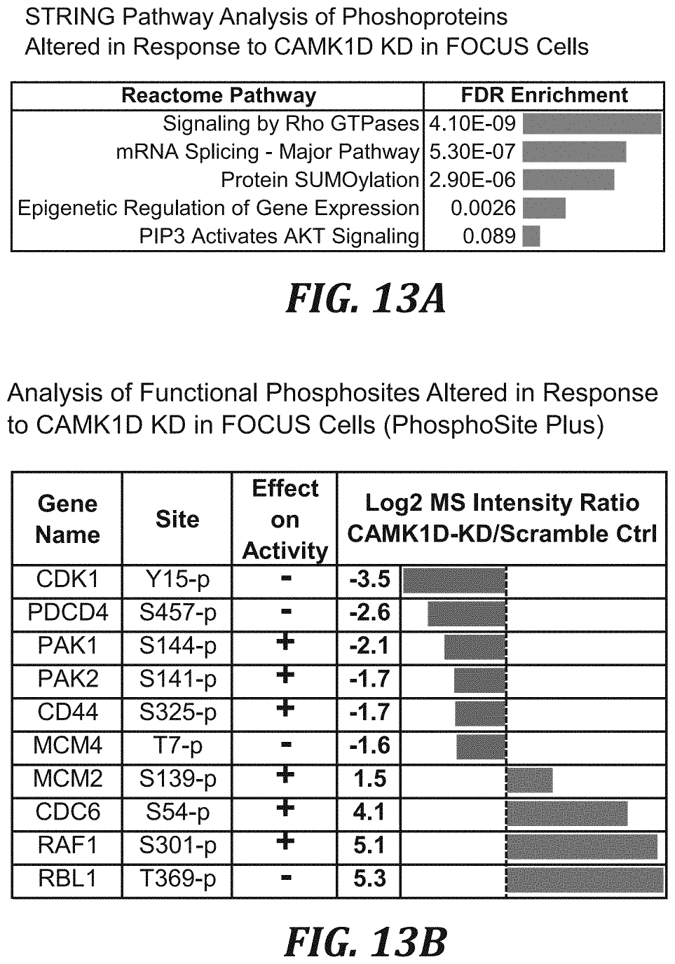

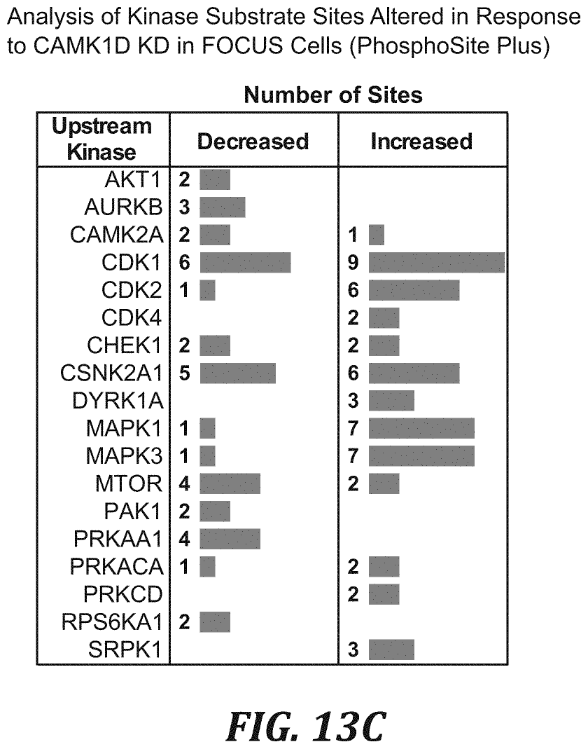

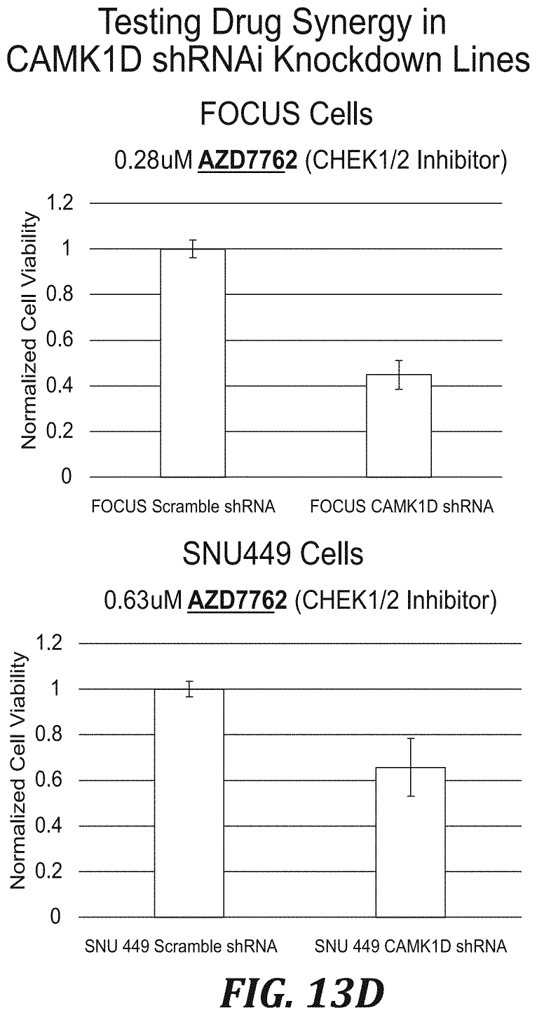

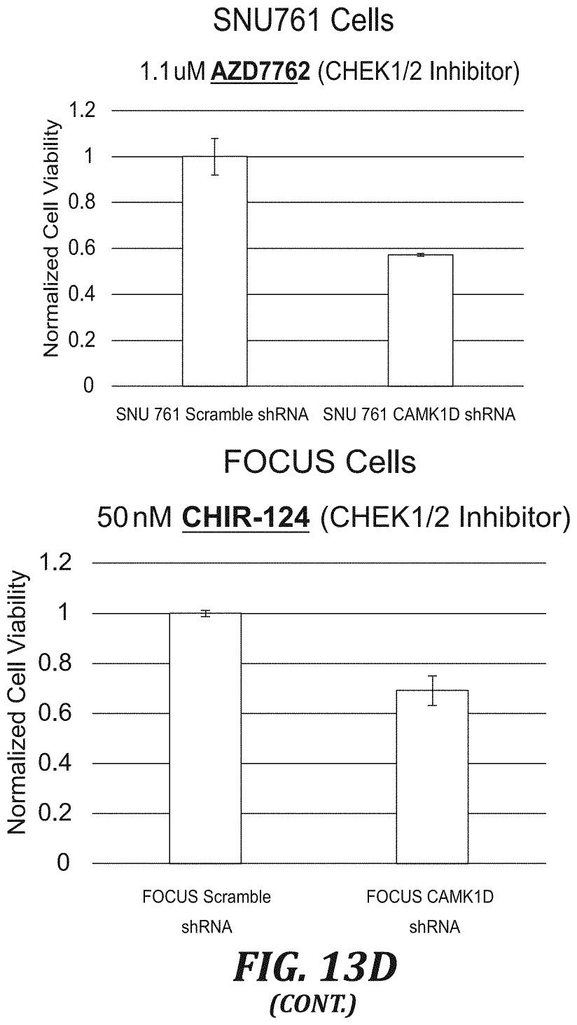

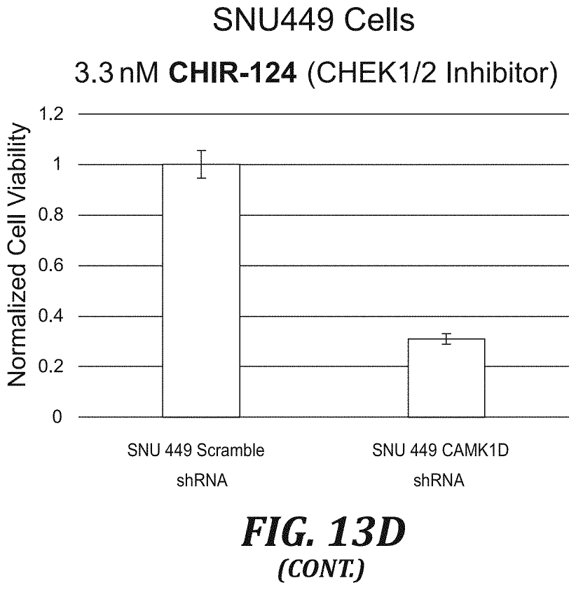

Further analyses of CAMK1D were conducted to further investigate its role in EMT and potential therapeutic target. FIG. 13A illustrates results of a STRING pathway analysis using Reactome pathways of proteins differentially phosphorylated between FOCUS scramble shRNA control cells and FOCUS CAMK1D KD cells. Phosphoproteins were quantified using global MS-based phosphoproteomics according to our previously published protocol. See Golkowski, M. et al. Kinobead/LC-MS Phosphokinome Profiling Enables Rapid Analyses of Kinase-Dependent Cell Signaling Networks. Journal of proteome research 19, 1235-1247 (2020), incorporated herein by reference in its entirety. The results indicate that CAMK1D knockdown affect 1,734 phosphorylation sites on 659 proteins that control pathways such as Rho-GTPases (cell migration), mRNA splicing, protein SUMOylation and epigenetic regulation of gene expression, as well as PI3K-AKT signaling (cell survival). FIG. 13B illustrates a database alignment of CAMK1D-dependent phosphopeptide expression (see FIG. 10A) using the PhosphositesPlus functional phosphorylation site dataset. The results show that CAMK1D activity affects proteins that control the cell cycle (CDK1, MCM2/4, CDCl₆, RAF1), cell migration (PAK1/2, CD44) and cell survival (PDCD4, RBL1). FIG. 13C illustrates a database alignment of CAMK1D-dependent phosphopeptide expression (see FIG. 10A) using the PhosphositesPlus kinase-substrate relationship dataset. The results show that CAMK1D activity affects the activity of other kinases. Most prominently CAMK1D negatively affect the activity of CDK1/2 and MAK1/3 (cell cycle and mitogenic signaling) and positively affect the activity of MTOR, AKT and PAK1 (metabolism, survival, and cell migration). Finally, FIG. 13D is a series of graphs illustrating result of testing whether shRNAi knockdown of CAMK1D sensitizes mesenchymal HCC cells to treatment with targeted cancer drugs. Briefly, RNAi lines and scramble control were treated with checkpoint kinase inhibitors (CHEK1/2) AZD7762 or CHIR-124 for 72 hours and cell viability quantified using Promega's cell titer Glo 2.0 assay. The results show that CAMK1D knockdown sensitizes cell to both AZD7762 and CHIR-124.

Conclusions and Outlook

MS-based proteome profiling drug synergy testing suggest that CAMK1D positively regulates cell migration and survival, negatively regulates the cell cycle, and increases HCC cell resistance to specific targeted drugs, particularly cell cycle checkpoint kinase (CHEK1/2) inhibitors. CHEK1 and 2 control cell survival in cancer cells that rapidly divide, and experience replication stress, as is typically observed in epithelial-like cancer cells. It was concluded that CAMK1D inhibition reduces cell survival signaling, thus boosting the efficacy targeted drugs. Collectively, this demonstrates that CAMK1D is an attractive novel drug target candidate for combination therapies in mesenchymal-like HCCs. Future investigations to further characterize the anti-cancer therapeutic effects include cell migration/invasion assays and global mRNA sequencing of CAMK1D KD lines to validate the causal involvement of CAMK1D in survival signaling and therapy resistance. Medicinal chemistry efforts have led to development of a selective inhibitor of CAMK1D (see Fromont, C. et al. Discovery of Highly Selective Inhibitors of Calmodulin-Dependent Kinases That Restore Insulin Sensitivity in the Diet-Induced Obesity in Vivo Mouse Model. J Med Chem 63, 6784-6801, (2020), incorporated herein by reference in its entirety). Future assays will test various combination therapies in preclinical models such as PDX mice and tumor slice cultures. Success in pre-clinical models would suggest high translational value of combination strategies targeting CAMK1D and could warrant early-stage clinical trials.

### Example 4

As indicated above, Example 1 describes a study revealing kinases that can influence the drug-insensitivity in cancers, e.g., HCC. This Example describes an investigation of additional kinases, CDK10, STK17A, and STK32B, that were found to be upregulated and active in the EMT for its utility as a target for cancer therapy.

Results and Discussion

The kinome-centric study of hepatocellular carcinoma (HCC) described in Example 1 revealed that the kinase CDK10, STK17A, and STK32B were independently associated with EMT and were significantly upregulated in mesenchymal HCC cells versus epithelial HCC cells. Thus, preliminary studies of CDK10, STK17A, and STK32B were conducted following the general workflow illustrated in FIG. 10A.

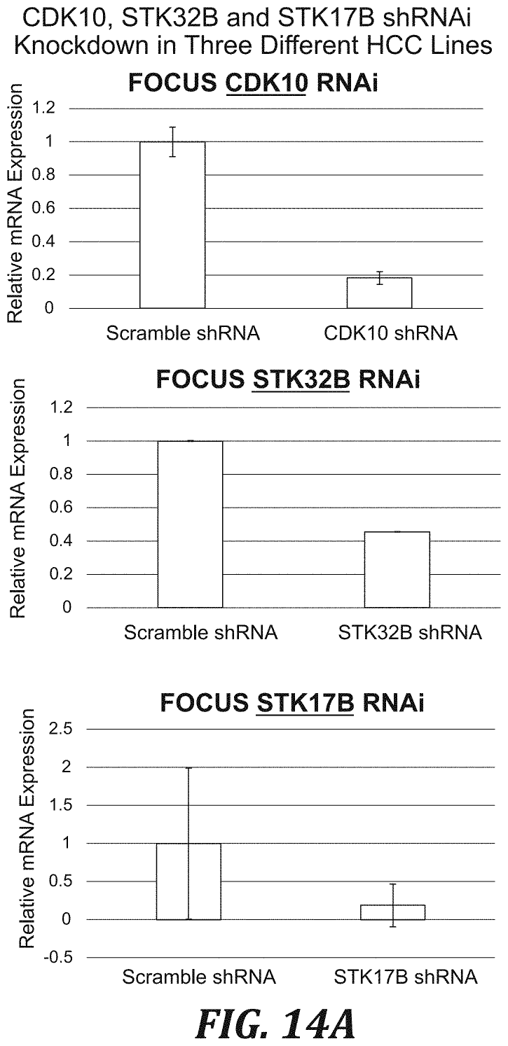

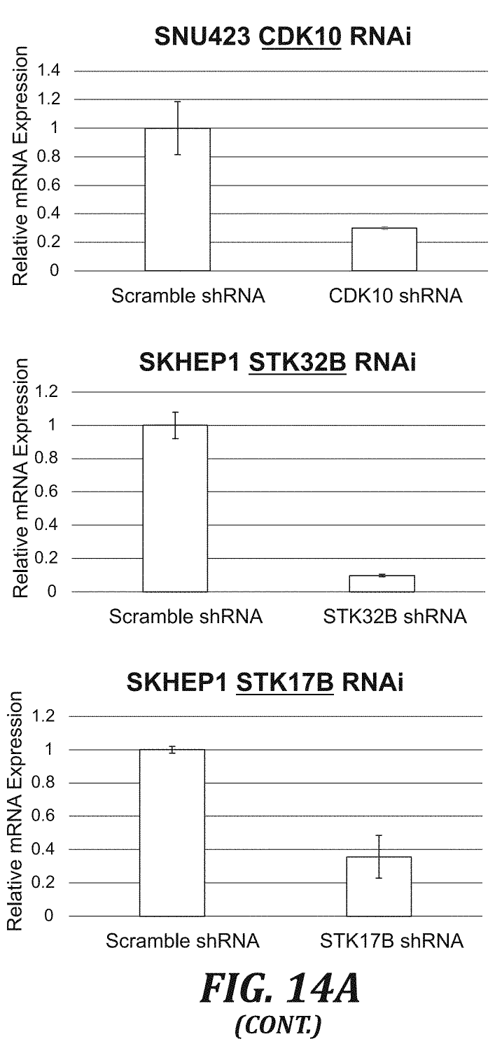

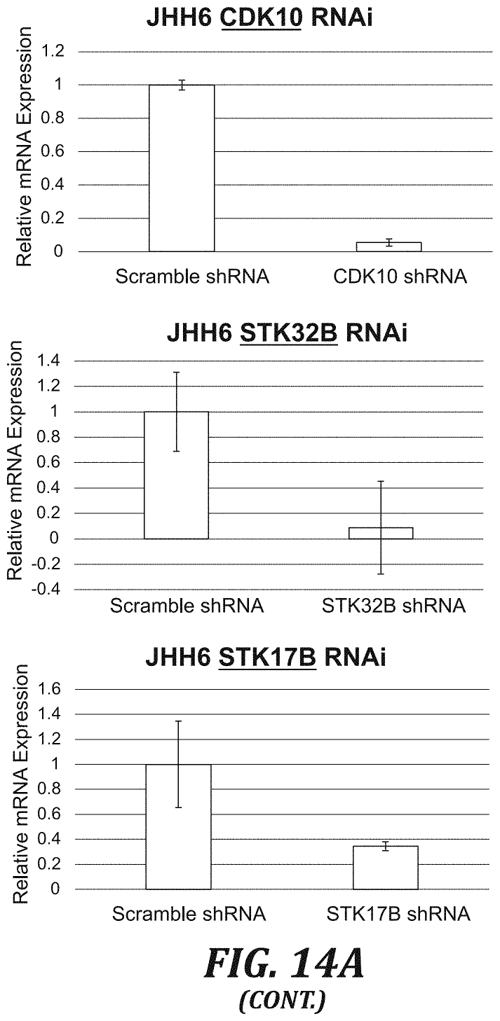

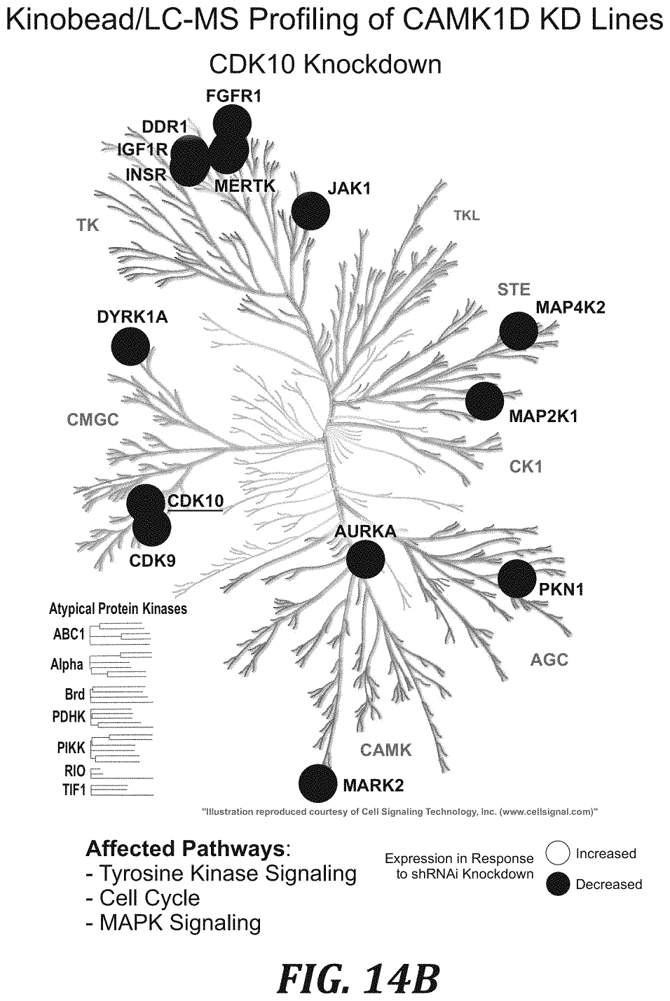

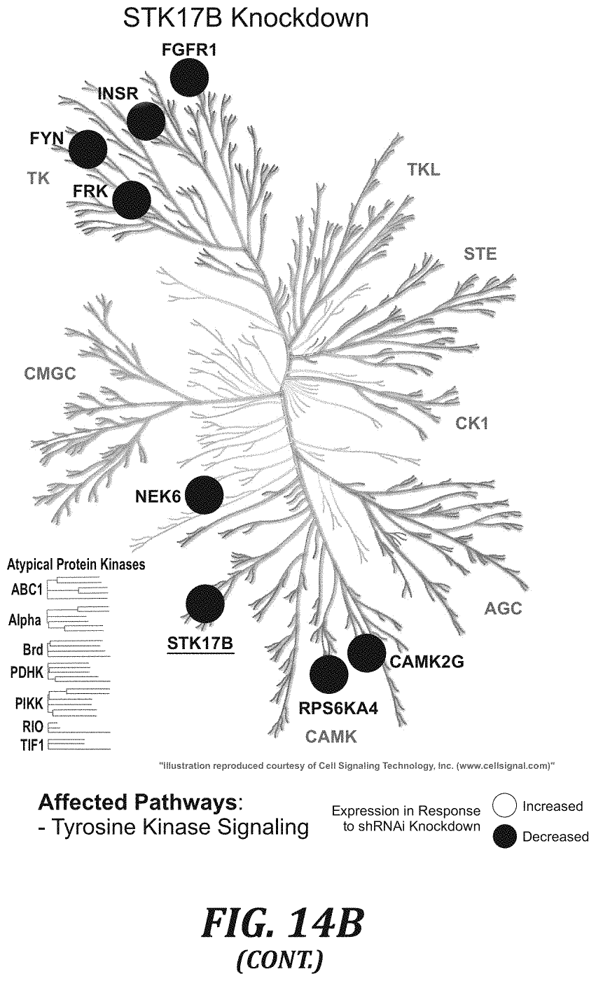

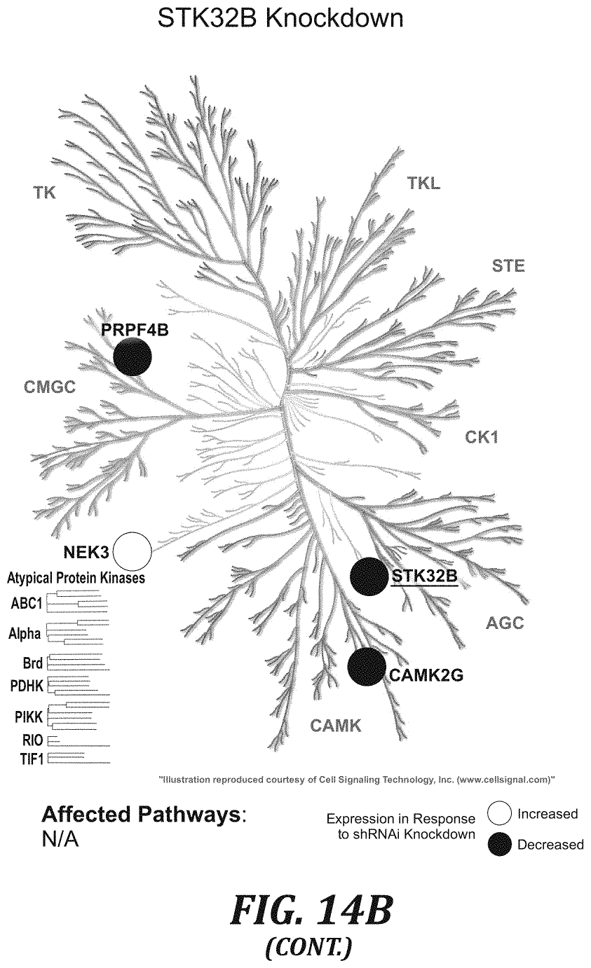

FIG. 14A provides a series of graphs showing results from qPCR analysis of mesenchymal FOCUS, SNU423, JHH6, and SKHep1 cell lines that were stably transfected with a plasmid encoding shRNAs that specifically target CDK10, STK17B, or STK32B (see FIG. 10A) or a scrambled shRNA (as control). The results demonstrate that a 2- to 10-fold knockdown of target kinases was achieved, establishing these cell lines as suitable models to test the function of this kinase in downstream assays. FIG. 14B illustrates the results of kinobead/LC-MS kinome activity profiling of FOCUS, SNU423, JHH6 and SKHep1 cell lines in which CDK10, STK17B or STK32B have been separately depleted by shRNAi. Kinases that change in expression in response kinase knockdown in at least 2 of 3 tested cell lines are overlayed with the human kinome dendrogram. Pathways these kinases regulate are highlighted.

Conclusions and Outlook

MS-based proteomics profiling provides preliminary data demonstrating that CDK10 and STK17B regulate major oncogenic pathways and suggest that targeting these kinases with inhibitors could be therapeutically beneficial. In contrast, STK32B knockdown did not yield a conclusive result and requires more characterization. In future experiments, all three kinases will be subjected to global proteome and phosphoproteome profiling and phenotypic screening according to the general workflow to test their translational potential (see FIG. 10A). Results presented in this and the other examples demonstrate that the kinobead/LC-MS kinome analysis successfully and consistently revealed functional associations between kinase expression and/or activity and the transition from epithelial phenotype to mesenchymal phenotype in cancer cells (e.g., HCC cells), thus demonstrating their potential as therapeutic targeting to reverse the insensitivity to kinase-based therapeutics observed in the mesenchymal phenotypes. This analysis suggests the potential utility of the kinases listed in, e.g., Table 2, as potential therapeutic targets to enhance efficacy of new and extant kinase-based therapeutics.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of reducing resistance in a cancer cell to a chemotherapeutic agent, comprising contacting the cell with an agent that inhibits the expression or function of an epithelial-mesenchymal transition (EMT)-associated kinase.

2. The method of claim 1, wherein the chemotherapeutic agent is kinase inhibitor.

3. The method of claim 2, wherein the kinase inhibitor is selected from Table 1.

4. The method of claim 2, wherein the kinase inhibitor is an inhibitor of a kinase selected from EGFR, SRC, c-MET, RAF, IGH1R, MEK1/2, PI3K, CHECK1/2, PLK1, CDK1/2, FGFR, mTOR, and AURK.

5. The method of claim 2, wherein the kinase inhibitor is selected from sorafenib, regorafenib, lenvatinib, cabozantinib, dinaciclib, tezolizumab, ramucirumab, and becacizumab.

6. The method of claim 1, wherein the cancer cell is a hepatocellular carcinoma cell.

7. The method of claim 1, wherein contacting the cell with the agent prevents or reverses transition of the cancer cell from an epithelial phenotype to a mesenchymal phenotype.

8. The method of claim 1, wherein the EMT-associated kinase is selected from the kinases listed in Table 2.

9. The method of claim 1, wherein the EMT-associated kinase is selected from AXL, MET, EPHB2, FYN, AKT3, CAMK1D, NUAK1, NUAK2, EPHA4, CAMK1D, FYN, NEK3, CDK3, PLK1, CHEK1, EGFR, HIPK2, TNK2, LYN, PTK2, MAP3K12, MAPK9, MAPK8, FER, AAK1, CDK10, STK17B, and STK32B.

10. The method of claim 1, further comprising contacting the cell with the chemotherapeutic agent.

11. The method of claim 1, wherein the cell is contacted in vivo in a subject with cancer, and the method comprises administering a therapeutically effective amount of the agent that inhibits the expression or function of the EMT-associated kinase.

12. A method of enhancing sensitivity of a cancer cell to a kinase inhibitor therapy in a subject in need thereof, comprising administering to the subject an effective amount of an agent that inhibits the expression or function of an epithelial-mesenchymal transition (EMT)-associated kinase.

13. The method of claim 12, wherein the kinase inhibitor is selected from Table 1.

14. The method of claim 12, wherein the kinase inhibitor is an inhibitor of a kinase selected from EGFR, SRC, c-MET, RAF, IGH1R, MEK1/2, PI3K, CHECK1/2, PLK1, CDK1/2, FGFR, mTOR, and AURK.

15. The method of claim 12, wherein the kinase inhibitor is selected from sorafenib, regorafenib, lenvatinib, cabozantinib, dinaciclib, tezolizumab, ramucirumab, and becacizumab.

16. The method of claim 12, wherein the cancer cell is a hepatocellular carcinoma cell.

17. The method of claim 12, wherein administering the agent prevents or reverses transition of the cancer cell from an epithelial phenotype to a mesenchymal transition phenotype.

18. The method of claim 12, wherein the EMT-associated kinase is selected from the kinases listed in Table 2.

19. The method of claim 12, wherein the EMT-associated kinase is selected from AXL, MET, EPHB2, FYN, AKT3, CAMK1D, NUAK1, NUAK2, EPHA4, CAMK1D, FYN, NEK3, CDK3, PLK1, CHEK1, EGFR, HIPK2, TNK2, LYN, PTK2, MAP3K12, MAPK9, MAPK8, FER, AAK1, CDK10, STK17B, and STK32B.

20. The method of claim 12, wherein the method is a method for treating the cancer and further comprises administering a therapeutically effective amount of the chemotherapeutic agent to the subject.
